(19) United States
(12) Patent Application Publication
(10) Pub. No.: US 2025/0011805 A1
(43) Pub. Date: Jan. 9, 2025

(54) PATHOGEN RESISTANCE IN PLANTS

(71) Applicant: Fortiphyte, Inc., Berkeley, CA (US)

(72) Inventors: Alexander Christiaan SCHULTINK, Berkeley, CA (US); Laura A. WETZEL, Berkeley, CA (US)

(21) Appl. No.: 18/496,072

(22) Filed: Oct. 27, 2023

Related U.S. Application Data
(63) Continuation of application No. 18/062,538, filed on Dec. 6, 2022, now abandoned.
Application No. 18/062,538 is a continuation of application No. 17/512,329, filed on Oct. 27, 2021, now Pat. No. 11,530,419.
(60) Provisional application No. 63/108,023, filed on Oct. 30, 2020.

(51) Int. Cl. C12N 15/82; C12N 15/11; C12N 15/90

(52) U.S. Cl. CPC C12N 15/8282; C12N 15/111; C12N 15/902; C12N 2310/20

(57) ABSTRACT

The present disclosure provides an isolated, recombinant, or synthetic polynucleotide comprising a FIT1 protein, and homologs, fragments, and variations thereof. The disclosure further relates to transgenic plants, plant parts, and plant cells comprising one or more of these polynucleotides, and exhibit resistance or tolerance to a pathogen, such as Phakopsora pachyrhizi. The disclosure further relates to methods of genetically engineering a pathogen resistance or tolerance trait in a plant, plant part, or plant cell, comprising targeted gene editing of a FIT1 homolog, and plants produced therefrom. The disclosure further relates to methods for identifying new functional FIT1 genes and/or alleles thereof.

### CROSS-REFERENCE TO RELATED APPLICATIONS

This application is a continuation of U.S. application Ser. No. 18/062,538 filed on Dec. 6, 2022, which is a continuation of U.S. application Ser. No. 17/512,329 filed on Oct. 27, 2021 (U.S. Pat. No. 11,530,419; issue date Dec. 20, 2022), which claims priority to and benefit of U.S. Provisional Application No. 63/108,023, filed on Oct. 30, 2020, which are hereby incorporated by reference in their entirety for all purposes.

### STATEMENT REGARDING FEDERALLY SPONSORED RESEARCH OR DEVELOPMENT

This invention was made with Government support under 1844088 by the National Science Foundation. The Government has certain rights to this invention.

### REFERENCE TO AN ELECTRONIC SEQUENCE LISTING

The contents of the electronic sequence listing (FOTI_001_03US_SeqList_ST26.xml; Size: 150,881 bytes; and Date of Creation: Oct. 26, 2023) are herein incorporated by reference in its entirety.

### TECHNICAL FIELD

The disclosure relates to the identification and use of nucleic acid sequences for pathogen resistance in plants.

### BACKGROUND

Pathogen effector proteins often convergently evolve to target the same or similar plant host proteins to promote virulence. Such proteins may be present in a diverse range of plant pathogens including but not limited to fungi, oomycetes, bacteria, nematodes or viruses.

An example of one such pathogen that causes harm to plants by secreting an effector protein is the obligate biotrophic fungus Phakopsora pachyrhizi (and to a lesser extent, the closely related fungus Phakopsora meibomiae), which causes Asian soybean rust (ASR). While soybeans make up the primary commercial crop affected by ASR, Phakopsora infects leaf tissue from a broad range of leguminous plants, including at least 17 genera (Slaminko et al., 2008). In general, rust fungi (order Pucciniales) constitute one of the most economically important groups of plant pathogens because of their larger range of host and genetic diversity. There are more than 6000 species of rust fungi that cause harm to many plant species, such as wheat (Puccinia spp.), common bean (Uromyces appendiculatus), soybean (Phakopsora pachyrhizi), and coffee (Hemileia vastatrix) (Aime et al., 2006).

Infection in commercial crops requires application of various fungicides, which are costly and not always effective. In Brazil alone, control of ASR costs $2 billion annually. Thus, there is a need for plant varieties that are resistant to fungal pathogens.

The foregoing examples of the related art and limitations related therewith are intended to be illustrative and not exclusive. Other limitations of the related art will become apparent to those of skill in the art upon a reading of the specification.

### SUMMARY

The present disclosure provides for an isolated, recombinant, or synthetic polynucleotide comprising a nucleic acid sequence encoding a functional FIT1 protein homologous to SEQ ID NO: 2. In some embodiments, the polynucleotide encodes a protein having at least 70% identity to SEQ ID NO: 2. In some aspects, the protein is selected from the group consisting of: SEQ ID NOs: 2, 4, 6, 8, 10, 12, 14, 16, 18, 20 and functional homologs thereof. In some aspects, the isolated, recombinant, or synthetic polynucleotide comprises a nucleic acid sequence selected from the group consisting of SEQ ID NOs: 1, 3, 5, 7, 9, 11, 13, 15, 17, 19 complements thereof, fragments thereof, and sequences at least 70% identical thereto. The disclosure further relates to genetic constructs comprising one or more of these sequences, and transgenic plants, plant parts, or plant cells comprising one or more of these sequences, wherein the plant, plant part, or plant cell is resistant or tolerant to a pathogen.

In another embodiment, the disclosure teaches a method of producing a plant, plant part, or plant cell having resistance or tolerance to a pathogen, wherein the method comprises transforming a plant, plant part, or plant cell with a polynucleotide encoding a functional FIT1 protein. In some aspects, the nucleotide sequence encoding the FIT1 protein has been codon optimized. In some aspects, the FIT1 protein comprises a selected from the group consisting of: SEQ ID NOs: 2, 4, 6, 8, 10, 12, 14, 16, 18, 20 and functional homologs thereof, or is encoded by an isolated, recombinant, or synthetic polynucleotide selected from the group consisting of SEQ ID NOs: 1, 3, 5, 7, 9, 11, 13, 15, 17, 19, complements thereof, fragments thereof, and sequences at least 70% identical thereto.

In another embodiment, the disclosure teaches a method of genetically engineering a pathogen resistance or tolerance trait in a plant, plant part, or plant cell, comprising providing a plant species that is susceptible to a pathogen, identifying within the genome of the plant species a homolog of FIT1, wherein said homolog does not mediate AvrFIT1 recognition; and genetically modifying a plant, plant part, or plant cell from the susceptible plant species with targeted gene editing, wherein said targeted gene editing is directed at the FIT1 homolog, and wherein said targeted gene editing enables the FIT1 homolog to recognize AvrFIT1 and confers resistance or tolerance to a pathogen.

The disclosure further relates to genetically modified plants, plant parts, or plant cells produced by the methods disclosed herein, wherein the plant, plant part, or plant cell exhibits resistance or tolerance to a pathogen. In some aspects, the pathogen is a fungus from the order Cantharellales or Pucciniales. In some aspects, the fungal pathogen is Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp. or Hemileia vastatrix. In some aspects, the plant, plant part, or plant cell is in the subfamily Papilionoideae. In some aspects, the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

The disclosure further relates to methods for identifying a functional FIT1 gene and/or allele thereof comprising isolating a FIT1 homolog or allele thereof, expressing said FIT1 homolog or allele thereof in combination with an effector protein produced by Phakopsora pachyrhizi in a plant, plant part, or plant cell; and assaying said plant, plant part, or plant cell for an immune response. In some aspects, the effector protein comprises SEQ ID NO: 24, SEQ ID: 26, or sequences at least 90% identical thereto.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 shows a phylogenetic tree of AvrFIT1 homologs identified by performing a BLAST® search of the NCBI protein database. Additional AvrFIT1 sequences were obtained from published Phakopsora pachyrhizi transcriptomes and proteomes. The obtained sequences were aligned using Clustal Omega and manually filtered to remove redundant and incomplete sequences. A maximum likelihood phylogenetic tree was constructed from the aligned protein sequences.

FIG. 2 shows photographs of plants inoculated with Phakopsora spores at two to four weeks of age. The images were taken at 14 to 21 days post inoculation. Presence of FIT1 in the plant genome correlates with resistance to ASR.

FIG. 3A shows a phylogenetic tree of homologs of Vigna unguiculata Vu01g041300.1 (VuFIT1) (SEQ ID NO: 2) identified by performing a BLAST® search and conducting a protein alignment. This figure shows putative FIT1 orthologs in Vigna radiata XP_014501487.1 (SEQ ID NO: 4), Vigna angularis XP_017423114.1 (SEQ ID NO: 12), Phaseolus acutifolius 005G036500.1 (SEQ ID NO: 10), Phaseolus lunatus P105g0000042900.1 (SEQ ID NO: 6), Phaseolus vulgaris PvUI111.05g037200.1 (SEQ ID NO: 8), Lablab purpureus c22535_g1_i3 (SEQ ID NO: 14), Mucuna pruriens RDX66260.1 (SEQ ID NO: 16), Cajanus cajan XP_020211972.1 (SEQ ID NO: 18), and Abrus precatorius XP_027357710.1 (SEQ ID NO: 20). Also shown is a paralog of FIT1 from Vigna unguiculata Vu01g041400.1 (SEQ ID NO: 22) believed to be the result of a recent duplication which accumulated mutations and is thus non-functional. The phylogenetic tree was rooted using paralogs of FIT1 that do not function in AvrFIT1 perception. Activity of each FIT1 homolog to function for recognition of AvrFIT1a and AvrFIT1b, based on transient assays, is indicated. nt=not tested.

FIG. 3B shows a protein alignment of the amino acid sequences listed in FIG. 3A, specifically of the Vigna unguiculata allele of FIT1 (VuFIT1) (SEQ ID NO: 2), the Vigna unguiculata close paralog of FIT1 (VuFIT1b) (SEQ ID NO: 22), Vigna angularis allele of FIT1 (VaFIT1) (SEQ ID NO: 12), the Vigna radiata allele of FIT1 (VrFIT1) (SEQ ID NO: 4), the Phaseolus acutifolius allele of FIT1 (PaFIT1) (SEQ ID NO: 10), the Phaseolus vulgaris allele of FIT1 (PvFIT1) (SEQ ID NO: 8), the Phaseolus lunatus allele of FIT1 (PlFIT1) (SEQ ID NO: 6), the Lablab purpureus allele of FIT1 (LpFIT1) (SEQ ID NO: 14), the Cajanus cajun allele of FIT1 (CcFIT1) 1 (SEQ ID NO: 18), the Mucuna pruriens allele of FIT1 (MpFIT1) (SEQ ID NO: 16), and the Abrus precatorius allele of FIT1 (ApFIT1) (SEQ ID NO: 20).

FIG. 3C shows a phylogenetic tree of homologs of Vigna unguiculata FIT1 in Glycine identified by performing a BLAST® search and constructing a protein alignment and phylogenetic tree of the resulting sequences. The tree was rooted using an outgroup of distantly related TIR-NLR proteins from non-legumes.

FIG. 4 shows a map of an example DNA construct comprising VuFIT1 (SEQ ID NO: 1) that can be used for transformation of a plant cell, selection of transformed cells, and expression of FIT1.

FIGS. 5A-5D depict results showing transient expression of various FIT1 alleles in leaf tissue from a plant lacking an endogenous FIT1. FIG. 5A shows the Vigna unguiculata allele of FIT1 (VuFIT1), the Phaseolus lunatus allele of FIT1 (PlFIT1), the Vigna radiata allele of FIT1 (VrFIT1), the Vigna unguiculata close paralog of FIT1 (VuFIT1b), and AvrFIT1a. FIG. 5B shows Vigna angularis allele of FIT1 (VaFIT1), the Phaseolus vulgaris allele of FIT1 (PvFIT1), the Lablab purpureus allele of FIT1 (LpFIT1), the Abrus precatorius allele of FIT1 (ApFIT1), and AvrFIT1a. FIG. 5C shows Vigna unguiculata allele of FIT1 (VuFIT1), the Phaseolus lunatus allele of FIT1 (PlFIT1), the Vigna radiata allele of FIT1 (VrFIT1), the Vigna unguiculata allele of FIT1 (VuFIT1b), and AvrFIT1b. FIG. 5D shows Vigna angularis allele of FIT1 (VaFIT1), the Phaseolus vulgaris allele of FIT1 (PvFIT1), the Lablab purpureus allele of FIT1 (LpFIT1), the Abrus precatorius allele of FIT1 (ApFIT1), and AvrFIT1b.

FIGS. 6A-6H depict wild type soybean leaves (lacking FIT1) (FIG. 6A-6D) and leaves from soybean plants expressing FIT1 (FIG. 6E-6H) inoculated with Phakopsora pachyrhizi. The leaves were photographed at 13- and 44-days post inoculation as indicated.

FIGS. 7A-7B are photographs of wild type soybean plants (FIG. 7A) and transgenic soybean plants expressing FIT1 (FIG. 7B).

FIG. 7C is a bar graph of the height of the plants shown in FIG. 9A and FIG. 9B 24 days after planting. The error bars indicate the standard deviation of the plant height from the individual plants (n>8).

FIG. 8 depicts an alignment between the FIT1 alleles from Vigna unguiculata (VuFIT1), Phaseolus lunatus (PlFIT1), Abrus precatorius (ApFIT1), and the N gene, which gives TMV resistance. The LRR domain is poorly conserved between the FIT1 alleles and the N gene.

### DETAILED DESCRIPTION

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of ±10% from the specified amount, as such variations are appropriate to perform the disclosed methods and/or employ the disclosed compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one or more of the elements to which the “and/or” refers can also individually be present in single or multiple occurrences in the combinations(s) and/or subcombination(s).

As used herein, the term “plant” can refer to any living organism belonging to the kingdom Plantae (i.e., any genus/species in the Plant Kingdom), to a whole plant, any part thereof, or a cell or tissue culture derived from a plant. Thus, the term “plant” can refer to any of whole plants, plant components or organs (e.g., leaves, stems, roots, etc.), plant tissues, seeds and/or plant cells.

A plant cell is a cell of a plant, taken from a plant, or derived through culture from a cell taken from a plant. Thus, the term “plant cell” includes without limitation cells within seeds, suspension cultures, embryos, meristematic regions, callus tissue, leaves, shoots, gametophytes, sporophytes, pollen, and microspores.

The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “resistant”, or “resistance”, describes a plant, line or variety that shows fewer or reduced symptoms to a biotic pest or pathogen than a susceptible (or more susceptible) plant, line or variety to that biotic pest or pathogen. This term is also applied to plants that show no symptoms, and may also be referred to as “high/standard resistance”.

As used herein, the term “tolerant” or “tolerance” describes a plant, line, or variety that that shows some symptoms to a biotic pest or pathogen, but that are still able to produce marketable product with an acceptable yield. These lines may also be referred to as having “moderate/intermediate resistance”. Tolerant and moderate/intermediate resistant plant types restrict the growth and development of the specified pest or pathogen, but exhibit a greater range of symptoms or damage compared to plant types with high resistance. Plant types with intermediate resistance will show less severe symptoms than susceptible plant varieties, when grown under similar field conditions and pathogen pressure. A “tolerant” plant may also indicate a phenotype of a plant wherein disease-symptoms remain absent upon exposure of said plant to an infective dosage of pathogen, whereby the presence of a systemic or local pathogen infection, pathogen multiplication, at least the presence of pathogen genomic sequences in cells of said plant and/or genomic integration thereof can be established. Tolerant plants are therefore resistant for symptom expression but symptomless carriers of the pathogen. Sometimes, pathogen sequences may be present or even multiply in plants without causing disease symptoms. This phenomenon is also known as “latent infection”. In latent infections, the pathogen may exist in a truly latent non-infectious occult form, possibly as an integrated genome or an episomal agent (so that pathogen protein cannot be found in the cytoplasm, while PCR protocols may indicate the present of pathogen nucleic acid sequences) or as an infectious and continuously replicating agent. A reactivated pathogen may spread and initiate an epidemic among susceptible contacts. The presence of a “latent infection” is indistinguishable from the presence of a “tolerant” phenotype in a plant.

Methods of evaluating resistance are well known to one skilled in the art. Such evaluation may be performed by visual observation of a plant or a plant part (e.g., leaves, roots, flowers, fruits et. al) in determining the severity of symptoms. For example, when each plant is given a resistance score on a scale of 1 to 5 based on the severity of the reaction or symptoms, with 1 being the resistance score applied to the most resistant plants (e.g., no symptoms, or with the least symptoms), and 5 the score applied to the plants with the most severe symptoms, then a line is rated as being resistant when at least 75% of the plants have a resistance score at a 1, 2, or 3 level, while susceptible lines are those having more than 25% of the plants scoring at a 4 or 5 level. If a more detailed visual evaluation is possible, then one can use a scale from 1 to 10 so as to broaden out the range of scores and thereby hopefully provide a greater scoring spread among the plants being evaluated. Additional methods for evaluating resistance are well known in the art (see for example, jircas.go.jp/sites/default/files/publication/manual_guideline/manual_guideline-_-_73.pdf available on the world wide web).

In addition to such visual evaluations, disease evaluations can be performed by determining the pathogen bio-density in a plant or plant part using electron and/or light microscopy and/or through molecular biological methods, such as protein quantification (e.g., ELISA, measuring pathogen protein density) and/or nucleic acid quantification (e.g., RT-PCR, measuring pathogen RNA density). Another method relies on quantifying the spores produced by the pathogen, which can be quantified using a hemacytometer and evaluated per uredinium, per leaf area, or per leaf.

As used herein, the term “susceptible” is used herein to refer to a plant that is unable to prevent entry of the pathogen into the plant and/or slow multiplication and systemic spread of the pathogen, resulting in disease symptoms. The term “susceptible” is therefore equivalent to “non-resistant”.

As used herein, the term “homologous” or “homolog” is used as it is known in the art and refers to related sequences that share a common ancestor. The term “homolog” is sometimes used to apply to the relationship between genes separated by the event of speciation (“ortholog”) or to the relationship between genes separated by the event of genetic duplication within the same species (“paralog”). Homology can be determined using software programs readily available in the art, such as those discussed in Current Protocols in Molecular Biology (F. M. Ausubel et al., eds., 1987) Supplement 30, section 7.718, Table 7.71.

As used herein, the term “allele” is used both as it is known in the art as one of two or more versions of a gene or peptide, and also to refer to synthetic variants of a gene or peptide containing one or more changes from the native sequence.

As used herein, the term “functional” used in the context of a homolog means that the homolog has the same or very similar function. For example, a functional homolog of FIT1 would recognize an AvrFIT1 effector protein. A “nonfunctional FIT1 homolog” would not recognize AvrFIT1, though it may still be functional in that it is able recognize other effector proteins.

As used herein, the term “sequence identity” refers to the presence of identical nucleotides or amino acids at corresponding positions of two sequences. Readily available sequence comparison and multiple sequence alignment algorithms are, respectively, the Basic Local Alignment Search Tool (BLAST®) and ClustalW/ClustalW2/Clustal Omega programs available on the Internet (e.g., the website of the EMBL-EBI). Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001. Unless otherwise noted, alignments disclosed herein utilized Clustal Omega.

As used herein, the phrases “DNA construct”, “expression cassette”, “chimeric construct”, “construct”, and “recombinant DNA construct” are used interchangeably herein. A recombinant DNA construct comprises an artificial combination of nucleic acid fragments, e.g., regulatory and coding sequences that are not found together in nature. For example, a construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746).

As used herein “cisgene” refers to a gene from the same species, or a species closely related enough to be conventionally bred. “Transgene” refers to a gene from a different species, and may also be referred to as “heterologous” (an amino acid or a nucleic acid sequence which is not naturally found in the particular organism). Both transgenes and heterologous sequences would be considered “exogenous” as referring to a substance coming from some source other than its native source.

The term “operably linked” refers to the juxtaposition of two or more components (such as sequence elements) having a functional relationship. For example, the sequential arrangement of the promoter polynucleotide with a further oligo- or polynucleotide, resulting in transcription of the further polynucleotide.

As used herein, “promoter” refers to a DNA sequence capable of controlling the expression of a coding sequence or functional RNA. In some embodiments, the promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter.

As used herein, “selectable marker” is a nucleic acid segment that allows one to select for a molecule (e.g., a plasmid) or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

The following description includes information that may be useful in understanding the present disclosure. It is not an admission that any of the information provided herein is prior art or relevant to the presently claimed disclosures, or that any publication specifically or implicitly referenced is prior art.

### Overview

The present disclosure provides an isolated, recombinant, or synthetic polynucleotide comprising a FIT1 protein, and homologs, fragments, and variations thereof. The disclosure further relates to plants, plant parts, and plant cells that have been transformed with these polynucleotides, and exhibit resistance or tolerance to a plant pathogen, such as Phakopsora pachyrhizi. The disclosure further relates to methods of identifying pathogen resistant genes, and methods of genetically engineering a pathogen resistance or tolerance trait in a plant, plant part, or plant cell, comprising targeted gene editing of a homolog (such as FIT1), and plants produced therefrom.

### Plant Pathogens

There are more than 6000 species of rust fungi, including for example, Phakopsora pachyrhizi, Puccinia spp., Uromyces appendiculatus, and Hemileia vastatrix, that infect a wide range of important crops and ornamental varieties. Some examples of varieties that may be infected include, but are not limited to, Avena sativa (oats), Vicia faba (broad beans), Coffea arabica (coffee), Chrysanthemum, Cydonia (quince), Fuchsia spp. (garlic), Hordeum vulgare (barley), Juniperus virginiana (red cedar), Juniperus communis (juniper), Allium ampeloprasum (leek), Malus spp. (apple), Mentha piperita (peppermint), Mespilus (medlar), Onion, Pelargonium, Primula vulgaris (primrose), Pyrus (pear), Rosa spp. (roses), Triticum spp. (wheat), Secale cereale (rye), Vitis vinfera (grape), Saccharum spp. (sugarcane) and Glycine max (soybean).

In soybean, rust pathogen infections have been reported in South America, Africa, Australia, and Asia, and were first reported on soybean crops in the southern United States in 2004. Asian soybean rust (ASR), caused by Phakopsora pachyrhizi, spreads quickly and can lead to significant yield loss. Initial symptoms of ASR include yellow discoloration on the upper surfaces of foliage, followed by tan or reddish-brown lesions on the undersides of the leaves and sometimes on petioles, stems or pods. Blisters develop within the lesions, which break open and release spores. Soybean plants infected with ASR will exhibit reduced pod production and can result in a yield loss of greater than 50%. Another disease, New World soybean rust, caused by Phakopsora meibomiae, is generally not as harmful as ASR. P. meibomiae has not yet been reported in the U.S.

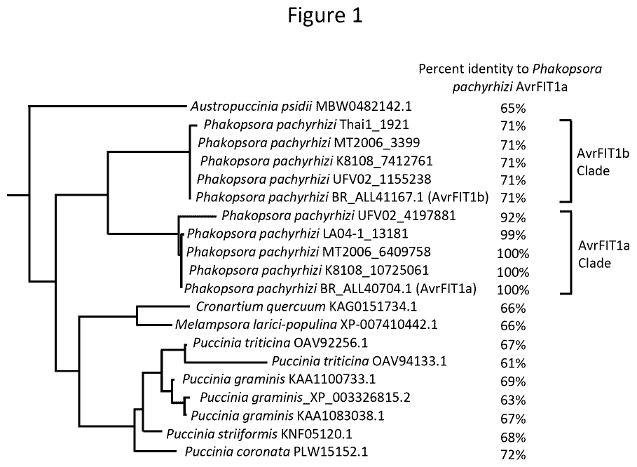

Successful infection of rust fungi relies on the secretion of effector proteins with functions that facilitate host colonization. The effector proteins suppress plant immunity and manipulate the host metabolism to benefit the pathogen (Jones and Dangl, 2006). AvrFIT1 is one such effector protein secreted by Phakopsora pachyrhizi that causes ASR. AvrFIT1 is present in several sequenced Phakopsora pachyrhizi strains isolated from various locations across the world, including Brazil and North America. A phylogenetic tree of close homologs of AvrFIT1 reveals that there are two similar copies of AvrFIT1 in Phakopsora pachyrhizi (FIG. 1), (PpAvrFIT1a and PpAvrFIT1b), both of which are recognized by FIT1 in transient assays. AvrFIT1 is also conserved in many other fungal species. Thai1, LA04-1 and MG2006 are three strains of Phakopsora pachyrhizi that all contain recognized alleles of AvrFIT1 (Elmore et al., 2020; Link et al., 2014, and information on Phakopsora pachyrhizi from the genome portal of the Department of Energy Joint Genome Institute available on the worldwide web.). Phakopsora pachyrhizi BR is an unspecified Brazilian population which also contains recognized alleles of AvrFIT1 (de Carvalho et al., 2017). AvrFIT1 is present in species of rust pathogens that cause disease on poplar (Melampsora larici-populina), cereals (Puccinia spp.) and other plants. Putative orthologs of AvrFIT1 are also present in more distantly related fungal species including both pathogenic and non-pathogenic fungi. For example, AvrFIT1 is present in Rhizoctonia solani, a non-rust pathogen that can be problematic for herbaceous plants, causing diseases such as collar rot, root rot, damping off, and wire stem.

AvrFIT1 is a putative peptidyl prolyl isomerase and is predicted to disrupt or perturb the function of one or more plant host proteins. While not wishing to be bound by any particular theory, it is possible that AvrFIT1 could be modifying an unknown plant protein, possibly a conserved component of the plant immune system, and FIT1 could be “guarding” the unknown protein and be activated if the protein is modified by AvrFIT1. There are many examples of NLR receptors acting this way in the literature. Pathogen effectors often convergently evolve to target the same or similar plant host proteins to promote virulence. There are many examples of two different pathogen effector proteins evolving to target the same plant protein independently. Thus, it is possible that another pathogen might have an effector protein that is unrelated to AvrFIT1 but acts on the same protein as AvrFIT1 and therefore is also capable of being perceived by FIT1. By guarding against proteins with a similar activity or molecular target as AvrFIT1, FIT1 can mediate resistance against pathogens which don't have a close homolog of AvrFIT1 but do have a protein which has evolved to have a similar activity or molecular target as FIT1. Such proteins may be present in a diverse range of plant pathogens including but not limited to fungi, oomycetes, bacteria, nematodes, or viruses.

### Plant Resistance

Plants have, in some cases, evolved immunity in which resistance gene products recognize the activity of specific effectors resulting in effector-trigger immunity (ETI) (Jones and Dangl, 2006). ETI leads to robust defenses, such as the hypersensitive response (HR), which is a form of programmed cell death that results in the formation of a localized lesion that inhibits pathogen growth at the initial infection site (Dodds and Rathjen, 2010). If the plant has an immune receptor capable of recognizing the pathogen effector protein, the effector protein activates a strong immune response conferring immunity. The perception of intracellular pathogen effector proteins in plants is frequently mediated by proteins from a large gene family known as the nucleotide binding, leucine-rich repeat (NLR) proteins (Jones et al., 2016).

Plant disease resistance traits are often encoded by NLR genes. NLR genes can be incorporated into a susceptible crop variety to confer resistance through a variety of methods including introgression breeding, transformation or genome editing. A typical plant has hundreds of NLR immune receptor genes (Jones et al., 2016). These genes are typically expressed at relatively low levels with the NLR proteins passively surveilling for the presence of cognate effector proteins from invading pathogens. Prior to activation, the NLR proteins have essentially no impact on plant metabolism or growth. Upon activation by a cognate ligand, typically a pathogen effector protein or a protein substrate of an effector, the NLR protein initiates a signalling cascade that activates endogenous plant defense pathways to inhibit pathogen growth. Using NLRs is a natural, safe, and environmentally sustainable mechanism to develop disease-resistant crop varieties to improve plant yields and reduce the need for chemical controls.

### FIT1

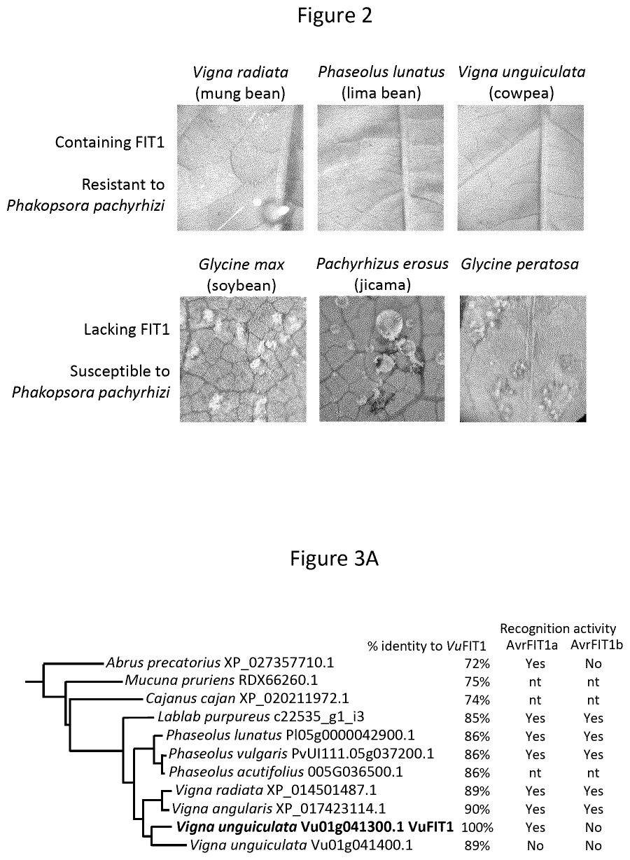

FIT1 is a plant Toll-like interleukin-1 receptor (TIR) nucleotide binding leucine rich repeat (NLR) immune receptor protein discovered by Applicants. It was identified from Vigna unguiculata (cowpea) and is responsible for AvrFIT1 recognition. As shown in FIG. 2, expression of FIT1 correlates with resistance to ASR. Accessions of Vigna radiata, (PI 378026), Phaseolus lunatus (PI 180324), and Vigna unguiculata (LG104, Baker Creek Heirloom Seeds) which contain functional copies of FIT1 genes show strong resistance to Phakopsora pachyrhizi. In contrast, accessions of Glycine max (Williams 82), Pachyrhizus erosus (AB105, Baker Creek Heirloom Seeds), and Glycine peratosa (PI 583964), which all lack functional FIT1, are highly susceptible to Phakopsora pachyrhizi as observed by large disease lesions and spore production. These results indicate a correlation between the presence of FIT1 and resistance to Phakopsora pachyrhizi and suggest that expression of FIT1 in plants can confer disease resistance. Additionally, the widespread distribution of AvrFIT1 discussed above suggests that FIT1 can confer disease resistance against a broad range of pathogenic species.

Thus, an embodiment of the present disclosure provides an isolated, recombinant, or synthetic polynucleotide comprising a nucleic acid sequence encoding a FIT1 protein, wherein the protein is selected from the group consisting of: SEQ ID NOs: 2, 4, 6, 8, 10, 12, 14, 16, 18, 20, functional homologs, and/or fragments and variations thereof. In some cases, the functional FIT1 homolog shares at least about 70% identity to SEQ ID NO: 2 and recognizes an effector protein secreted by a plant pathogen. In some cases, the functional FIT1 homolog recognizes at least one of AvrFIT1a and AvrFIT1b. In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 2.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 4.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 6.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 8.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 10.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 12

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 14.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 16.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 18.

In some cases, the functional FIT1 homolog shares at least about 70%, about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 20.

In some aspects, the isolated, recombinant, or synthetic polynucleotide comprises a nucleic acid sequence encoding SEQ ID NO: 2, or an amino acid sequence at least 90% identical thereto. In some aspects, the isolated, recombinant, or synthetic polynucleotide comprises a nucleic acid sequence encoding SEQ ID NO: 4, or an amino acid sequence at least 90% identical thereto. In some aspects, the isolated, recombinant, or synthetic polynucleotide comprises a nucleic acid sequence encoding SEQ ID NO: 6, or an amino acid sequence at least 90% identical thereto. In some aspects, the isolated, recombinant, or synthetic polynucleotide comprises a nucleic acid sequence encoding SEQ ID NO: 8, or an amino acid sequence at least 90% identical thereto. In some aspects, the isolated, recombinant, or synthetic polynucleotide comprises a nucleic acid sequence encoding SEQ ID NO: 10, or an amino acid sequence at least 90% identical thereto. In some aspects, the isolated, recombinant, or synthetic polynucleotide comprises a nucleic acid sequence encoding SEQ ID NO: 12, or an amino acid sequence at least 90% identical thereto. In some aspects, the isolated, recombinant, or synthetic polynucleotide comprises a nucleic acid sequence encoding SEQ ID NO: 14, or an amino acid sequence at least 90% identical thereto. In some aspects, the isolated, recombinant, or synthetic polynucleotide comprises a nucleic acid sequence encoding SEQ ID NO: 16, or an amino acid sequence at least 90% identical thereto. In some aspects, the isolated, recombinant, or synthetic polynucleotide comprises a nucleic acid sequence encoding SEQ ID NO: 18, or an amino acid sequence at least 90% identical thereto. In some aspects, the isolated, recombinant, or synthetic polynucleotide comprises a nucleic acid sequence encoding SEQ ID NO: 20, or an amino acid sequence at least 90% identical thereto.

In another embodiment, the disclosure relates to a transgenic plant, plant part, or cell having resistance or tolerance to at least one plant pathogen, wherein the resistance or tolerance is conferred by a polynucleotide encoding at least one of the functional FIT1 homologs disclosed herein.

In another embodiment, the present disclosure provides an isolated, recombinant, or synthetic polynucleotide, wherein the polynucleotide comprises a nucleic acid sequence selected from the group consisting of SEQ ID NOs: 1, 3, 5, 7, 9, 11, 13, 15, 17, 19 complements thereof, fragments thereof, and sequences at least 70% identical thereto, wherein said sequences encode a functional FIT1 protein. In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 1. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 3. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 5. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 7. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 9. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 11. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 13. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 15. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 17. In some embodiments, the disclosure relates to genetic constructs comprising these sequences.

In some cases, the polynucleotide shares at least about 70%, at least about 71%, about 72%, about 73%, about 74%, about 75%, about 76%, about 77%, about 78%, about 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 19. In some embodiments, the disclosure relates to genetic constructs comprising these sequences.

The disclosure also encompasses variants and fragments of proteins of an amino acid sequence encoded by the nucleic acid sequences of FIT1, orthologs of FIT1 and/or paralogs of FIT1. The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both.

Functional fragments and variants of a polypeptide include those fragments and variants that maintain one or more functions or domains of the parent polypeptide. As used herein, a protein domain is a distinct functional and/or structural unit in a protein, and are usually responsible for a particular function or interaction. It is recognized that the gene or cDNA encoding a polypeptide can be considerably mutated without materially altering one or more of the polypeptide's functions and/or domains. First, the genetic code is well-known to be degenerate, and thus different codons encode the same amino acids. Second, even where an amino acid substitution is introduced, the mutation can be conservative and have no material impact on the essential function(s) of a protein. See, e.g., Stryer Biochemistry 3^{rd} Ed., 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al. J. Immunol. 159(5): 2502-12, 1997). Other modifications that can be made without materially impairing one or more functions of a polypeptide can include, for example, in vivo or in vitro chemical and biochemical modifications or the incorporation of unusual amino acids. Such modifications include, but are not limited to, for example, acetylation, carboxylation, phosphorylation, glycosylation, ubiquination, labelling, e.g., with radionucleotides, and various enzymatic modifications, as will be readily appreciated by those well skilled in the art. A variety of methods for labelling polypeptides, and labels useful for such purposes, are well known in the art, and include radioactive isotopes such as ³²P, ligands which bind to or are bound by labelled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues. These mutations can be natural or purposely changed. In some embodiments, mutations containing alterations that produce silent substitutions, additions, or deletions, but do not alter the properties or activities of the proteins or how the proteins are made are an embodiment of the present disclosure.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio Technology, 6:1321-1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table 1 shows exemplary conservative amino acid substitutions.

TABLE 1

| | Very Highly - | Highly Conserved | Conserved |
|---|---|---|---|
| Original | Conserved | Substitutions (from the | Substitutions (from the |
| Residue | Substitutions | Blosum90 Matrix) | Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, |
| | | | Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, |
| | | | Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

### Codon Optimization

In some cases, variants may differ from the disclosed sequences by alteration of the coding region to fit the codon usage bias of the particular organism into which the molecule is to be introduced. In other cases, the coding region may be altered by taking advantage of the degeneracy of the genetic code to alter the coding sequence such that, while the nucleotide sequence is substantially altered, it nevertheless encodes a protein having an amino acid sequence substantially similar to the disclosed amino acid sequences of FIT1, orthologs of FIT1 and/or paralogs of FIT1, and/or fragments and variations thereof.

Protein expression is governed by a host of factors including those that affect transcription, mRNA processing, and stability and initiation of translation. Optimization can thus address any of a number of sequence features of any particular gene. Translation may be paused due to the presence of codons in the polynucleotide of interest that are rarely used in the host organism, and this may have a negative effect on protein translation due to their scarcity in the available tRNA pool. Specifically, it can result in reduced protein expression.

Alternate translational initiation also can result in reduced heterologous protein expression. Alternate translational initiation can include a synthetic polynucleotide sequence inadvertently containing motifs capable of functioning as a ribosome binding site (RBS). These sites can result in initiating translation of a truncated protein from a gene-internal site. One method of reducing the possibility of producing a truncated protein includes eliminating putative internal RBS sequences from an optimized polynucleotide sequence.

Repeat-induced polymerase slippage can result in reduced heterologous protein expression. Repeat-induced polymerase slippage involves nucleotide sequence repeats that have been shown to cause slippage or stuttering of DNA polymerase which can result in frameshift mutations. Such repeats can also cause slippage of RNA polymerase. In an organism with a high G+C content bias, there can be a higher degree of repeats composed of G or C nucleotide repeats. Therefore, one method of reducing the possibility of inducing RNA polymerase slippage, includes altering extended repeats of G or C nucleotides.

Interfering secondary structures also can result in reduced heterologous protein expression. Secondary structures can sequester the RBS sequence or initiation codon and have been correlated to a reduction in protein expression. Stemloop structures can also be involved in transcriptional pausing and attenuation. An optimized polynucleotide sequence can contain minimal secondary structures in the RBS and gene coding regions of the nucleotide sequence to allow for improved transcription and translation.

The optimization process can begin, for example, by identifying the desired amino acid sequence to be expressed by the host. From the amino acid sequence, a candidate polynucleotide or DNA sequence can be designed. During the design of the synthetic DNA sequence, the frequency of codon usage can be compared to the codon usage of the host expression organism and rare host codons can be removed from the synthetic sequence. Additionally, the synthetic candidate DNA sequence can be modified in order to remove undesirable enzyme restriction sites and add or remove any desired signal sequences, linkers or untranslated regions. The synthetic DNA sequence can be analyzed for the presence of secondary structure that may interfere with the translation process, such as G/C repeats and stem-loop structures.

Optimized coding sequences containing codons preferred by a particular host can be prepared, for example, to increase the rate of translation or to produce recombinant RNA transcripts having desirable properties, such as a longer half-life, as compared with transcripts produced from a non-optimized sequence.

### Functional Fragments, Chimeric, and Synthetic Polypeptides

In some cases, functional fragments derived from FIT1 orthologs of the present disclosure can still confer resistance to pathogens when expressed in a plant. In some cases, the functional fragments contain one or more conserved regions shared by two or more FIT1 orthologs.

In some cases, functional chimeric or synthetic polypeptides derived from the FIT1 orthologs of the present disclosure are provided. The functional chimeric or synthetic polypeptides can still confer resistance to pathogens when expressed in a plant. In some cases, the functional chimeric or synthetic polypeptides contain one or more conserved regions shared by two or more FIT1 orthologs.

### DNA Constructs

In some embodiments, the disclosure relates to a DNA construct comprising at least one FIT1 sequence disclosed herein. In some cases, the FIT1 sequence is a polynucleotide comprising a nucleic acid sequence encoding a FIT1 protein, wherein the protein is selected from the group consisting of: SEQ ID NOs: 2, 4, 6, 8, 10, 12, 14, 16, 18, 20, functional homologs, and/or fragments and variations thereof. In some cases, the FIT1 protein shares at least about 70% identity to SEQ ID NO: 2. In some cases, the FIT1 sequence is selected from SEQ ID NOs: 1, 3, 5, 7, 9, 11, 13, 15, 17, 19 complements thereof, fragments thereof, and sequences at least 70% identical thereto. In some cases, two or more FIT1 sequences are stacked to increase pathogen resistance in a plant. In some cases, at least one FIT1 sequence is stacked with another pathogen resistance gene.

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make DNA constructs for use in the present disclosure. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Lastly, plant promoters such as prolifera promoter, fruit specific promoters, Ap3 promoter, heat shock promoters, seed specific promoters, etc. can also be used.

Either a gamete-specific promoter, a constitutive promoter (such as the CaMV or Nos promoter), an organ-specific promoter (such as the E8 promoter from tomato), or an inducible promoter is typically ligated to the protein or antisense encoding region using standard techniques known in the art. The DNA construct may be further optimized by employing supplemental elements such as transcription terminators and/or enhancer elements.

Thus, for expression in plants, the DNA construct will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a pre-existing vector.

In the construction of heterologous promoter/gene of interest or antisense combinations, the promoter is preferably positioned about the same distance from the heterologous transcription start site as it is from the transcription start site in its natural setting. As is known in the art, however, some variation in this distance can be accommodated without loss of promoter function.

In addition to a promoter sequence, the expression cassette can also contain a transcription termination region downstream of the gene to provide for efficient termination. The termination region may be obtained from the same gene as the promoter sequence or may be obtained from different genes. If the mRNA encoded by the gene is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835-846 (1984)) or the nopaline synthase signal (Depicker et al., Mol. and Appl. Genet. 1:561-573 (1982)). The resulting expression unit is ligated into or otherwise constructed to be included in a vector that is appropriate for higher plant transformation. One or more expression units may be included in the same vector.

### Selection

A DNA construct will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, are generally also included to allow the vector to be cloned in a bacterial or phage host; preferably a broad host range for prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector, as is known in the art. For instance, in the case of Agrobacterium transformations, T-DNA sequences will also be included for subsequent transfer to plant chromosomes.

For positive selection, for example, a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of non-transformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631(1990), U.S. Pat. Nos. 4,795,855, 5,378,824 and 6,107,549). In addition, the dhfr gene, which confers resistance to the anticancer agent methotrexate, has been used for selection (Bourouis et al., EMBO J. 2(7): 1099-1104 (1983).

### Transgenic Plants Comprising Sequences Disclosed Herein

In one embodiment, the present disclosure relates to a transgenic plant, plant part, or plant cell, wherein the transgene comprises at least one polynucleotide coding for FIT1, orthologs of FIT1 and/or paralogs of FIT1, and/or fragments and variations thereof, and exhibit resistance or tolerance to a pathogen. In some cases, the polynucleotide encodes a protein selected from the group consisting of: SEQ ID NOs: 2, 4, 6, 8, 10, 12, 14, 16, 18, 20, and proteins at least 90% identical thereto functional homologs thereof. In some cases, the polynucleotide comprises a nucleic acid sequence selected from the group consisting of SEQ ID NOs: 1, 3, 5, 7, 9, 11, 13, 15, 17, 19 complements thereof, fragments thereof, and sequences at least 70% identical thereto. In some cases, the pathogen is a fungus. In some cases, the fungus is from the order Cantharellales or Pucciniales. In some cases, the fungal pathogen is Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp. or Hemileia vastatrix.

In some cases, the plant, plant part, or plant cell is in the subfamily Papilionoideae. In some cases, the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

In some cases, the plant, plant part, or plant cell is Glycine max, and the plant, plant part, or plant cell is resistant to Asian Soybean Rust caused by Phakopsora pachyrhizi.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

Microprojectile bombardment is also known as particle acceleration, biolistic bombardment, and the gene gun (Biolistic® Gene Gun). The gene gun is used to shoot pellets that are coated with genes (e.g., for desired traits) into plant seeds or plant tissues in order to get the plant cells to then express the new genes. The gene gun uses an actual explosive (.22 caliber blank) to propel the material. Compressed air or steam may also be used as the propellant. The Biolistic® Gene Gun was invented in 1983-1984 at Cornell University by John Sanford, Edward Wolf, and Nelson Allen. It and its registered trademark are now owned by E. I. du Pont de Nemours and Company. Most species of plants can be been transformed using this method.

The most common method for the introduction of new genetic material into a plant genome involves the use of living cells of the bacterial pathogen Agrobacterium tumefaciens. Agrobacterium tumefaciens is a naturally occurring bacterium that is capable of inserting its DNA (genetic information) into plants, resulting in a type of injury to the plant known as crown gall. Most species of plants can now be transformed using this method. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Methods of Agrobacterium-mediated plant transformation that involve using vectors with no T-DNA are also well known to those skilled in the art and can be used with the methods of the present disclosure. See, for example, U.S. Pat. No. 7,250,554, which utilizes P-DNA instead of T-DNA in the transformation vector. A transgenic plant formed using Agrobacterium transformation methods typically contains a single gene on one chromosome, although multiple copies are possible. Such transgenic plants can be referred to as being hemizygous for the added gene, or may be referred to as an independent segregant, because each transformed plant represents a unique T-DNA integration event (U.S. Pat. No. 6,156,953).

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988).

Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope (U.S. Pat. Nos. 5,204,253, 5,015,580).

A third direct method uses fibrous forms of metal or ceramic consisting of sharp, porous or hollow needle-like projections that impale the cells, and also the nuclear envelope of cells. Both silicon carbide and aluminium borate whiskers have been used for plant transformation (Mizuno et al., 2004; Petolino et al., 2000; U.S. Pat. No. 5,302,523 U.S. Application Publication No. 2004/0197909) and also for bacterial and animal transformation (Kaepler et al., 1992; Raloff, 1990; Wang, 1995).

Examples of viral vectors include, but are not limited to, recombinant plant viruses. Non-limiting examples of plant viruses include, TMV-mediated (transient) transfection into tobacco (Tuipe, T-H et al (1993), J. Virology Meth, 42: 227-239), ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae). Detailed classification information of plant viruses can be found in Fauquet et al (2008, “Geminivirus strain demarcation and nomenclature”. Archives of Virology 153:783-821, incorporated herein by reference in its entirety), and Khan et al. (Plant viruses as molecular pathogens; Publisher Routledge, 2002, ISBN 1560228954, 9781560228950). Examples of non-viral vectors include, but are not limited to, liposomes, polyamine derivatives of DNA, and the like.

Non-limiting examples of binary vectors suitable for soybean species transformation and transformation methods are described by Yi et al. 2006 (Transformation of multiple soybean cultivars by infecting cotyledonary-node with Agrobacterium tumefaciens, African Journal of Biotechnology Vol. 5 (20), pp. 1989-1993, 16 Oct. 2006), Paz et al., 2004 (Assessment of conditions affecting Agrobacterium-mediated soybean transformation using the cotyledonary node explant, Euphytica 136: 167-179, 2004), U.S. Pat. Nos. 5,376,543, 5,416,011, 5,968,830, and 5,569,834, or by similar experimental procedures well known to those skilled in the art.

Genes can also be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

### Genetically Engineering a Pathogen Resistance or Tolerance Trait in a Plant, Plant Part, or Plant Cell

An embodiment of the present disclosure teaches a method of genetically engineering a pathogen resistance or tolerance trait in a plant, plant part, or plant cell, comprising: providing a plant species that is susceptible to a pathogen; identifying within the genome of the plant species a homolog of FIT1, wherein said homolog is nonfunctional (does not mediate AvrFIT1 recognition), and genetically modifying a plant, plant part, or plant cell from the susceptible plant species with targeted gene editing, wherein said targeted gene editing is directed towards the nonfunctional FIT1 homolog, and wherein said targeted gene editing restores the function of FIT1 (enables the FIT1 homolog to recognize AvrFIT1) and confers resistance or tolerance to a pathogen. In some embodiments, the pathogen is a fungus.

As used herein, a “nonfunctional” FIT1 homolog is a homolog that does not recognize a pathogen effector protein homolog of AvrFIT1, such as AvrFIT1a and/or AvrFIT1b. FIT1 homologous may identified by any number of means known in the art. Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

Restoring the function of a FIT1 homolog as used herein relates to modifying the allele such that it restores the recognition of a pathogen effector protein such as AvrFIT1a and/or AvrFIT1b, and confers resistance or tolerance to a pathogen. Restoring the function of a homologous gene by way of genetic engineering has been done and is well known in the art (see for example, Ivics Z, et al., (1997), “Molecular reconstruction of Sleeping Beauty, a Tc1-like transposon from fish, and its transposition in human cells”, Cell. 91 (4): 501-510, and recently, Suh S, et al., Restoration of visual function in adult mice with an inherited retinal disease via adenine base editing, Nat Biomed Eng (2020) and Sedeek K, et al., Plant genome engineering for targeted improvement of crop traits, Front. Plant Sci, 12 Feb. 2019).

In some embodiments, the targeted gene editing uses an engineered or natural nuclease selected from the group consisting of homing endonucleases/meganucleases (EMNs), zinc finger nucleases (ZFNs), and transcription activator-like effector nucleases (TALENs). In some embodiments, the targeted gene editing uses a clustered regularly interspaced short palindromic repeats (CRISPR)-Cas nuclease. In some embodiments, the nuclease is selected from the group consisting of Cas9, Cas12, Cas13, CasX, and CasY. The disclosure also relates to plants, plant parts, and plant cells exhibiting resistance or tolerance to a pathogen produced by genetic modification of a FIT1 homolog.

### Gene Editing Using CRISPR

Targeted gene editing can be done using CRISPR technology (Saunders & Joung, Nature Biotechnology, 32, 347-355, 2014). CRISPR is a type of genome editing system that stands for Clustered Regularly Interspaced Short Palindromic Repeats. This system and CRISPR-associated (Cas) genes enable organisms, such as select bacteria and archaea, to respond to and eliminate invading genetic material. Ishino, Y., et al. J. Bacteriol. 169, 5429-5433 (1987). These repeats were known as early as the 1980s in E. coli, but Barrangou and colleagues demonstrated that S. thermophilus can acquire resistance against a bacteriophage by integrating a fragment of a genome of an infectious virus into its CRISPR locus. Barrangou, R., et al. Science 315, 1709-1712 (2007). Many plants have already been modified using the CRISPR system, including soybean (see for example, Han J, et al., Creation of early flowering germplasm of soybean by CRISPR/Cas9 Technology, Front. Plant Sci., 22 Nov. 2019), and many Cas genes have now been characterized and used with the system (see for example, Wang J, et al., The rapidly advancing Class 2 CRISPR-Cas technologies: A customizable toolbox for molecular manipulations. J Cell Mol Med. 2020; 24(6):3256-3270).

Gene editing can also be done using crRNA-guided surveillance systems for gene editing. Additional information about crRNA-guided surveillance complex systems for gene editing can be found in the following documents, which are incorporated by reference in their entirety: U.S. Application Publication No. 2010/0076057 (Sontheimer et al., Target DNA Interference with crRNA); U.S. Application Publication No. 2014/0179006 (Feng, CRISPR-CAS Component Systems, Methods, and Compositions for Sequence Manipulation); U.S. Application Publication No. 2014/0294773 (Brouns et al., Modified Cascade Ribonucleoproteins and Uses Thereof); Sorek et al., Annu. Rev. Biochem. 82:237-266, 2013; and Wang, S. et al., Plant Cell Rep (2015) 34: 1473-1476.

### Gene Editing Using TALENs

Transcription activator-like effector nucleases (TALENs) have been successfully used to introduce targeted mutations via repair of double stranded breaks (DSBs) either through non-homologous end joining (NHEJ), or by homology-directed repair (HDR) and homology-independent repair in the presence of a donor template. Thus, TALENs are another mechanism for targeted genome editing in plants. The technique is well known in the art; see for example Malzahn, Aimee et al. “Plant genome editing with TALEN and CRISPR” Cell & Bioscience vol. 7 21. 24 Apr. 2017.

### Other Methods of Genome Editing

In addition to CRISPR and TALENs, two other types of engineered nucleases can be used for genome editing: engineered homing endonucleases/meganucleases (EMNs), and zinc finger nucleases (ZFNs). These methods are well known in the art. See for example, Petilino, Joseph F. “Genome editing in plants via designed zinc finger nucleases” In Vitro Cell Dev Biol Plant. 51(1): pp. 1-8 (2015); and Daboussi, Fayza, et al. “Engineering Meganuclease for Precise Plant Genome Modification” in Advances in New Technology for Targeted Modification of Plant Genomes. Springer Science+Business. pp 21-38 (2015).

### Breeding Methods

Once a gene has been introduced into a plant, or a gene has been genetically modified, that plant can then be used in conventional plant breeding schemes (e.g., pedigree breeding, single-seed-descent breeding schemes, recurrent selection, backcross breeding) to produce progeny which also contain the gene or modified trait. Thus, another aspect of the present disclosure relates to breeding with, or asexually propagating, plants having been transformed with a FIT1 homolog or an immune receptor gene coding for a protein that recognizes AvrFIT1a and/or AvrFIT1b, or plants wherein a FIT1 nonfunctional homolog was genetically modified to recognize AvrFIT1a and/or AvrFIT1b, wherein the plants exhibit resistance or tolerance to a pathogen. The disclosure further relates to progeny plants produced therefrom.

In some cases, plants or progeny therefrom comprising the gene or modified trait may further comprise one or more additional desired traits. In some cases, the one or more additional desired traits are stacked on the same construct as the gene (for example, the FIT1 genes disclosed herein). In another case, the one or more additional desired traits may be introgressed by conventional breeding.

### Backcross Breeding

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent. As used herein, backcross breeding is synonymous with introgression. Plants produced therefrom may be referred to a single locus converted or single gene converted plants.

A non-limiting example of a backcross breeding protocol would be the following: a) the first generation F₁ produced by the cross of the recurrent parent A by the donor parent B is backcrossed to parent A, b) selection is practiced for the plants having the desired trait of parent B, c) selected plants are self-pollinated to produce a population of plants where selection is practiced for the plants having the desired trait of parent B and physiological and morphological characteristics of parent A, d) the selected plants are backcrossed one, two, three, four, five, six, seven, eight, nine, or more times to parent A to produce selected backcross progeny plants comprising the desired trait of parent B and the physiological and morphological characteristics of parent A. Step (c) may or may not be repeated and included between the backcrosses of step (d).

Examples of desired traits include, but are not limited to, herbicide resistance (such as bar or pat genes), resistance for bacterial, fungal, or viral disease (such as gene I used for BCMV resistance), insect resistance, enhanced nutritional quality (such as 2s albumin gene), industrial usage, agronomic qualities (such as the “persistent green gene”), yield stability, and yield enhancement.

### Pedigree Selection

Pedigree breeding is used commonly for the improvement of self-pollinating crops or inbred lines of cross-pollinating crops. Two parents possessing favorable, complementary traits are crossed to produce an F₁. An F₂ population is produced by selfing one or several F₁s or by intercrossing two F₁s (sib mating). The dihaploid breeding method could also be used. Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release of new cultivars. Similarly, the development of new cultivars through the dihaploid system requires the selection of the cultivars followed by two to five years of testing in replicated plots.

### Open-Pollination

The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988). For population improvement methods specific for soybean see, e.g., J. R. Wilcox, editor (1987) SOYBEANS: Improvement, Production, and Uses, Second Edition, American Society of Agronomy, Inc., Crop Science Society of America, Inc., and Soil Science Society of America, Inc., publishers, 888 pages.

### Hand-Pollination Method

Hand pollination describes the crossing of plants via the deliberate fertilization of female ovules with pollen from a desired male parent plant. In some cases the donor or recipient female parent and the donor or recipient male parent line are planted in the same field or in the same greenhouse. The inbred male parent can be planted earlier than the female parent to ensure adequate pollen supply at the pollination time. Pollination is started when the female parent flower is ready to be fertilized. Female flower buds that are ready to open in the following days are identified, covered with paper cups or small paper bags that prevent bee or any other insect from visiting the female flowers, and marked with any kind of material that can be easily seen the next morning. The male flowers of the male parent are collected in the early morning before they are open and visited by pollinating insects. The covered female flowers of the female parent, which have opened, are un-covered and pollinated with the collected fresh male flowers of the male parent, starting as soon as the male flower sheds pollen. The pollinated female flowers are again covered after pollination to prevent bees and any other insects visit. The pollinated female flowers are also marked. The marked flowers are harvested. In some cases, the male pollen used for fertilization has been previously collected and stored.

### Bee-Pollination Method

Using the bee-pollination method, the parent plants are usually planted within close proximity. More female plants may be planted to allow for a greater production of seed. Insects are placed in the field or greenhouses for transfer of pollen from the male parent to the female flowers of the female parent.

### Mass Selection

In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

### Synthetics

A synthetic variety is produced by crossing inter se a number of genotypes selected for good combining ability in all possible hybrid combinations, with subsequent maintenance of the variety by open pollination. Parents are selected on general combining ability, sometimes by test crosses or topcrosses, more generally by polycrosses. Parental seed lines may be deliberately inbred (e.g., by selfing or sib crossing). However, even if the parents are not deliberately inbred, selection within lines during line maintenance will ensure that some inbreeding occurs. Clonal parents will, of course, remain unchanged and highly heterozygous.

### Hybrids

A hybrid is an individual plant resulting from a cross between parents of differing genotypes. Commercial hybrids are now used extensively in many crops, including corn (maize), sorghum, sugar beet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

### Bulk Segregation Analysis (BSA)

BSA, a.k.a. bulked segregation analysis, or bulk segregant analysis, is a method described by Michelmore et al. (Michelmore et al., 1991, Identification of markers linked to disease-resistance genes by bulked segregant analysis: a rapid method to detect markers in specific genomic regions by using segregating populations. Proceedings of the National Academy of Sciences, USA, 99:9828-9832) and Quarrie et al. (Quarrie et al., Bulk segregant analysis with molecular markers and its use for improving drought resistance in maize, 1999, Journal of Experimental Botany, 50(337):1299-1306).

For BSA of a trait of interest, parental lines with certain different phenotypes are chosen and crossed to generate F₂, doubled haploid or recombinant inbred populations with QTL analysis. The population is then phenotyped to identify individual plants or lines having high or low expression of the trait. Two DNA bulks are prepared, one from the individuals having one phenotype (e.g., resistant to pathogen), and the other from the individuals having reversed phenotype (e.g., susceptible to pathogen), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping.

### Gene Pyramiding

The method to combine into a single genotype a series of target genes identified in different parents is usually referred as gene pyramiding. The first part of a gene pyramiding breeding is called a pedigree and is aimed at cumulating one copy of all target genes in a single genotype (called root genotype). The second part is called the fixation steps and is aimed at fixing the target genes into a homozygous state, that is, to derive the ideal genotype (ideotype) from the root genotype. Gene pyramiding can be combined with marker assisted selection (MAS, see Hospital et al., 1992, 1997a, and 1997b, and Moreau et al, 1998) or marker based recurrent selection (MBRS, see Hospital et al., 2000).

Examples of Additional Desired Traits that May be Stacked with the Pathogen Resistance or Tolerance Traits Disclosed Herein

In some cases, multiple FIT1 alleles may be combined in a single plant to increase pathogen resistance. In some cases, one or more FIT1 alleles are combined with additional desired traits. These traits may be introduced to a plant through conventional breeding methods, stacked on one or more DNA constructs, and/or generated through targeted mutagenesis. Examples of additional desired traits include, but are not limited to, male sterility, herbicide resistance, resistance for bacterial, fungal, or viral disease, insect resistance, male fertility, enhanced nutritional quality, industrial usage, yield stability, and yield enhancement. Several of these traits are described in, for example, U.S. Pat. Nos. 5,959,185, 5,973,234, and 5,977,445.

Examples of Plant Species that May be Transformed or Modified, or Serve as a Source of Functional FIT1

The methods disclosed herein may be applied to a wide range of plants. Non-limiting examples of plants which may be transformed or modified using the methods and sequences disclosed herein include, but are not limited to, corn (Zea mays), Brassica spp. (e.g., Brassica napus, Brassica rapa, Brassica juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), broad beans (Viciafaba), tobacco (Nicotiana tabacum), potato (Solanum tuberosum), peanuts (Arachis hypogaea), cotton (Gossypium barbadense, Gossypium hirsutum), quince (Cydonia), sweet potato (Ipomoea batatas), cassava (Manihot esculenta), coffee (Coffea spp.), coconut (Cocos nucifera), pineapple (Ananas comosus), citrus trees (Citrus spp.), apple (Malus spp.), medlar (Mespilus), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), pear (Pyrus), cashew (Anacardium occidentale), macadamia (Macadamia integrifolia), almond (Prunus amygdalus), sugar beets (Beta vulgaris), sugarcane (Saccharum spp.), oats (for example Avena sativa), barley (for example Hordeum vulgare), vegetables and herbs (for example onion, leek, garlic peppermint), ornamentals (for example, Chrysanthemum, Fuchsia spp., Pelargonium, Rosa spp. Primula vulgaris), red cedar (Juniperus virginiana), and conifers (for example juniper (Juniperus communis)).

Examples of plants in the subfamily Papilionoideae include, but are not limited to, Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

Examples of legumes include, but are not limited to, the genus Phaseolus (e.g., French bean, dwarf bean, climbing bean (Phaseolus vulgaris), lima bean (Phaseolus lunatus), Tepary bean (Phaseolus acutifolius), runner bean (Phaseolus coccineus)); the genus Glycine (e.g., Glycine soja, soybeans (Glycine max (L.)); pea (Pisum) (e.g., shelling peas (sometime called smooth or round seeded peas; Pisum sativum); marrowfat pea (Pisum sativum), sugar pea (Pisum sativum), also called snow pea, edible-podded pea or mangetout, (Pisum granda)); peanut (Arachis hypogaea), clover (Trifolium spp.), medick (Medicago), kudzu vine (Pueraria lobata), common lucerne, alfalfa (Medicago sativa), chickpea (Cicer), lentils (Lens culinaris), lupins (Lupinus); vetches (Vicia), field bean, broad bean (Vicia faba), vetchling (Lathyrus) (e.g., chickling pea (Lathyrus sativus), heath pea (Lathyrus tuberosus)); genus Vigna (e.g., moth bean (Vigna aconitifolia), adzuki bean (Vigna angu laris), urd bean (Vigna mungo), mung bean (Vigna radiata), bambara groundnut (Vigna subterrane), rice bean (Vigna umbellata), Vigna vexillata, Vigna unguiculata (also known as asparagus bean, cowpea)); pigeon pea (Cajanus cajan), the genus Macrotyloma (e.g., geocarpa groundnut (Macro tyloma geocarpum), horse bean (Macrotyloma uniflorum; goa bean (Psophocarpus tetragonolobus), African yam bean (Sphenostylis stenocarpa), Egyptian black bean, lablab bean (Lablab purpureus), yam bean (Pachyrhizus erosus), guar bean (Cyamopsis tetragonolobus); and/or the genus Canavalia (e.g., jack bean (Canavalia ensiformis)), sword bean (Canavalia gladiata).

### EXAMPLES

The following examples are provided to illustrate further the various applications and are not intended to limit the disclosure beyond the limitations set forth in the appended claims.

### Example 1: FIT1 Homologs

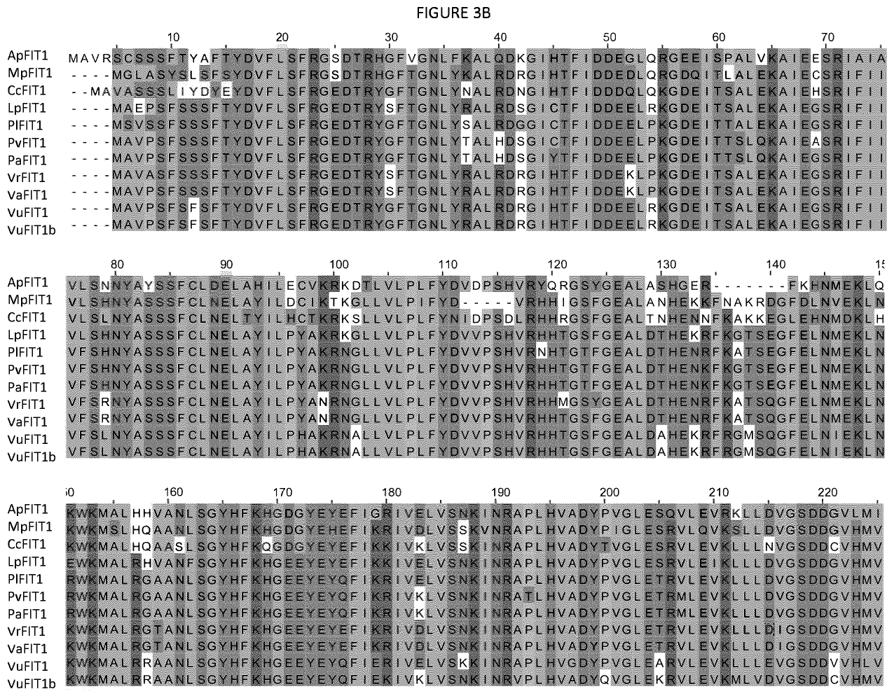

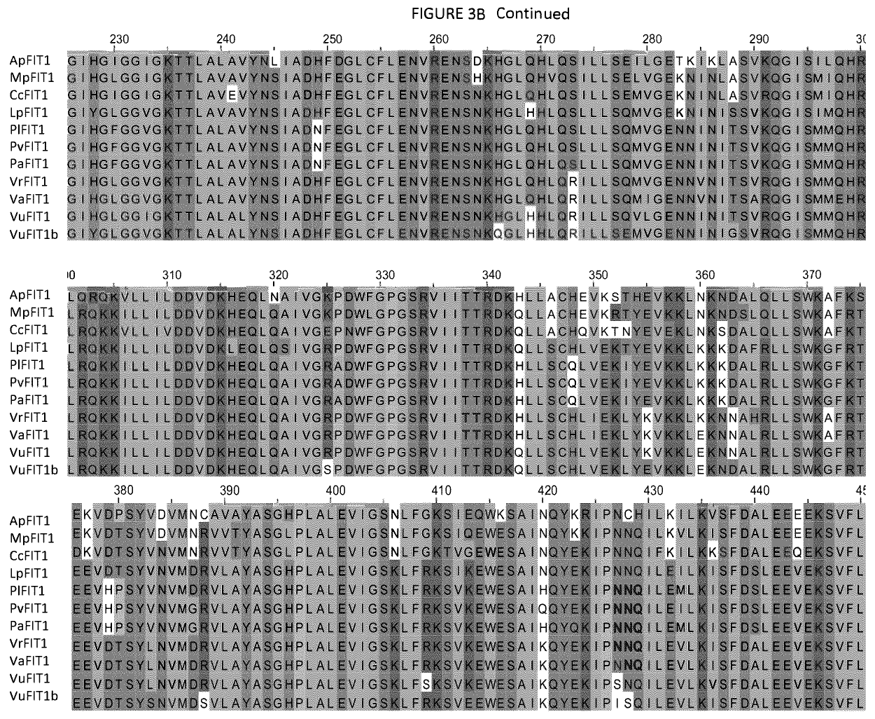

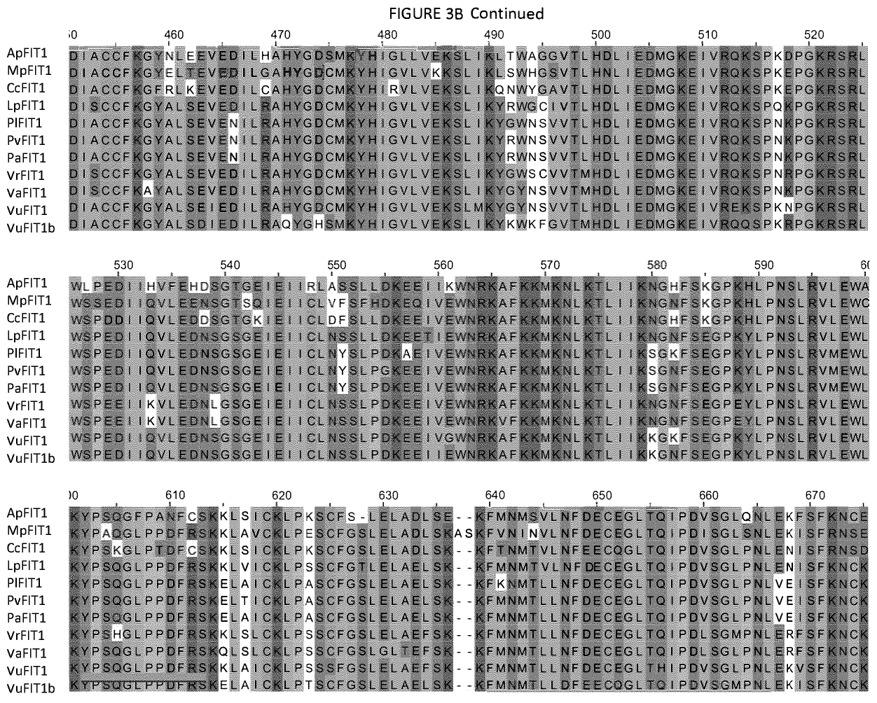

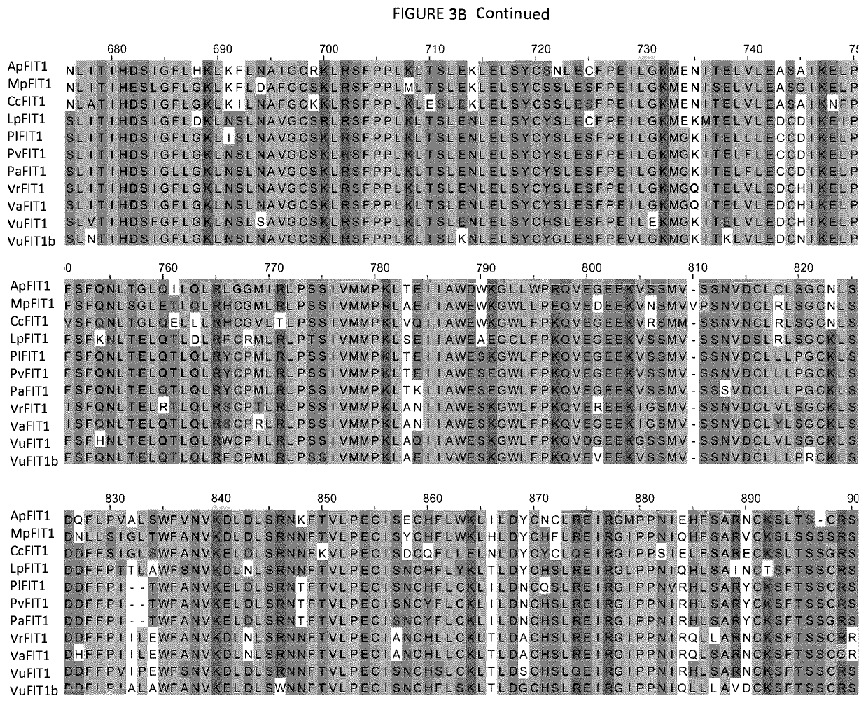

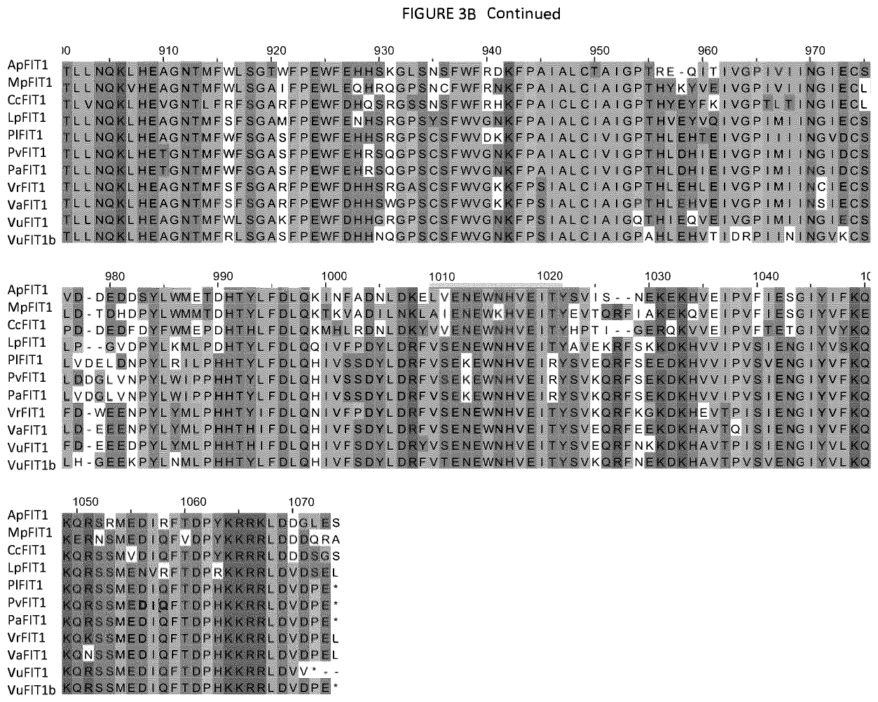

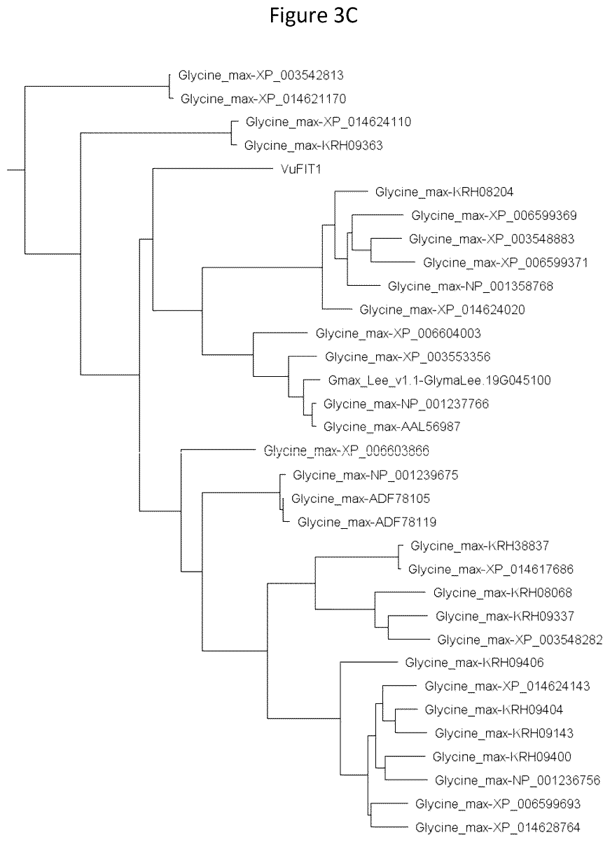

As will be understood by one skilled in the art, homologs of FIT1 may be found in any number of species by methods described herein and methods well known in the art. Examples of homologs of FIT1 identified are shown in FIGS. 3A-3C. FIG. 3A shows a phylogenetic tree of homologs of Vigna unguiculata FIT1 identified by performing a BLAST® search and constructing a protein alignment and phylogenetic tree of the resulting sequences. This figure shows putative FIT1 orthologs in Vigna radiata, Vigna angularis, Phaseolus acutifolius, Phaseolus lunatus, Phaseolus vulgaris, Lablab purpureus, Mucuna pruriens, Cajanus cajan, and Abrus precatorius. The phylogenetic tree was rooted using paralogs of FIT1 that do not function in AvrFIT1 perception.

FIG. 3B shows a protein alignment of the amino acid sequences listed in FIG. 3A, specifically of the Vigna unguiculata allele of FIT1 (VuFIT1) (SEQ ID NO: 2), the Vigna unguiculata close paralog of FIT1 (VuFIT1b) (SEQ ID NO: 22), Vigna angularis allele of FIT1 (VaFIT1) (SEQ ID NO: 12), the Vigna radiata allele of FIT1 (VrFIT1) (SEQ ID NO: 4), the Phaseolus acutifolius allele of FIT1 (PaFIT1) (SEQ ID NO: 10), the Phaseolus vulgaris allele of FIT1 (PvFIT1) (SEQ ID NO: 8), the Phaseolus lunatus allele of FIT1 (PlFIT1) (SEQ ID NO: 6), the Lablab purpureus allele of FIT1 (LpFIT1) (SEQ ID NO: 14), the Cajanus cajun allele of FIT1 (CcFIT1) 1 (SEQ ID NO: 18), the Mucuna pruriens allele of FIT1 (MpFIT1) (SEQ ID NO: 16), and the Abrus precatorius allele of FIT1 (ApFIT1) (SEQ ID NO: 20).

FIG. 3C shows a phylogenetic tree of FIT1 homologs in Glycine max. G. max (soybean) lacks an ortholog of VuFIT1 but contains many homologs of VuFIT1 paralogs, which can be identified by BLAST® search. Protein homologs of VuFIT1 from soybean were obtained from NCBI and Phytozome, curated for completeness and to remove duplicates, and used to generate the phylogenetic tree shown in FIG. 3C. The tree was rooted using an outgroup of distantly related TIR-NLR proteins from non-legumes.

Sequence alignments of FIT1 homologs with functional FIT1 genes (such as VuFIT1) can show what genetic changes could be induced to restore recognition of AvrFIT, and confer resistance or tolerance to a pathogen. Such targeted genetic editing is well known in the art and also described herein.

### Example 2: Transient Expression of FIT1 Alleles in Leaf Tissue

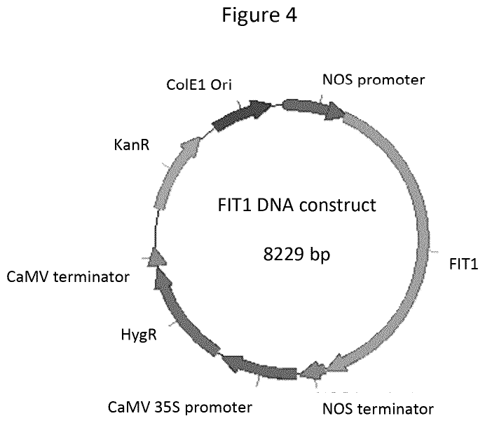

Leaf tissue from a plant lacking an endogenous FIT1 was transformed with constructs containing various FIT1 sequences, AvrFIT1a, AvrFIT1b, and/or Bs3 using standard transformation technology (an example construct comprising VuFIT1 is shown in FIG. 4). Suspensions containing the desired expression constructs were infiltrated into the leaf tissue (OD₆₀₀=0.4 total) using needleless syringe and imaged four days post infiltration. The site of each infiltration is visible by a small punch through the leaf.

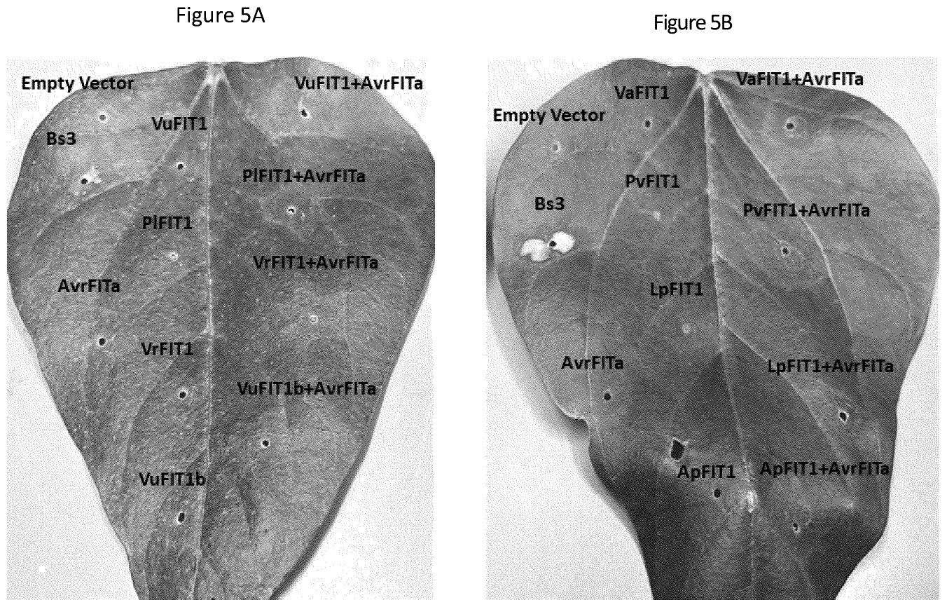

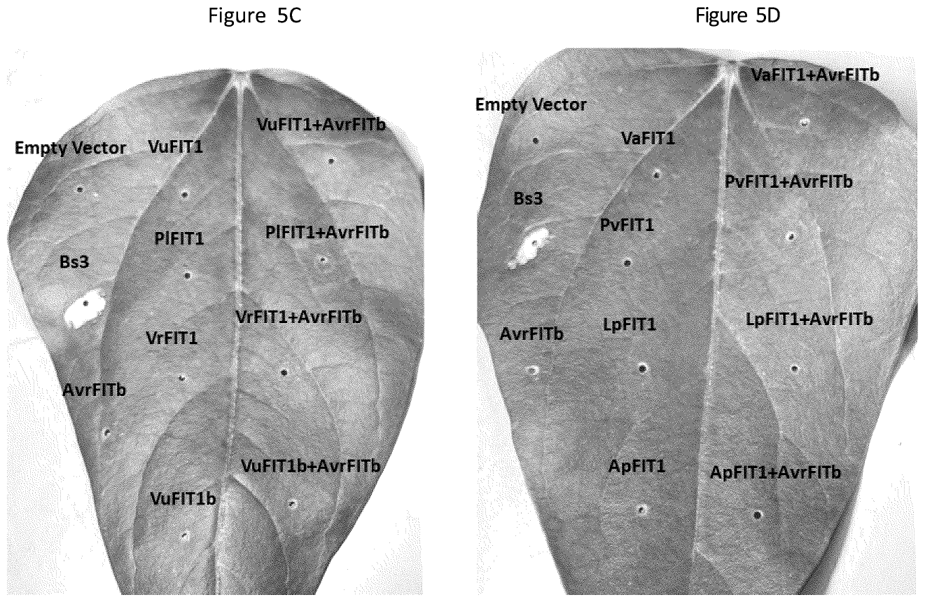

As shown in FIGS. 5A and 5B, co-expression of AvrFIT1a (SEQ ID NO: 23) with either Vigna unguiculata (VuFIT1) (SEQ ID NO: 27), Phaseolus lunatus (PlFIT1) (SEQ ID NO: 28), Vigna radiata (VrFIT1) (SEQ ID NO: 29), Vigna angularis (VaFIT1) (SEQ ID NO: 31), Phaseolus vulgaris (PvFIT1) (SEQ ID NO: 32), Lablab purpureus (LpFIT1) (SEQ ID NO: 33), or Abrus precatoris (ApFIT1) (SEQ ID NO: 34) resulted in a strong cell death response, indicative of immune activation, but no response was observed when the proteins were expressed individually. No immune response was observed when the non-functional paralog VuFIT1b (SEQ ID NO: 30) was expressed individually or with AvrFIT1a.

As shown in FIGS. 5C and 5D, co-expression of AvrFIT1b (SEQ ID NO: 25) with either Phaseolus lunatus (PlFIT1), Vigna radiata (VrFIT1), Vigna angularis (VaFIT1), Phaseolus vulgaris (PvFIT1), or Lablab purpureus (LpFIT1) resulted in a cell death response, indicative of immune activation, but no response was observed when the proteins were expressed individually.

Expression of the executor Bs3, a positive control for cell death response, triggers a similar response. This demonstrates that FIT1 mediates the perception of AvrFIT1, and thus can confer resistance or tolerance to pathogens that secrete the effector protein AvrFIT1.

### Example 3: Stable Expression of Vigna unguiculata FIT1 in Glycine max

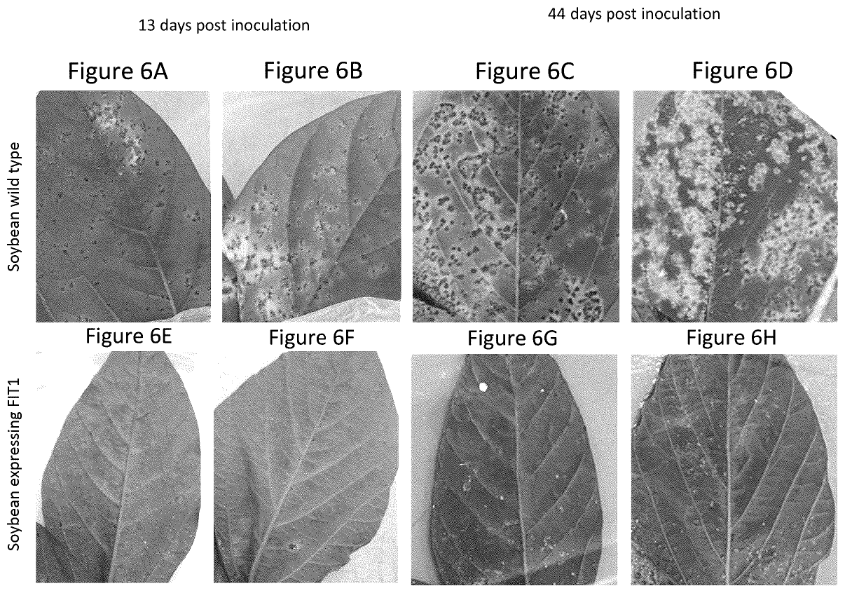

Soybean plants stably expressing VuFIT1 (SEQ ID NO: 1, example construct shown in FIG. 4) were generated and tested for resistance to ASR (Phakopsora pachyrhizi). FIGS. 6A-6D depicts wild type soybean leaves lacking functional FIT1 (top row) and FIGS. 6E-6H show leaves from soybean plants expressing VuFIT1 (bottom row). Plants were inoculated with Phakopsora pachyrhizi and the leaves were photographed at 13-days (FIGS. 6A-6B and 6E-6F) and 44-days (FIGS. 6C-6D and 6G-6H) post inoculation. The wild type soybean leaves showed susceptibility to Phakopsora as seen by the development of large lesions and many fungal spores. However, soybean expressing VuFIT1 showed strong resistance to the pathogen (FIGS. 6E-6H).

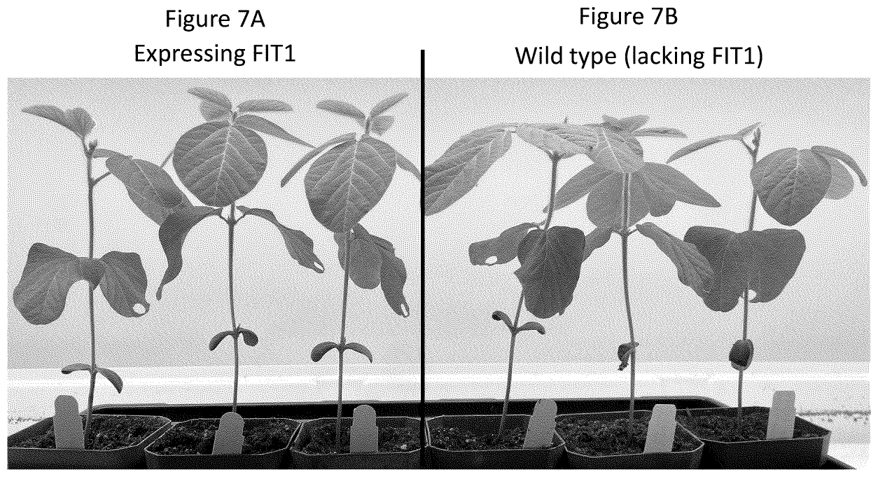

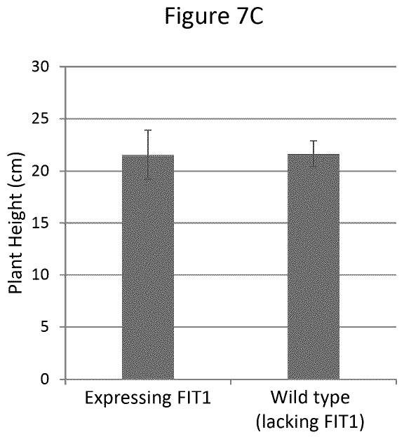

Transgenic expression of VuFIT1 in soybean did not affect plant growth or morphology. Photographs of wild type soybean plants (FIG. 7A) and transgenic soybean plants expressing VuFIT1 (FIG. 7B) had no obvious growth abnormalities. The height of the plants was measured at 24 days after planting and no significant difference was observed between the wild type plants and the plants containing FIT1 (FIG. 7C). The error bars indicate the standard deviation of the plant height from the individual plants (n>8).

### Example 4: Introducing FIT1 Homologs into Other Plant Species

The FIT1 sequences isolated and described herein can be introduced into other plant species to create a plant having resistance or tolerance to a pathogen.

For example, a sequence encoding any one of the proteins of SEQ ID NOs: 2, 4, 6, 8, 10, 12, 14, 16, 18, 20 and functional homologs thereof, or sequences of SEQ ID NOs: 1, 3, 5, 7, 9, 11, 13, 15, 17, 19 complements thereof, fragments thereof, and sequences at least 70% identical thereto can be introduced into a plant to confer resistance or tolerance to a pathogen. For example, as described above, Glycine max, which does not possess a functional FIT1 gene and is susceptible to ASR, may be transformed with a transgene comprising SEQ ID NO: 1, or a nucleotide sequence encoding the amino acid sequence of SEQ ID NO: 2, to confer resistance to plant pathogens, such as Phakopsora pachyrhizi, that secrete the effector protein AvrFIT1, and cause diseases like ASR. Based on the transient assays described herein (FIG. 5A-5D), resistance could also be achieved with a transgene comprising any one of SEQ ID NOs: 3, 5, 7, 9, 11, 13, 15, 17, and/or 19, or a sequence encoding any one of the proteins of SEQ ID NOs: 2, 4, 6, 8, 10, 12, 14, 16, 18, and/or 20.

Additional plant species susceptible to ASR or pathogens secreting AvrFIT1-like effector proteins, such as Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp. could also be transformed with any of the FIT1 sequences disclosed herein to confer resistance to a pathogen.

### Example 5: Methods of Identifying Pathogen Resistant Genes

FIT1 orthologs are likely present in additional species and genera within the Fabaceae family. FIT1 orthologs may identified by any number of means known in the art. This includes sequencing the genome or transcriptome of a plant species, identifying FIT1 homologs using a BLAST search, identifying putative FIT1 orthologs by constructing a phylogenetic tree of the homologous proteins, and then testing the identified putative FIT1 genes for AvrFIT1 recognition activity using a transient assay such as that shown in FIG. 5A-5D. Alternatively, synthetic alleles of FIT1 may be designed by combining fragments of naturally occurring FIT1 alleles or by introducing amino acid substitutions at positions shown to be variable in an alignment of functional FIT1 proteins and similarly tested for functionality by transient assay. Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

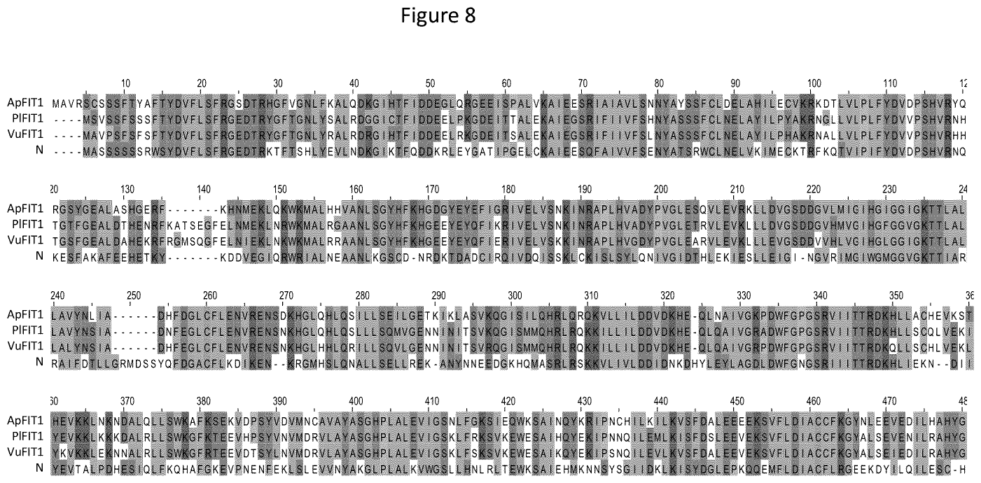

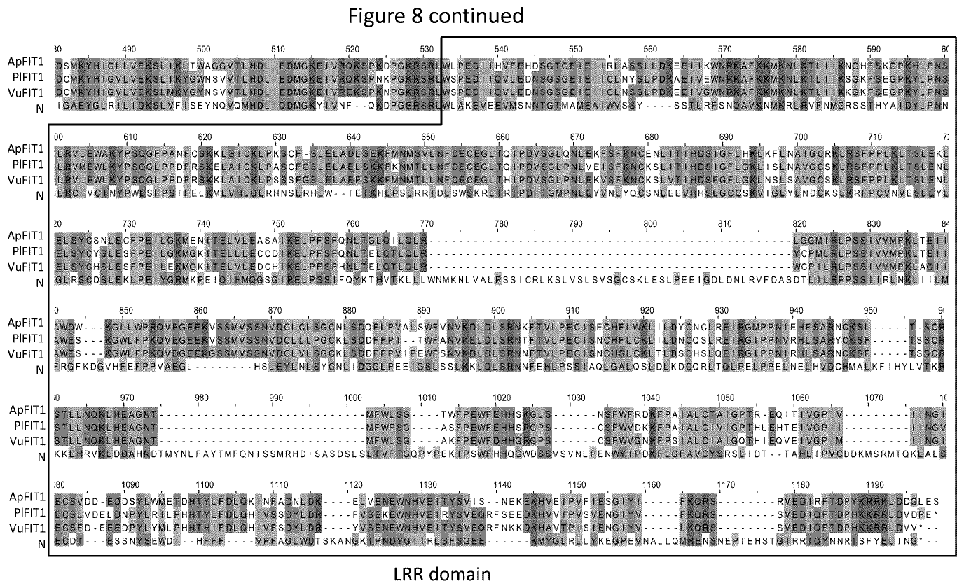

While previous reports have listed some FIT1 homologs as predicted TMV resistance proteins, this is unlikely. FIG. 8 depicts an alignment between the FIT1 alleles from Vigna unguiculata (VuFIT1), Phaseolus lunatus (PlFIT1), Abrus precatorius (ApFIT1), and the N gene, which gives TMV resistance. The LRR domain is poorly conserved between the FIT1 alleles and the N gene. Therefore, FIT1 is not expected to have the same activity as the N gene (which recognizes the P50 protein and confers resistance to Tobacco Mosaic Virus). This prediction can be confirmed by transient expression of the proteins, which demonstrates that the N gene can recognize P50 but not AvrFIT1a or AvrFITb and that FIT1 is not able to recognize P50.

The ability of other potential FIT1 homologs or synthetic genes to function for AvrFIT1 perception can be easily and quickly tested using the transient expression assays shown and described herein or similar methods well known in the art. For example, once a FIT1 ortholog is identified in a plant species that is resistant to ASR, the gene can be cloned and tested for effector protein recognition using transient expression assays and the AvrFIT1a (SEQ ID NO: 23) and AvrFIT1b sequences (SEQ ID NO: 25) described herein (see for example FIGS. 5A-D). Examples of leaf tissue suitable for use in a FIT1-AvrFIT1 assay include, but are not limited to, species or accessions of Nicotiana, Solanum, Physalis, Capsicum, Lactuca, Alysicarpus, Astragalus, Baptisia, Cajanus, Calopogonium, Caragana, Centrosema, Cologania, Crotalaria, Desmodium, Genista, Glycine, Glycyrrhiza, Indigofera, Kummerowia, Lablab, Lathyrus, Lespedeza, Lotus, Lupinus, Macroptilium, Macrotyloma, Medicago, Neonotonia, Pachyrhizus, Pisum, Phaseolus, Pseudovigna, Psoralea, Robinia, Senna, Sesbania, Strophostyles, Tephrosia, Teramnus, Trifolium, Vicia, Vigna, and Voandzeia that lack a functional native FIT1 gene. Similarly, resistance genes for other pathogens may be identified using this same method, wherein a potential gene is identified, cloned, and tested in transient assays with pathogen effector proteins.

### BRIEF DESCRIPTION OF THE SEQUENCE LISTINGS

SEQ ID NO: 1 shows the nucleic acid sequence of Vigna unguiculata FIT1.

SEQ ID NO: 2 shows the corresponding amino acid sequence of SEQ ID NO: 1.

SEQ ID NO: 3 shows the nucleic acid sequence of Vigna radiata FIT1.

SEQ ID NO: 4 shows the corresponding amino acid sequence of SEQ ID NO: 3.

SEQ ID NO: 5 shows the nucleic acid sequence of Phaseolus lunatus FIT1.

SEQ ID NO: 6 shows the corresponding amino acid sequence of SEQ ID NO: 5.

SEQ ID NO: 7 shows the nucleic acid sequence of Phaseolus vulgaris FIT1.

SEQ ID NO: 8 shows the corresponding amino acid sequence of SEQ ID NO: 7.

SEQ ID NO: 9 shows the nucleic acid sequence of Phaseolus acutifolius FIT1.

SEQ ID NO: 10 shows the corresponding amino acid sequence of SEQ ID NO: 9.

SEQ ID NO: 11 shows the nucleic acid sequence of Vigna angularis FIT1.

SEQ ID NO: 12 shows the corresponding amino acid sequence of SEQ ID NO: 11.

SEQ ID NO: 13 shows the nucleic acid sequence of Lablab purpureus FIT1.

SEQ ID NO: 14 shows the corresponding amino acid sequence of SEQ ID NO: 13.

SEQ ID NO: 15 shows the nucleic acid sequence of Mucuna pruriens FIT1.

SEQ ID NO: 16 shows the corresponding amino acid sequence of SEQ ID NO: 15.

SEQ ID NO: 17 shows the nucleic acid sequence of Cajanus cajun FIT1.

SEQ ID NO: 18 shows the corresponding amino acid sequence of SEQ ID NO: 17.

SEQ ID NO: 19 shows the nucleic acid sequence of Abrus precatorius FIT1.

SEQ ID NO: 20 shows the corresponding amino acid sequence of SEQ ID NO: 19.

SEQ ID NO: 21 shows the nucleic acid sequence of Vigna unguiculata FIT1b.

SEQ ID NO: 22 shows the corresponding amino acid sequence of SEQ ID NO: 21.

SEQ ID NO: 23 shows the nucleic acid sequence of Phakopsora pachyrhizi ALL40704.1 (AvrFIT1a).

SEQ ID NO: 24 shows the corresponding amino acid sequence of SEQ ID NO: 23.

SEQ ID NO: 25 shows the nucleic acid sequence of Phakopsora pachyrhizi ALL41167.1 (AvrFIT1b).

SEQ ID NO: 26 shows the corresponding amino acid sequence of SEQ ID NO: 25.

SEQ ID NO: 27 shows the nucleic acid sequence of the transient expression vector with Vigna unguiculata FIT1.

SEQ ID NO: 28 shows the nucleic acid sequence of the transient expression vector with Phaseolus lunatus FIT1.

SEQ ID NO: 29 shows the nucleic acid sequence of the transient expression vector with Vigna radiata FIT1.

SEQ ID NO: 30 shows the nucleic acid sequence of the transient expression vector with Vigna unguiculata FIT1b.

SEQ ID NO: 31 shows the nucleic acid sequence of the transient expression vector with Vigna angularis FIT1.

SEQ ID NO: 32 shows the nucleic acid sequence of the transient expression vector with Phaseolus vulgaris FIT1.

SEQ ID NO: 33 shows the nucleic acid sequence of the transient expression vector with Lablab purpureus FIT1.

SEQ ID NO: 34 shows the nucleic acid sequence of the transient expression vector with Abrus precatorius FIT1.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### Numbered Embodiments

1. An isolated, recombinant, or synthetic polynucleotide comprising a nucleic acid sequence encoding a functional FIT1 protein homologous to SEQ ID NO: 2.
2. The isolated, recombinant, or synthetic polynucleotide of embodiment 1, wherein the polynucleotide encodes a protein having at least 70% identity to SEQ ID NO: 2.
3. The isolated, recombinant, or synthetic polynucleotide of embodiment 2, wherein the polynucleotide encodes a FIT1 protein from Abrus precatorius.
4. The isolated, recombinant, or synthetic polynucleotide of embodiment 3, wherein the polynucleotide comprises SEQ ID NO: 19, a polynucleotide encoding SEQ ID NO: 20, complements thereof, or fragments thereof.
5. The isolated, recombinant, or synthetic polynucleotide of embodiment 2, wherein the polynucleotide encodes a FIT1 protein from Cajanus cajan.
6. The isolated, recombinant, or synthetic polynucleotide of embodiment 5, wherein the polynucleotide comprises SEQ ID NO: 17, or a polynucleotide encoding SEQ ID NO: 18.
7. The isolated, recombinant, or synthetic polynucleotide of embodiment 1, wherein the polynucleotide encodes a protein having at least 75% identity to SEQ ID NO: 2.
8. The isolated, recombinant, or synthetic polynucleotide of embodiment 7, wherein the polynucleotide encodes a FIT1 protein from Mucuna pruriens.
9. The isolated, recombinant, or synthetic polynucleotide of embodiment 8, wherein the polynucleotide comprises SEQ ID NO: 15, a polynucleotide encoding SEQ ID NO: 16, complements thereof, or fragments thereof.
10. The isolated, recombinant, or synthetic polynucleotide of embodiment 1, wherein the polynucleotide encodes a protein having at least 80% identity to SEQ ID NO: 2.
11. The isolated, recombinant, or synthetic polynucleotide of embodiment 1, wherein the polynucleotide encodes a protein having at least 85% identity to SEQ ID NO: 2.
12. The isolated, recombinant, or synthetic polynucleotide of embodiment 11, wherein the polynucleotide encodes a FIT1 protein from Lablab purpureus.
13. The isolated, recombinant, or synthetic polynucleotide of embodiment 12, wherein the polynucleotide comprises SEQ ID NO: 13, a polynucleotide encoding SEQ ID NO: 14, complements thereof, or fragments thereof.
14. The isolated, recombinant, or synthetic polynucleotide of embodiment 11, wherein the polynucleotide encodes a FIT1 protein from Phaseolus lunatus.
15. The isolated, recombinant, or synthetic polynucleotide of embodiment 14, wherein the polynucleotide comprises SEQ ID NO: 5, a polynucleotide encoding SEQ ID NO: 6, complements thereof, or fragments thereof.
16. The isolated, recombinant, or synthetic polynucleotide of embodiment 11, wherein the polynucleotide encodes a FIT1 protein from Phaseolus vulgaris.
17. The isolated, recombinant, or synthetic polynucleotide of embodiment 16, wherein the polynucleotide comprises SEQ ID NO: 7, a polynucleotide encoding SEQ ID NO: 8, complements thereof, or fragments thereof.
18. The isolated, recombinant, or synthetic polynucleotide of embodiment 11, wherein the polynucleotide encodes a FIT1 protein from Phaseolus acutifolius.
19. The isolated, recombinant, or synthetic polynucleotide of embodiment 18, wherein the polynucleotide comprises SEQ ID NO: 9, a polynucleotide encoding SEQ ID NO: 10, complements thereof, or fragments thereof.
20. The isolated, recombinant, or synthetic polynucleotide of embodiment 11, wherein the polynucleotide encodes a FIT1 protein from Vigna radiata.
21. The isolated, recombinant, or synthetic polynucleotide of embodiment 20, wherein the polynucleotide comprises SEQ ID NO: 3, a polynucleotide encoding SEQ ID NO: 4, complements thereof, or fragments thereof.
22. The isolated, recombinant, or synthetic polynucleotide of embodiment 1, wherein the polynucleotide encodes a protein having at least 90% identity to SEQ ID NO: 2.
23. The isolated, recombinant, or synthetic polynucleotide of embodiment 22, wherein the polynucleotide encodes a FIT1 protein from Vigna angularis.
24. The isolated, recombinant, or synthetic polynucleotide of embodiment 23, wherein the polynucleotide comprises SEQ ID NO: 11, a polynucleotide encoding SEQ ID NO: 12, complements thereof, or fragments thereof.
25. The isolated, recombinant, or synthetic polynucleotide of embodiment 1, wherein the polynucleotide encodes a protein having at least 95% identity to SEQ ID NO: 2.
26. The isolated, recombinant, or synthetic polynucleotide of embodiment 1, wherein the polynucleotide encodes a protein having at least 96% identity to SEQ ID NO: 2.
27. The isolated, recombinant, or synthetic polynucleotide of embodiment 1, wherein the polynucleotide encodes a protein having at least 97% identity to SEQ ID NO: 2.
28. The isolated, recombinant, or synthetic polynucleotide of embodiment 1, wherein the polynucleotide encodes a protein having at least 98% identity to SEQ ID NO: 2.
29. The isolated, recombinant, or synthetic polynucleotide of embodiment 1, wherein the polynucleotide encodes a protein having at least 99% identity to SEQ ID NO: 2.
30. An isolated, recombinant, or synthetic polynucleotide comprising a nucleic acid sequence encoding a FIT1 protein, wherein the protein is selected from the group consisting of: SEQ ID NOs: 2, 4, 6, 8, 10, 12, 14, 16, 18, 20 and functional homologs thereof.
31. The isolated, recombinant, or synthetic polynucleotide of embodiment 30, wherein the polynucleotide comprises a nucleic acid sequence selected from the group consisting of SEQ ID NOs: 1, 3, 5, 7, 9, 11, 13, 15, 17, 19 complements thereof, fragments thereof, and sequences at least 70% identical thereto.
32. A genetic construct comprising at least one of the nucleic acid sequences of any one of embodiments 1-31.
33. A plant, plant part, or plant cell transformed with at least one of the nucleic acid sequences of any one of embodiments 1-31 or the genetic construct of embodiment 32, wherein said plant, plant part or plant cell is resistant or tolerant to a pathogen.
34. The plant, plant part, or plant cell of embodiment 33, wherein the pathogen is a fungus from the order Cantharellales or Pucciniales.
35. The plant, plant part, or plant cell of embodiment 33 or 34, wherein the fungal pathogen is Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp. or Hemileia vastatrix.
36. The plant, plant part, or plant cell of any one of embodiments 33-35, wherein the plant, plant part, or plant cell is in the subfamily Papilionoideae.
37. The plant, plant part, or plant cell of any one of embodiments 33-36, wherein the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.
38. The plant, plant part, or plant cell of any one of embodiments 33-37, wherein the plant, plant part, or plant cell is Glycine max, and wherein the plant, plant part, or plant cell is resistant to Asian Soybean Rust caused by Phakopsora pachyrhizi.
39. The plant, plant part, or plant cell of embodiment 38, wherein the resistance to Asian Soybean Rust is conferred by a transgene comprising SEQ ID NO: 1, or a nucleotide sequence encoding the amino acid sequence of SEQ ID NO: 2.
40. The plant, plant part, or plant cell of embodiment 38, wherein the resistance to Asian Soybean Rust is conferred by a transgene comprising SEQ ID NO: 3, or a nucleotide sequence encoding the amino acid sequence of SEQ ID NO: 4.
41. The plant, plant part, or plant cell of embodiment 38, wherein the resistance to Asian Soybean Rust is conferred by a transgene comprising SEQ ID NO: 5, or a nucleotide sequence encoding the amino acid sequence of SEQ ID NO: 6.
42. The plant, plant part, or plant cell of embodiment 38, wherein the resistance to Asian Soybean Rust is conferred by a transgene comprising SEQ ID NO: 7, or a nucleotide sequence encoding the amino acid sequence of SEQ ID NO: 8.
43. The plant, plant part, or plant cell of embodiment 38, wherein the resistance to Asian Soybean Rust is conferred by a transgene comprising SEQ ID NO: 9, or a nucleotide sequence encoding the amino acid sequence of SEQ ID NO: 10.
44. The plant, plant part, or plant cell of embodiment 38, wherein the resistance to Asian Soybean Rust is conferred by a transgene comprising SEQ ID NO: 11, or a nucleotide sequence encoding the amino acid sequence of SEQ ID NO: 12.
45. The plant, plant part, or plant cell of embodiment 38, wherein the resistance to Asian Soybean Rust is conferred by a transgene comprising SEQ ID NO: 13, or a nucleotide sequence encoding the amino acid sequence of SEQ ID NO: 14.
46. The plant, plant part, or plant cell of embodiment 38, wherein the resistance to Asian Soybean Rust is conferred by a transgene comprising SEQ ID NO: 15, or a nucleotide sequence encoding the amino acid sequence of SEQ ID NO: 16.
47. The plant, plant part, or plant cell of embodiment 38, wherein the resistance to Asian Soybean Rust is conferred by a transgene comprising SEQ ID NO: 17, or a nucleotide sequence encoding the amino acid sequence of SEQ ID NO: 18.
48. The plant, plant part, or plant cell of embodiment 38, wherein the resistance to Asian Soybean Rust is conferred by a transgene comprising SEQ ID NO: 19, or a nucleotide sequence encoding the amino acid sequence of SEQ ID NO: 20.
49. A method of producing a plant, plant part, or plant cell having resistance or tolerance to a pathogen, wherein the method comprises:
   transforming a plant, plant part, or plant cell with a nucleotide sequence encoding a Toll-like Interleukin-1 Receptor (TIR) Nucleotide binding, Leucine-rich Repeat (NLR) immune receptor protein, wherein said immune receptor protein mediates the perception of pathogen effector protein AvrFIT1 or homologs thereof, and wherein expression of the immune receptor protein prevents the pathogen from colonizing the plant, or prevents the pathogen from affecting plant growth or yield.
50. The method of embodiment 49, wherein the pathogen effector protein comprises SEQ ID NO: 24, SEQ ID: 26, or sequences at least 90% identical thereto.
51. The method of embodiment 49 or 50, wherein the nucleotide sequence encoding the immune receptor protein has been codon optimized.
52. The method of any one of embodiments 49-51, wherein the immune receptor protein is selected from the group consisting of:
   an isolated, recombinant, or synthetic polynucleotide comprising a nucleic acid sequence encoding a FIT1 protein, wherein the protein is selected from the group consisting of: SEQ ID NOs: 2, 4, 6, 8, 10, 12, 14, 16, 18, 20 and functional homologs thereof, or
   an isolated, recombinant, or synthetic polynucleotide encoding a FIT1 protein, wherein the nucleic acid sequence is selected from the group consisting of SEQ ID NOs: 1, 3, 5, 7, 9, 11, 13, 15, 17, 19 complements thereof, fragments thereof, and sequences at least 70% identical thereto.
53. The method of any one of embodiments 49-52, wherein the plant, plant part, or plant cell is transformed with one or more additional desired traits.
54. The method of embodiment 49, wherein the one or more additional desired traits are stacked together with the immune receptor protein on the same DNA construct.
55. The method of any one of embodiments 49-23, further comprising introgressing one or more additional desired traits.
56. The method of any one of embodiments 49-55, wherein the one or more additional desired traits are resistance traits to a disease, pest, or abiotic stress.
57. A plant, plant part, or plant cell produced by the method of any one of embodiments 49-56, wherein the plant, plant part, or plant cell is resistant to a pathogen.
58. A plant, plant part, or plant cell produced by the method of any one of embodiments 49-56, wherein the plant, plant part, or plant cell is tolerant to a pathogen.
59. The plant, plant part, or plant cell of embodiment 57 or 58, wherein the immune receptor protein is transiently expressed.
60. The plant, plant part, or plant cell of embodiment 57 or 58, wherein the immune receptor protein is stably expressed.
61. The plant, plant part, or plant cell of any one of embodiments 49-60, wherein the plant, plant part, or plant cell is in the subfamily Papilionoideae.
62. The plant, plant part, or plant cell of embodiment 61, wherein the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.
63. The plant, plant part, or plant cell of any one of embodiments 57-62, wherein the plant is Glycine max, wherein the plant, plant part, or plant cell is resistant to Asian Soybean Rust caused by Phakopsora pachyrhizi.
64. A method of genetically engineering a pathogen resistance or tolerance trait in a plant, plant part, or plant cell, comprising:
   providing a plant species that is susceptible to a pathogen;
   identifying within the genome of the plant species a homolog of FIT1, wherein said homolog does not mediate AvrFIT1 recognition; and
   genetically modifying a plant, plant part, or plant cell from the susceptible plant species with targeted gene editing, wherein said targeted gene editing is directed at the FIT1 homolog, and wherein said targeted gene editing enables the FIT1 homolog to recognize AvrFIT1 and confers resistance or tolerance to a pathogen.
65. The method of embodiment 64, wherein the targeted gene editing uses an engineered or natural nuclease selected from the group consisting of homing endonucleases/meganucleases (EMNs), zinc finger nucleases (ZFNs), and transcription activator-like effector nucleases (TALENs).
66. The method of embodiment 64 or 65, wherein targeted gene editing uses a clustered regularly interspaced short palindromic repeats (CRISPR)-Cas nuclease.
67. The method of embodiment 66, wherein the nuclease is selected from the group consisting of Cas9, Cas12, Cas13, CasX, and CasY.
68. The method of any one of embodiments 64-67, further comprising breeding with, or asexually propagating the plant.
69. A genetically modified plant, plant part, or plant cell produced by the method of any one of embodiments 64-68, wherein said plant, plant part, or plant cell exhibits resistance or tolerance to a pathogen.
70. The genetically modified plant, plant part, or plant cell of embodiment 69, wherein the pathogen is a fungus from the order Cantharellales or Pucciniales.
71. The genetically modified plant, plant part, or plant cell of embodiment 70, wherein the fungal pathogen is Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp. or Hemileia vastatrix.
72. The genetically modified plant, plant part, or plant cell of any one of embodiments 69-71, wherein the fungal pathogen is Phakopsora pachyrhizi and the plant, plant part, or plant cell is Glycine max.
73. A method for identifying a functional FIT1 gene and/or allele thereof comprising:
   isolating a FIT1 homolog or allele thereof,
   expressing all or a substantial fragment of said FIT1 homolog or allele thereof in combination with a homolog of AvrFIT1 in a plant, plant part, or plant cell; and
   assaying said plant, plant part, or plant cell for an immune response.
74. The method of embodiment 73, wherein the effector protein comprises SEQ ID NO: 24, SEQ ID: 26, or sequences at least 90% identical thereto.
75. The method of embodiment 73 or 74, wherein the FIT1 allele is a synthetic variant.
76. The method of any one of embodiments 73-75, wherein the plant, plant part, or plant cell is a species of Nicotiana, Solanum, Physalis, Capsicum, Lactuca, Alysicarpus, Astragalus, Baptisia, Cajanus, Calopogonium, Caragana, Centrosema, Cologania, Crotalaria, Desmodium, Genista, Glycine, Glycyrrhiza, Indigofera, Kummerowia, Lablab, Lathyrus, Lespedeza, Lotus, Lupinus, Macroptilium, Macrotyloma, Medicago, Neonotonia, Pachyrhizus, Pisum, Phaseolus, Pseudovigna, Psoralea, Robinia, Senna, Sesbania, Strophostyles, Tephrosia, Teramnus, Trifolium, Vicia, Vigna, and Voandzeia that lacks a functional native FIT1 gene.
77. A transgenic plant, plant part, or plant cell having resistance to Asian Soybean Rust, wherein the resistance is conferred by a transgene comprising SEQ ID NO: 1, or a sequence at least 70% identical thereto.
78. A transgenic plant, plant part, or plant cell having resistance to Asian Soybean Rust, wherein the resistance is conferred by a transgene comprising SEQ ID NO: 1, or a sequence at least 80% identical thereto.
79. A transgenic plant, plant part, or plant cell having resistance to Asian Soybean Rust, wherein the resistance is conferred by a transgene comprising SEQ ID NO: 1, or a sequence at least 90% identical thereto.
80. A transgenic plant, plant part, or plant cell having resistance to Asian Soybean Rust, wherein the resistance is conferred by a transgene comprising a nucleotide sequence encoding the amino acid sequence of SEQ ID NO: 2, or an amino acid sequence at least 90% identical thereto.
81. A transgenic plant, plant part, or plant cell having resistance to Asian Soybean Rust, wherein the resistance is conferred by a transgene comprising SEQ ID NO: 3, or a sequence at least 70% identical thereto.
82. A transgenic plant, plant part, or plant cell having resistance to Asian Soybean Rust, wherein the resistance is conferred by a transgene comprising SEQ ID NO: 3, or a sequence at least 80% identical thereto.
83. A transgenic plant, plant part, or plant cell having resistance to Asian Soybean Rust, wherein the resistance is conferred by a transgene comprising SEQ ID NO: 3, or a sequence at least 90% identical thereto.
84. A transgenic plant, plant part, or plant cell having resistance to Asian Soybean Rust, wherein the resistance is conferred by a transgene comprising a nucleotide sequence encoding the amino acid sequence of SEQ ID NO: 4, or an amino acid sequence at least 90% identical thereto.
85. A transgenic plant, plant part, or plant cell having resistance to Asian Soybean Rust, wherein the resistance is conferred by a transgene comprising SEQ ID NO: 5, or a sequence at least 70% identical thereto.
86. A transgenic plant, plant part, or plant cell having resistance to Asian Soybean Rust, wherein the resistance is conferred by a transgene comprising SEQ ID NO: 5, or a sequence at least 80% identical thereto.
87. A transgenic plant, plant part, or plant cell having resistance to Asian Soybean Rust, wherein the resistance is conferred by a transgene comprising SEQ ID NO: 5, or a sequence at least 90% identical thereto.
88. A transgenic plant, plant part, or plant cell having resistance to Asian Soybean Rust, wherein the resistance is conferred by a transgene comprising a nucleotide sequence encoding the amino acid sequence of SEQ ID NO: 6, or an amino acid sequence at least 90% identical thereto.
89. A transgenic plant, plant part, or plant cell having resistance to Asian Soybean Rust, wherein the resistance is conferred by a transgene comprising SEQ ID NO: 7, or a sequence at least 70% identical thereto.
90. A transgenic plant, plant part, or plant cell having resistance to Asian Soybean Rust, wherein the resistance is conferred by a transgene comprising SEQ ID NO: 7, or a sequence at least 80% identical thereto.
91. A transgenic plant, plant part, or plant cell having resistance to Asian Soybean Rust, wherein the resistance is conferred by a transgene comprising SEQ ID NO: 7, or a sequence at least 90% identical thereto.
92. A transgenic plant, plant part, or plant cell having resistance to Asian Soybean Rust, wherein the resistance is conferred by a transgene comprising a nucleotide sequence encoding the amino acid sequence of SEQ ID NO: 8, or an amino acid sequence at least 90% identical thereto.
93. A transgenic plant, plant part, or plant cell having resistance to Asian Soybean Rust, wherein the resistance is conferred by a transgene comprising SEQ ID NO: 9, or a sequence at least 70% identical thereto.
94. A transgenic plant, plant part, or plant cell having resistance to Asian Soybean Rust, wherein the resistance is conferred by a transgene comprising SEQ ID NO: 9, or a sequence at least 80% identical thereto.
95. A transgenic plant, plant part, or plant cell having resistance to Asian Soybean Rust, wherein the resistance is conferred by a transgene comprising SEQ ID NO: 9, or a sequence at least 90% identical thereto.
96. A transgenic plant, plant part, or plant cell having resistance to Asian Soybean Rust, wherein the resistance is conferred by a transgene comprising a nucleotide sequence encoding the amino acid sequence of SEQ ID NO: 10, or an amino acid sequence at least 90% identical thereto.
97. A transgenic plant, plant part, or plant cell having resistance to Asian Soybean Rust, wherein the resistance is conferred by a transgene comprising SEQ ID NO: 11, or a sequence at least 70% identical thereto.
98. A transgenic plant, plant part, or plant cell having resistance to Asian Soybean Rust, wherein the resistance is conferred by a transgene comprising SEQ ID NO: 11, or a sequence at least 80% identical thereto.
99. A transgenic plant, plant part, or plant cell having resistance to Asian Soybean Rust, wherein the resistance is conferred by a transgene comprising SEQ ID NO: 11, or a sequence at least 90% identical thereto.
100. A transgenic plant, plant part, or plant cell having resistance to Asian Soybean Rust, wherein the resistance is conferred by a transgene comprising a nucleotide sequence encoding the amino acid sequence of SEQ ID NO: 12, or an amino acid sequence at least 90% identical thereto.
101. A transgenic plant, plant part, or plant cell having resistance to Asian Soybean Rust, wherein the resistance is conferred by a transgene comprising SEQ ID NO: 13, or a sequence at least 70% identical thereto.
102. A transgenic plant, plant part, or plant cell having resistance to Asian Soybean Rust, wherein the resistance is conferred by a transgene comprising SEQ ID NO: 13, or a sequence at least 80% identical thereto.
103. A transgenic plant, plant part, or plant cell having resistance to Asian Soybean Rust, wherein the resistance is conferred by a transgene comprising SEQ ID NO: 13, or a sequence at least 90% identical thereto.
104. A transgenic plant, plant part, or plant cell having resistance to Asian Soybean Rust, wherein the resistance is conferred by a transgene comprising a nucleotide sequence encoding the amino acid sequence of SEQ ID NO: 14, or an amino acid sequence at least 90% identical thereto.
105. A transgenic plant, plant part, or plant cell having resistance to Asian Soybean Rust, wherein the resistance is conferred by a transgene comprising SEQ ID NO: 15, or a sequence at least 70% identical thereto.
106. A transgenic plant, plant part, or plant cell having resistance to Asian Soybean Rust, wherein the resistance is conferred by a transgene comprising SEQ ID NO: 15, or a sequence at least 80% identical thereto.
107. A transgenic plant, plant part, or plant cell having resistance to Asian Soybean Rust, wherein the resistance is conferred by a transgene comprising SEQ ID NO: 15, or a sequence at least 90% identical thereto.
108. A transgenic plant, plant part, or plant cell having resistance to Asian Soybean Rust, wherein the resistance is conferred by a transgene comprising a nucleotide sequence encoding the amino acid sequence of SEQ ID NO: 16, or an amino acid sequence at least 90% identical thereto.
109. A transgenic plant, plant part, or plant cell having resistance to Asian Soybean Rust, wherein the resistance is conferred by a transgene comprising SEQ ID NO: 17, or a sequence at least 70% identical thereto.
110. A transgenic plant, plant part, or plant cell having resistance to Asian Soybean Rust, wherein the resistance is conferred by a transgene comprising SEQ ID NO: 17, or a sequence at least 80% identical thereto.
111. A transgenic plant, plant part, or plant cell having resistance to Asian Soybean Rust, wherein the resistance is conferred by a transgene comprising SEQ ID NO: 17, or a sequence at least 90% identical thereto.
112. A transgenic plant, plant part, or plant cell having resistance to Asian Soybean Rust, wherein the resistance is conferred by a transgene comprising a nucleotide sequence encoding the amino acid sequence of SEQ ID NO: 18, or an amino acid sequence at least 90% identical thereto.
113. A transgenic plant, plant part, or plant cell having resistance to Asian Soybean Rust, wherein the resistance is conferred by a transgene comprising SEQ ID NO: 19, or a sequence at least 70% identical thereto.
114. A transgenic plant, plant part, or plant cell having resistance to Asian Soybean Rust, wherein the resistance is conferred by a transgene comprising SEQ ID NO: 19, or a sequence at least 80% identical thereto.
115. A transgenic plant, plant part, or plant cell having resistance to Asian Soybean Rust, wherein the resistance is conferred by a transgene comprising SEQ ID NO: 19, or a sequence at least 90% identical thereto.
116. A transgenic plant, plant part, or plant cell having resistance to Asian Soybean Rust, wherein the resistance is conferred by a transgene comprising a nucleotide sequence encoding the amino acid sequence of SEQ ID NO: 20, or an amino acid sequence at least 90% identical thereto.

1. An isolated, recombinant, or synthetic polynucleotide comprising a nucleic acid sequence encoding a functional FIT1 protein homologous to SEQ ID NO: 2, wherein said homologous protein is at least 70% identical to SEQ ID NO: 2.

2. The isolated, recombinant, or synthetic polynucleotide of claim 1, wherein the functional FIT1 protein is selected from a plant of genera consisting of Abrus, Mucuna, Cajanus, Lablab, Phaseolus, and Vigna.

3. The isolated, recombinant, or synthetic polynucleotide of claim 1, wherein the nucleic acid encodes a protein selected from the group consisting of: SEQ ID NOs: 2, 4, 6, 8, 10, 12, 14, 16, 18, 20, and proteins at least 90% identical thereto.

4. The isolated, recombinant, or synthetic polynucleotide of claim 1, wherein the polynucleotide comprises a nucleic acid sequence selected from the group consisting of SEQ ID NOs: 1, 3, 5, 7, 9, 11, 13, 15, 17, 19 complements thereof, fragments thereof, and sequences at least 70% identical thereto.

5. A genetic construct comprising the nucleic acid sequence of claim 1.

6. A transgenic plant, plant part, or plant cell having resistance or tolerance to a pathogen, wherein the resistance or tolerance is conferred by a transgene comprising the nucleic acid sequence of claim 1.

7. The transgenic plant, plant part, or plant cell of claim 6, wherein the pathogen is a fungus from the order Cantharellales or Pucciniales.

8. The transgenic plant, plant part, or plant cell of claim 7, wherein the fungal pathogen is Rhizoctonia solani, Melampsora spp., Phakopsora pachyrhizi, Phakopsora meibomiae, Phakopsora euvitis, Phakopsora spp., Puccinia spp., Uromyces spp., Austropuccinia spp., Cronartium spp., Austropuccinia spp., Cronartium spp. or Hemileia vastatrix.

9. The transgenic plant, plant part, or plant cell of claim 6, wherein the plant, plant part, or plant cell is in the subfamily Papilionoideae.

10. The transgenic plant, plant part, or plant cell of claim 6, wherein the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

11. The transgenic plant, plant part, or plant cell of claim 10, wherein the plant, plant part, or plant cell is Glycine max, and wherein the plant, plant part, or plant cell is resistant to Asian Soybean Rust caused by Phakopsora pachyrhizi.

12. The transgenic plant, plant part, or plant cell of claim 11, wherein the resistance to Asian Soybean Rust is conferred by a transgene comprising SEQ ID NO: 5, or a sequence at least 70% identical thereto.

13. The transgenic plant, plant part, or plant cell of claim 11, wherein the resistance to Asian Soybean Rust is conferred by a transgene comprising a nucleotide sequence encoding the amino acid sequence of SEQ ID NO: 6, or an amino acid sequence at least 90% identical thereto.

14. A method of producing a plant, plant part, or plant cell having resistance or tolerance to a pathogen, wherein the method comprises:
transforming a plant, plant part, or plant cell with an isolated, recombinant, or synthetic polynucleotide comprising:
a nucleic acid sequence encoding all or a substantial fragment of a functional FIT1 protein, wherein the protein is at least 70% identical to a protein selected from the group consisting of: SEQ ID NOs: 2, 4, 6, 8, 10, 12, 14, 16, 18, and 20, or
an isolated, recombinant, or synthetic polynucleotide encoding a functional FIT1 protein, wherein the nucleic acid sequence is selected from the group consisting of SEQ ID NOs: 1, 3, 5, 7, 9, 11, 13, 15, 17, 19 complements thereof, fragments thereof, and sequences at least 70% identical thereto; and
selecting a plant comprising the polynucleotide and having resistance or tolerance to a pathogen.

15. The method of claim 14, wherein the nucleotide sequence encoding the FIT1 protein has been codon optimized.

16. The method of claim 14, wherein the plant, plant part, or plant cell is transformed with two or more polynucleotides encoding different FIT1 proteins.

17. The method of claim 14, wherein the plant, plant part, or plant cell is transformed or introgressed with one or more additional desired traits.

18. The method of claim 17, wherein the one or more additional desired traits are resistance traits to a disease, pest, or abiotic stress.

19. A plant, plant part, or plant cell produced by the method of claim 14, wherein the plant, plant part, or plant cell is resistant or tolerant to a pathogen.

20. The plant, plant part, or plant cell of claim 19, wherein the FIT1 protein is transiently expressed.

21. The plant, plant part, or plant cell of claim 19, wherein the FIT1 protein is stably expressed.

22. The plant, plant part, or plant cell of claim 19, wherein the plant, plant part, or plant cell is in the subfamily Papilionoideae.

23. The plant, plant part, or plant cell of claim 19, wherein the plant, plant part, or plant cell is Alysicarpus spp., Astragalus spp., Baptisia spp., Cajanus spp., Calopogonium spp., Caragana spp., Centrosema spp., Cologania spp., Crotalaria spp., Desmodium spp., Genista spp., Glycine spp., Glycyrrhiza spp., Indigofera spp., Kummerowia spp., Lablab spp., Lathyrus spp., Lespedeza spp., Lotus spp., Lupinus spp., Macroptilium spp., Macrotyloma spp., Medicago spp., Neonotonia spp., Pachyrhizus spp., Pisum spp., Phaseolus spp., Pseudovigna spp., Psoralea spp., Robinia spp., Senna spp., Sesbania spp., Strophostyles spp., Tephrosia spp., Teramnus spp., Trifolium spp., Vicia spp., Vigna spp., or Voandzeia spp.

24. The plant, plant part, or plant cell of claim 19, wherein the plant is Glycine max, wherein the plant, plant part, or plant cell is resistant to Asian Soybean Rust caused by Phakopsora pachyrhizi.

25. The transgenic plant, plant part, or plant cell of claim 18, wherein the resistance to Asian Soybean Rust is conferred by a transgene comprising SEQ ID NO: 5, or a sequence at least 70% identical thereto.

26. The transgenic plant, plant part, or plant cell of claim 19, wherein the resistance to Asian Soybean Rust is conferred by a transgene comprising a nucleotide sequence encoding the amino acid sequence of SEQ ID NO: 6, or an amino acid sequence at least 90% identical thereto.

27. A method of genetically engineering a pathogen resistance or tolerance trait in a plant, plant part, or plant cell, comprising:
providing a plant species that is susceptible to a pathogen;
identifying within the genome of the plant species a homolog of FIT1, wherein said homolog does not mediate AvrFIT1 recognition; and
genetically modifying a plant, plant part, or plant cell from the susceptible plant species with targeted gene editing, wherein said targeted gene editing is directed at the FIT1 homolog, and wherein said targeted gene editing enables the FIT1 homolog to recognize AvrFIT1 and confers resistance or tolerance to a pathogen.

28. The method of claim 27, wherein the targeted gene editing uses an engineered or natural nuclease selected from the group consisting of homing endonucleases/meganucleases (EMNs), zinc finger nucleases (ZFNs), and transcription activator-like effector nucleases (TALENs).

29. The method of claim 27, wherein targeted gene editing uses a clustered regularly interspaced short palindromic repeats (CRISPR)-Cas nuclease selected from the group consisting of Cas9, Cas12, Cas13, CasX, and CasY.

30. A genetically modified plant, plant part, or plant cell produced by the method claim 27, wherein said plant, plant part, or plant cell exhibits resistance or tolerance to a pathogen.


SEQUENCE LISTING (34 sequences)

SEQ ID NO: 1 - DNA, 3192 bp, Vigna unguiculata
  source 1..3192: mol_type = other DNA
atggctgtgccatcattctccttttccttcacctatgacgttttcctgagcttcagaggagaagacactcgttacggtttcaccggaaatctttacagagcccttcgtgacagaggaattcacaccttcatcgacgacgaggagcttcgcaaaggagacgaaatcacctctgcacttgagaaggcaatcgaaggttctagaattttcatcatcgttttctctctcaactacgcctcttcctctttttgcttgaacgagctcgcctacatccttccccacgctaagagaaatgctttgctggttttgccactcttctacgacgtcgttccttcccacgtgcgacaccacacgggtagctttggagaagcattggatgctcatgaaaagaggttcagaggtatgagtcagggttttgagcttaacattgagaagctcaacaaatggaagatggctctgcgtcgagcagctaacttgtctggctatcatttcaaacatggggaggaatatgaataccagtttatcgagaggatagttgaattggtctctaagaagattaaccgtgctcctttacacgttggggattatccagttggactagaggcgcgggtgctagaagtaaagttgcttctagaagtaggatctgatgatgttgtccacttggtagggatccatggactcggtggaatcggtaaaaccacacttgctcttgcactctacaattccatcgctgaccattttgaaggtttgtgtttcctcgaaaatgtgagagagaattcaaacaaacacggcctacatcatcttcaaagaatccttctttctcaagtgcttggagaaaataatatcaacataactagtgtgagacaaggaatttcaatgatgcagcataggctacggcagaagaagattctcttgattctagatgatgttgacaagcacgaacagttacaggcaattgttggaagacctgattggtttggtcccggcagtagagtcatcatcacaaccagggacaaacagttgttgtcgtgtcacttggttgaaaaattatataaagtgaagaagttggaaaagaacaatgctcttcgactgcttagttggaaaggttttagaacagaagaagttgatacaagttatttgaacgtcatggatcgtgtactagcttatgcttctggacatccattggctttggaagtaataggttcgaagttgtttagtaaaagtgtaaaagaatgggaatctgccatcaaacagtacgagaaaattcctagcaatcaaatccttgaggtacttaaagtaagttttgatgctttagaggaagtagagaagagtgtttttcttgacattgcttgttgtttcaaaggatatgcattgtcagagatagaagatattcttcgtgctcattatggtgattgcatgaaatatcatattggagtgttagttgaaaaatctctcatgaaatatggctataattctgtagttacgttgcatgacttgatagaagacatggggaaagaaattgtccgagagaaatcaccaaaaaatccagggaagcgcagtagattatggtcaccagaagatataattcaagttttggaagacaattcgggaagtggagaaattgaaatcatatgtttgaattcctccttacctgacaaagaagaaatagtaggatggaacagaaaggccttcaaaaagatgaaaaacctcaaaacacttatcattaaaaaaggtaaattttcggaaggtcctaaatatcttccaaatagtttaagagtacttgaatggttgaaatatccttcacaagggctaccaccagattttcgttcaaagaaacttgccatatgcaaattaccttcaagtagttttgggtcactcgaattggctgagttttcaaagaagttcatgaatatgactcttctgaattttgatgaatgtgaaggtttaacacatatacctgatgtgtctgggctgccaaatttagaaaaagtttcattcaagaattgtaagagtttagttacaatccatgactccttcgggttcctaggtaagcttaactccttgagtgctgttggttgcagcaagcttaggagttttcctcccctcaaattgacttctctggaaaatcttgaactttcatattgtcacagtctcgagagcttcccagaaatattagaaaaaatgggaaagataacagaacttgtcttggaggattgtcacataaaagaattaccattttcatttcataatctcaccgagcttcaaacattacagttgcgttggtgtccaatattaaggttaccaagtagtattgtcatgatgccaaaactggcccagattattgcttgggaatctaaaggatggctatttccaaaacaggttgatggggaagaaaaaggaagctcaatggtgtcttcaaatgtagattgtcttgttctctcagggtgcaaactctcagatgactttttcccagtaattcctgaatggttttctaatgtaaaagatttagacctgtcaaggaataatttcacggttcttcctgaatgcatcagcaattgtcattctttatgcaagcttactttagatagctgtcatagtcttcaagagattcgagggattccacctaacatacgacatttatcagcaagaaattgtaaatccttcacttcctcttgcagaagcactttactgaatcagaaactgcatgaggctgggaacaccatgttttggttgtcaggagcaaagtttccagaatggttcgatcaccacggtcggggaccatcttgttctttctgggttggcaacaaattcccttcaattgctctttgtattgctattggacaaactcatatcgaacaagttgaaatcgttggacctatcatgatcatcaacggcattgaatgttcatttgatgaggaggaggatccttatttatatatgctccctcatcacacacatattttcgatttgcaacatatagttttttcagattatctagacagatatgtttcagaaaatgaatggaaccatgtggagatcacatactcagtggagcagagattcaataaaaaagacaaacatgcggtgaccccaatctctatagaaaatggaatctatgtgttgaaacagaggagcagcatggaggatattcaattcactgatccccacaaaaagagaagattagatgttgtctaa

SEQ ID NO: 2 - protein, 1063 aa, Vigna unguiculata
MAVPSFSFSFTYDVFLSFRGEDTRYGFTGNLYRALRDRGIHTFIDDEELRKGDEITSALEKAIEGSRIFIIVFSLNYASSSFCLNELAYILPHAKRNALLVLPLFYDVVPSHVRHHTGSFGEALDAHEKRFRGMSQGFELNIEKLNKWKMALRRAANLSGYHFKHGEEYEYQFIERIVELVSKKINRAPLHVGDYPVGLEARVLEVKLLLEVGSDDVVHLVGIHGLGGIGKTTLALALYNSIADHFEGLCFLENVRENSNKHGLHHLQRILLSQVLGENNINITSVRQGISMMQHRLRQKKILLILDDVDKHEQLQAIVGRPDWFGPGSRVIITTRDKQLLSCHLVEKLYKVKKLEKNNALRLLSWKGFRTEEVDTSYLNVMDRVLAYASGHPLALEVIGSKLFSKSVKEWESAIKQYEKIPSNQILEVLKVSFDALEEVEKSVFLDIACCFKGYALSEIEDILRAHYGDCMKYHIGVLVEKSLMKYGYNSVVTLHDLIEDMGKEIVREKSPKNPGKRSRLWSPEDIIQVLEDNSGSGEIEIICLNSSLPDKEEIVGWNRKAFKKMKNLKTLIIKKGKFSEGPKYLPNSLRVLEWLKYPSQGLPPDFRSKKLAICKLPSSSFGSLELAEFSKKFMNMTLLNFDECEGLTHIPDVSGLPNLEKVSFKNCKSLVTIHDSFGFLGKLNSLSAVGCSKLRSFPPLKLTSLENLELSYCHSLESFPEILEKMGKITELVLEDCHIKELPFSFHNLTELQTLQLRWCPILRLPSSIVMMPKLAQIIAWESKGWLFPKQVDGEEKGSSMVSSNVDCLVLSGCKLSDDFFPVIPEWFSNVKDLDLSRNNFTVLPECISNCHSLCKLTLDSCHSLQEIRGIPPNIRHLSARNCKSFTSSCRSTLLNQKLHEAGNTMFWLSGAKFPEWFDHHGRGPSCSFWVGNKFPSIALCIAIGQTHIEQVEIVGPIMIINGIECSFDEEEDPYLYMLPHHTHIFDLQHIVFSDYLDRYVSENEWNHVEITYSVEQRFNKKDKHAVTPISIENGIYVLKQRSSMEDIQFTDPHKKRRLDVV

SEQ ID NO: 3 - DNA, 3201 bp, Vigna radiata
  source 1..3201: mol_type = other DNA
atggctgtggcatcattctcttcttccttcacctatgacgtgttcctcagcttcagaggagaagatactcgttacagtttcaccggcaatctctacagagcccttcgtgacagaggaattcacaccttcatcgacgacgagaagcttcccaaaggagacgaaatcacctctgcacttgagaaggcaatagaaggttccagaattttcatcatcgttttctctcgcaactacgcctcttcctccttttgcttgaacgagctcgcctacattcttccctacgctaatagaaatggtttgctggttttgccactcttctacgacgtcgttccttcccacgtgcgccaccacatgggtagctatggagaagctttggatactcatgaaaacaggttcaaagctacgagtcagggttttgagcttaacatggagaagctcaacaaatggaagatggctctgcgtggaacagctaacttatctggctatcatttcaaacatggggaggaatatgaatacgagtttatcaagaggatagttgacttggtctccaacaagattaaccgtgctcctttacacgttgcggattatccagttggactagagactcgagtgctagaagtaaagttgcttctggatataggatctgatgatggtgtccacatggtagggatccatggactcggtggagttggtaaaaccacacttgctcttgcagtttacaattccatcgctgaccattttgaaggcttgtgtttcctcgaaaatgtgagagagaattcaaacaaacacggcctacagcatcttcaaagaatccttctttctcaaatggttggagaaaataatgtcaacataactagtgtgagacaaggaatttcaatgatgcagcataggctacgacagaagaagattctcttgattttagatgacgttgacaaacatgaacagttacaagctattgttggaagacctgattggtttggtcccggcagtagagtcatcatcacaactagggacaaacatttgttgtcatgtcacttgatcgaaaaattatataaagtgaagaagttgaaaaagaacaatgctcatcgactgcttagttggaaagcttttagaacagaagaagttgatacaagttatttgaatgtaatggatcgtgtactagcttatgcttctggccatccattggctttggaagtaatcggttcgaagttgtttagaaaaagtgtaaaggaatgggaatctgccatcaaacagtatgagaaaattcctaacaatcaaatcctggaggtgcttaaaataagttttgatgctttagaggaagtagagaagagtgtttttcttgacatttcttgttgctttaaaggatatgcattgtcagaggtggaagatatacttcgtgctcattatggtgattgcatgaaatatcatattggggtgttggttgaaaagtctctcataaaatatggttggagttgtgtggttacaatgcatgacttgatagaagacatgggtaaagaaattgtccggcagaaatctccaaataggccagggaagcgcagtagattatggtcaccagaagagataattaaagttttggaagacaacttggggagtggagaaattgaaatcatatgtttaaattcctccttacctgacaaagaagaaatagtagaatggaacagaaaggccttcaagaagatgaaaaacctcaaaacacttatcattaaaaatggtaatttttcggaaggtcctgaatatcttccaaatagtttaagagtactggaatggttgaaatatccttcacatgggctaccgccagattttcgttcaaagaaactttccttatgcaaattaccttcaagttgttttgggtcactcgaattggctgagttttcaaagaagttcatgaatatgactcttctgaattttgacgaatgtgaaggtttaacacagatacctgatctatctgggatgccaaatttagaaagattttcattcaagaattgtaagagtttaattacaattcatgactccattggcttcctaggtaagcttaactccttgaatgctgttggttgcagcaagcttaggagttttccccccctcaaattgacttctctggaaaatcttgaactttcatattgttacagtcttgagagcttcccagaaatattaggaaaaatgggacagataacagaacttgtcttggaggactgtcacataaaagaattgccaatttcatttcaaaatctcaccgagcttcgaacattacagttgcgttcgtgtccaacgttaaggttacctagtagtattgtcatgatgccaaaactggccaacattattgcttgggaatctaaagggtggctatttccaaaacaggtcgagagggaagagaaaataggctcaatggtgtcttcaaacgtagattgtcttgttctctcagggtgcaaactctcggatgactttttcccaataattcttgaatggtttgctaatgtaaaagatttaaacctatcaaggaataacttcacggttcttcctgagtgcatcgccaattgtcacttgttatgcaagcttactttagatgcctgtcatagtcttcgagagattagagggattccaccaaacatacgacagttactagcaagaaattgtaaatccttcacttcttcttgcagaagaactttactgaatcagaaactgcatgaggctggaaacaccatgttttcgttttcaggagcaaggtttccagaatggttcgatcaccacagtcggggagcatcttgttctttctgggttggcaaaaaattcccttccattgctctttgtattgctattggaccaactcatttggaacaccttgaaattgttggacctatcatgatcatcaactgcattgaatgttcatttgattgggaggagaatccttatttatatatgctccctcatcacacatatattttcgatttgcaaaatatagtttttccagattatctagacagatttgtttcagaaaatgaatggaaccacgtggagattacatactcagtcaagcagagattcaagggaaaagacaaacatgaggtgaccccaatctctatagaaaatggaatctatgtgttcaaacagaagagcagcatggaggatattcaattcactgatccccacaaaaagagaagattagatgttgatccagagttgtaa

SEQ ID NO: 4 - protein, 1066 aa, Vigna radiata
MAVASFSSSFTYDVFLSFRGEDTRYSFTGNLYRALRDRGIHTFIDDEKLPKGDEITSALEKAIEGSRIFIIVFSRNYASSSFCLNELAYILPYANRNGLLVLPLFYDVVPSHVRHHMGSYGEALDTHENRFKATSQGFELNMEKLNKWKMALRGTANLSGYHFKHGEEYEYEFIKRIVDLVSNKINRAPLHVADYPVGLETRVLEVKLLLDIGSDDGVHMVGIHGLGGVGKTTLALAVYNSIADHFEGLCFLENVRENSNKHGLQHLQRILLSQMVGENNVNITSVRQGISMMQHRLRQKKILLILDDVDKHEQLQAIVGRPDWFGPGSRVIITTRDKHLLSCHLIEKLYKVKKLKKNNAHRLLSWKAFRTEEVDTSYLNVMDRVLAYASGHPLALEVIGSKLFRKSVKEWESAIKQYEKIPNNQILEVLKISFDALEEVEKSVFLDISCCFKGYALSEVEDILRAHYGDCMKYHIGVLVEKSLIKYGWSCVVTMHDLIEDMGKEIVRQKSPNRPGKRSRLWSPEEIIKVLEDNLGSGEIEIICLNSSLPDKEEIVEWNRKAFKKMKNLKTLIIKNGNFSEGPEYLPNSLRVLEWLKYPSHGLPPDFRSKKLSLCKLPSSCFGSLELAEFSKKFMNMTLLNFDECEGLTQIPDLSGMPNLERFSFKNCKSLITIHDSIGFLGKLNSLNAVGCSKLRSFPPLKLTSLENLELSYCYSLESFPEILGKMGQITELVLEDCHIKELPISFQNLTELRTLQLRSCPTLRLPSSIVMMPKLANIIAWESKGWLFPKQVEREEKIGSMVSSNVDCLVLSGCKLSDDFFPIILEWFANVKDLNLSRNNFTVLPECIANCHLLCKLTLDACHSLREIRGIPPNIRQLLARNCKSFTSSCRRTLLNQKLHEAGNTMFSFSGARFPEWFDHHSRGASCSFWVGKKFPSIALCIAIGPTHLEHLEIVGPIMIINCIECSFDWEENPYLYMLPHHTYIFDLQNIVFPDYLDRFVSENEWNHVEITYSVKQRFKGKDKHEVTPISIENGIYVFKQKSSMEDIQFTDPHKKRRLDVDPEL

SEQ ID NO: 5 - DNA, 3195 bp, Phaseolus lunatus
  source 1..3195: mol_type = other DNA
atgtctgtgtcatcattctcctcttccttcacctatgacgtcttcctcagcttcagaggagaagacactcgttacggtttcaccggaaatctctattcagcccttcgtgacggcggaatttgcaccttcatcgatgacgaggagctccccaaaggagacgaaatcaccactgcacttgagaaggctatcgaaggttccagaattttcatcatcgttttctctcacaactacgcatcttcttccttttgcttgaatgagctcgcctacattcttccctacgctaagagaaatggtttgctggttttgccactcttctacgacgtcgttccttcccacgtgcgaaaccacacgggtacctttggagaagcgttagatactcatgaaaacaggttcaaagctacgagtgagggttttgagcttaacatggagaagctcaacagatggaagatggctctgcgtggagctgctaacttatctggctatcatttcaaacatggggaggaatatgaataccagtttatcaagaggatagtggaattggtctctaacaagattaaccgtgctcctttacatgttgcggattatccagttggactagagacacgagtgctagaagtaaaattgcttctagatgtaggatctgatgatggtgtccacatggtagggatccatggattcggtggagttggtaaaaccacacttgctcttgcagtttacaattccatagctgacaattttgaaggcttgtgtttccttgaaaatgtcagagagaattcaaacaaacacggcctacaacatcttcagagtctccttctttctcaaatggttggagaaaataatatcaacataactagtgtgaaacaaggaatttcaatgatgcagcatagactacggcagaagaagattcttttgattcttgatgatgttgacaagcatgaacagttacaagcaattgttggaagagctgattggtttggtcccggcagtagagtgatcatcacaactagggacaaacatttgttgtcatgtcagttggttgaaaaaatatatgaagtgaagaagttgaaaaagaaagatgctcttcgactgcttagttggaaaggttttaaaacagaagaagttcatccaagttatgtgaatgtcatggatcgtgtactggcttatgcttctggccatccattggctttggaagtaataggttcgaagttgtttagaaaaagtgtaaaagaatgggaatctgccatccaccagtatgagaaaattcctaacaatcaaattctggagatgcttaaaataagttttgattctttagaggaagtagagaagagtgtttttcttgacattgcttgttgtttcaaaggatatgcgttgtcagaggtggaaaatatacttcgtgctcattatggtgattgcatgaaatatcatattggggtgttggttgaaaaatctctcataaaatatggatggaattctgtagttacattgcatgacttgatagaagacatgggtaaagaaattgtccgacagaaatcaccaaataagccagggaagcgcagtagattatggtcaccagaagatataattcaagttttggaagacaactcgggaagtggagaaattgaaatcatatgtttgaattactctttacctgacaaagcagaaatagtagaatggaacagaaaggccttcaagaagatgaaaaacctcaaaacacttatcattaaaagtggtaaattttcggaaggtcctaagtatcttccaaacagtttaagagtaatggaatggttaaaatatccttcacaagggctaccgccagattttcgttcaaaggaacttgccatatgcaaattacctgcaagttgttttgggtctctcgaattggccgagttatcaaagaagttcaagaatatgacccttttgaattttgacgaatgtgaaggtttaacacagatacctgatgtatctgggctgccaaatttagtagaaatttcattcaagaattgtaagagtttaattacaatccatgactccattgggttcctaggtaagcttatctccttgaatgctgttggttgcagcaagcttaggagtttcccccccctcaaattgacttctctggaaaatcttgaactttcatattgttacagtcttgagagcttcccagaaatattgggaaaaatgggaaagataacagaacttttgttggagtgctgtgacataaaagaattgccattttcatttcaaaatctcactgagcttcaaacattacagttgcgttactgtccaatgttgaggttgccaagtagtattgtcatgatgccaaaactgaccgagattattgcttgggaatctaaaggatggctatttccaaaacaggttgagggtgaagagaaagtaagctcaatggtgtcttcaaatgtagattgtcttcttctcccagggtgcaaactctcggatgattttttcccaataacatggtttgctaatgtaaaagagttagacctgtcaaggaatactttcacggttcttcctgaatgcatcagcaattgtcattttttatgtaagcttattttagataactgtcagagtcttcgagagattagagggattccgccaaacgtacgacatttatcagcaagatattgtaaatccttcacttcttcttgcagaagcactttactgaatcagaaactgcatgaggcgggaaacaccatgttttggttttcaggagcaagttttccagaatggttcgagcaccacagtcggggaccatcttgttctttctgggttgacaagaaattccctgccattgctctttgtattgttattggaccaactcatttggaacatactgagattgttggacctatcataatcatcaatggcgttgattgttcacttgttgatgagttggataatccttatttacggatactccctcatcacacatatcttttcgatttgcaacatatagtttcttcagattatctagacagatttgtttcagaaaaggaatggaatcacgtggagattagatactcagtagagcagagattcagtgaagaagacaaacatgtggtgataccagtctccgtagaaaatggaatctatgtgttcaaacagaggagcagcatggaggatattcaattcactgatccccacaaaaagagaagattagatgttgatccagagtag

SEQ ID NO: 6 - protein, 1064 aa, Phaseolus lunatus
MSVSSFSSSFTYDVFLSFRGEDTRYGFTGNLYSALRDGGICTFIDDEELPKGDEITTALEKAIEGSRIFIIVFSHNYASSSFCLNELAYILPYAKRNGLLVLPLFYDVVPSHVRNHTGTFGEALDTHENRFKATSEGFELNMEKLNRWKMALRGAANLSGYHFKHGEEYEYQFIKRIVELVSNKINRAPLHVADYPVGLETRVLEVKLLLDVGSDDGVHMVGIHGFGGVGKTTLALAVYNSIADNFEGLCFLENVRENSNKHGLQHLQSLLLSQMVGENNINITSVKQGISMMQHRLRQKKILLILDDVDKHEQLQAIVGRADWFGPGSRVIITTRDKHLLSCQLVEKIYEVKKLKKKDALRLLSWKGFKTEEVHPSYVNVMDRVLAYASGHPLALEVIGSKLFRKSVKEWESAIHQYEKIPNNQILEMLKISFDSLEEVEKSVFLDIACCFKGYALSEVENILRAHYGDCMKYHIGVLVEKSLIKYGWNSVVTLHDLIEDMGKEIVRQKSPNKPGKRSRLWSPEDIIQVLEDNSGSGEIEIICLNYSLPDKAEIVEWNRKAFKKMKNLKTLIIKSGKFSEGPKYLPNSLRVMEWLKYPSQGLPPDFRSKELAICKLPASCFGSLELAELSKKFKNMTLLNFDECEGLTQIPDVSGLPNLVEISFKNCKSLITIHDSIGFLGKLISLNAVGCSKLRSFPPLKLTSLENLELSYCYSLESFPEILGKMGKITELLLECCDIKELPFSFQNLTELQTLQLRYCPMLRLPSSIVMMPKLTEIIAWESKGWLFPKQVEGEEKVSSMVSSNVDCLLLPGCKLSDDFFPITWFANVKELDLSRNTFTVLPECISNCHFLCKLILDNCQSLREIRGIPPNVRHLSARYCKSFTSSCRSTLLNQKLHEAGNTMFWFSGASFPEWFEHHSRGPSCSFWVDKKFPAIALCIVIGPTHLEHTEIVGPIIIINGVDCSLVDELDNPYLRILPHHTYLFDLQHIVSSDYLDRFVSEKEWNHVEIRYSVEQRFSEEDKHVVIPVSVENGIYVFKQRSSMEDIQFTDPHKKRRLDVDPE

SEQ ID NO: 7 - DNA, 3195 bp, Phaseolus vulgaris
  source 1..3195: mol_type = other DNA
atggctgtgccatcattctcctcttccttcacctacgacgtcttcctcagcttcagaggagaagatactcgttacggtttcaccggcaatctctatacagcccttcatgacagcggaatttgcaccttcatcgacgacgaggagctccccaaaggagacgaaatcaccacctcactacagaaggcaatcgaagcttccagaattttcatcatcgttttctctcacaactacgcatcttcttccttttgcttgaacgagctcgcctacattcttccctacgctaagagaaatggtttgctggttttgccactcttctacgacgtcgttccttcccacgtgcgacaccacacgggtacctttggagaagcgttagatactcatgaaaacaagttcaaaggtacgagtgagggttttgagcttaacatggagaagctcaacagatggaaaatggctctgcgtggagctgctaacttatctggctatcatttcaaacatggggaggaatatgaataccagtttatcaagaggatagtgaaattggtctctaacaagattaaccgtgctactttacatgttgcggattatccagttggactagagacacgaatgctagaagtaaagttgcttctagatgtaggatctgatgatggtgtccacatggtagggatccatggattcggtggagttggtaaaaccacacttgctcttgcagtttacaactccatagctgacaattttgaaggcttgtgtttccttgaaaatgtgagagagaattcaaacaaacacggcctacaacatcttcagagcctccttctttctcaaatggttggagaaaataatatcaacataactagtgtgaaacaagggatttcaatgatgcagcataggctacggcagaagaagattcttttgattcttgatgatgttgacaagcatgaacagttacaagcaattgttggaagagctgattggtttggtcccggcagtagagtcatcatcacaactagggacaaacatttgttgtcatgtcagttggttgaaaaaatatatgaagtgaagaagttgaaaaagaaagatgctcttcgactgcttagttggaaaggttttaaaacagaagaagttcatccaagttatgtgaatgtcatgggtcgtgtactggcttatgcttctggccatccattggctttggaagtaataggttcgaagttgtttagaaaaagtgtaaaagaatgggaatctgccatccagcagtatgagaaaattcctaacaatcaaatcctggagatacttaaaataagttttgattcattagaggaagtagagaagagtgtttttcttgacattgcttgttgtttcaaaggatatgcattgtcagaggtggaaaatatacttcgtgctcattatggtgattgcatgaaatatcatattggagtgttggttgaaaaatctctcataaaatatagatggaattctgtagttacactgcatgacttgatagaagacatgggtaaagaaattgtccgacagaaatcaccaaataaaccggggaagcgcagtagattatggtcaccagaagatataattcaagttttggaagacaactcgggaagtggagaaattgaaatcatatgtttgaattactctttacctggcaaagaagaaatagtagaatggaacagaaaggccttcaagaagatgaaaaacctcaaaacacttattattaaaagtggtaatttttcggaaggtcctaaatatcttccaaacagtttaagagtaatggaatggttaaaatatccttcacaagggctaccgccagattttcgttcaaaggaacttaccatatgcaaattacctgcaagttgttttgggtccctcgaattggccgagttatcaaagaagttcatgaatatgacccttttgaattttgacgaatgtgaaggtttaacacagatacctgatgtatctgggctgccaaatttagtagaaatttcattcaagaattgtaagagtttaattacaatccatgactccattgggttcctaggtaagcttaactccttgaatgctgttggttgcagcaagcttaggagttttccacccctcaaattgacttctctggaaaatcttgaactttcatattgttacagtcttgagagcttcccagaaatattaggaaaaatgggaaagataacagaacttttcttggagtgctgtgacataaaagaattgccattttcatttcaaaatctcactgagcttcaaacattacagttgcgttactgtccaatgttaaggttgccaagtagtattgtcatgatgccaaaactgaccgagattattgcttgggaatctgaaggatggctatttccaaaacaggttgagggtgaagagaaagtaagctcaatggtgtcttcaaatgtagattgtcttcttctcccagggtgcaaactctcagatgattttttcccaataacatggtttgctaatgtaaaagagttagacctgtcaaggaatactttcacggttcttcctgaatgcataagcaattgttattttttatgtaagcttattttagataactgtcatagtcttcgagagattagagggattccgccaaacatacgacatttatcagcaagatattgtaaatccttcacttcttcttgcagaagcactttactgaatcagaaactgcatgagacgggaaacaccatgttttggttttcaggagcaagttttccagaatggttcgagcaccgcagtcagggaccatcttgttctttctgggttggcaacaaattccctgccattgctctttgtattgttattggaccaactcatttggaccatattgagattgttggacctatcatgatcatcaatggcattgattgttcacttgatgatgggttggttaatccttatttatggataccccctcatcacacatatcttttcgatttgcaacatatagtttcttcagattatcttgacagatttgtttcagaaaaggaatggaatcacgtggagattagatactcagtcaagcagagattcagtgaaaaagacaaacatgtggtgataccagtctctatagaaaatggaatctatgtgttcaaacagaggagcagcatggaggatattcaattcactgatccccacaaaaagagaagattagatgttgatccagagtag

SEQ ID NO: 8 - protein, 1064 aa, Phaseolus vulgaris
MAVPSFSSSFTYDVFLSFRGEDTRYGFTGNLYTALHDSGICTFIDDEELPKGDEITTSLQKAIEASRIFIIVFSHNYASSSFCLNELAYILPYAKRNGLLVLPLFYDVVPSHVRHHTGTFGEALDTHENKFKGTSEGFELNMEKLNRWKMALRGAANLSGYHFKHGEEYEYQFIKRIVKLVSNKINRATLHVADYPVGLETRMLEVKLLLDVGSDDGVHMVGIHGFGGVGKTTLALAVYNSIADNFEGLCFLENVRENSNKHGLQHLQSLLLSQMVGENNINITSVKQGISMMQHRLRQKKILLILDDVDKHEQLQAIVGRADWFGPGSRVIITTRDKHLLSCQLVEKIYEVKKLKKKDALRLLSWKGFKTEEVHPSYVNVMGRVLAYASGHPLALEVIGSKLFRKSVKEWESAIQQYEKIPNNQILEILKISFDSLEEVEKSVFLDIACCFKGYALSEVENILRAHYGDCMKYHIGVLVEKSLIKYRWNSVVTLHDLIEDMGKEIVRQKSPNKPGKRSRLWSPEDIIQVLEDNSGSGEIEIICLNYSLPGKEEIVEWNRKAFKKMKNLKTLIIKSGNFSEGPKYLPNSLRVMEWLKYPSQGLPPDFRSKELTICKLPASCFGSLELAELSKKFMNMTLLNFDECEGLTQIPDVSGLPNLVEISFKNCKSLITIHDSIGFLGKLNSLNAVGCSKLRSFPPLKLTSLENLELSYCYSLESFPEILGKMGKITELFLECCDIKELPFSFQNLTELQTLQLRYCPMLRLPSSIVMMPKLTEIIAWESEGWLFPKQVEGEEKVSSMVSSNVDCLLLPGCKLSDDFFPITWFANVKELDLSRNTFTVLPECISNCYFLCKLILDNCHSLREIRGIPPNIRHLSARYCKSFTSSCRSTLLNQKLHETGNTMFWFSGASFPEWFEHRSQGPSCSFWVGNKFPAIALCIVIGPTHLDHIEIVGPIMIINGIDCSLDDGLVNPYLWIPPHHTYLFDLQHIVSSDYLDRFVSEKEWNHVEIRYSVKQRFSEKDKHVVIPVSIENGIYVFKQRSSMEDIQFTDPHKKRRLDVDPE

SEQ ID NO: 9 - DNA, 3195 bp, Phaseolus acutifolius
  source 1..3195: mol_type = other DNA
atggctgtgccatcattctcctcttccttcacctacgacgtcttcctcagcttcagaggagaagatactcgttacggtttcaccggcaatctctatacagcccttcatgacagcggaatttacaccttcatcgatgacgaggagctccccaaaggagacgaaatcaccacctcactacagaaggcaatcgaaggttccagaattttcatcatcgttttctctcacaactacgcatcttcttccttttgcttgaacgagctcgcctacattcttccctacgctaagagaaatggtttgctggttttgccactcttctacgacgtcgttccttcccacgtgcgacaccacacgggtacctttggagaagcgttagatactcatgaaaacaagttcaaaggtacgagtgagggttttgagcttaacatggagaagctcaacagatggaagatggctctgcgtggagctgctaacttatctggctatcatttcaaacatggggaggaatatgaataccagtttatcaagaggatagtgaaattggtctctaacaagattaaccgtgctcctttacatgttgcggattatccagttggactagagacacgaatgctagaagtaaagttgcttctagatgtaggatctgatgatggtgtccacatggtagggatccatggattcggtggagttggtaaaaccacacttgctcttgcagtttacaactccatagctgacaattttgaaggcttgtgtttccttgaaaatgtgagagagaattcaaacaaacacggcctacaacatcttcagagcctccttctttctcaaatggttggagaaaataatatcaacataactagtgtgaaacaagggatttcaatgatgcagcataggctacggcagaagaagattcttttgattcttgatgatgttgacaagcatgaacagttacaagcaattgttggaagagctgattggtttggtcccggcagtagagtcatcatcacaactagggacaaacatttgttgtcatgtcagttggttgaaaaaatatatgaagtgaagaagttgaaaaagaaagatgctcttcgactgcttagttggaaaggttttaaaacagaagaagttcatccaagttatgtgaatgtcatgggtcgtgtactggcttatgcttctggccatccattggctttggaagtaataggttcgaagttgtttagaaaaagtgtaaaagaatgggaatctgccatccaccagtatcagaaaattcctaacaatcaaatcctggagatgcttaaaataagttttgattctttagaggaagtagagaagagtgtttttcttgacattgcttgttgtttcaaaggatatgcattgtcagaggtggaaaatatacttcgtgctcattatggtgattgcatgaaatatcatattggggtgttggttgaaaaatctctcataaaatatagatggaattctgtagttacattgcatgacttgatagaagacatgggtaaagaaattgtccgacagaaatcaccaaataagccagggaagcgcagtagattatggtcaccagaagatataattcaagttttggaagacaactcgggaagtggagaaattgaaatcatatgtttgaattactctttacctgacaaagaagaaatagtagaatggaacagaaaggccttcaagaagatgaaaaacctcaaaacacttattattaaaagtggtaatttttcggaaggtcctaaatatcttccaaacagtttaagagtaatggaatggttaaaatatccttcacaagggctaccgccagattttcgttcaaaggaacttgccatatgcaaattacctgcaagttgttttgggtcactcgaattggccgagttatcaaagaagttcatgaatatgacccttttgaattttgacgaatgtgaaggtttaacacagatacctgatgtatctgggctgccaaatttagtagaaatttcattcaagaattgtaagagtttaattacaatccatgactccattgggctcctaggtaagcttaactccttgaatgctgttggttgcagcaagcttaggagttttccacccctcaaattgacttctctggaaaatcttgaactttcatattgttacagtcttgagagcttcccagaaatattagggaaaatgggaaagataacagaacttttcttggagtgctgtgacataaaagaattgccattttcatttcaaaatctcactgagcttcaaacattacagttgcgttactgtcccatgttaaggttgccaagtagtattgtcatgatgccaaaactgaccaagattattgcttgggaatctgaaggatggttatttccaaaacaggttgagggtgaagagaaagtaagctcaatggtgtcttcaagtgtagattgtcttcttctcccagggtgcaaactctcagatgattttttcccaataacatggtttgctaatgtaaaagagttagacctgtcaaggaatactttcacggttcttcctgaatgcataagcaattgttattttttatgtaagcttattttagataactgtcatagtcttcgagagattagagggattccgccaaacatacgacatttatcagcaagatattgtaaatccttcacttcttctggcagaagcactttactgaatcagaaactgcatgagacgggaaacaccatgttttggttttcaggagcaagttttccagaatggttcgagcaccgcagtcagggaccatcttgttctttctgggttgggaacaaattccctgccattgctctttgtattgttattggaccaactcatttggaccatattgagattgttggacctatcatgatcatcaatggcattgactgttcacttgttgatgggttggttaatccttatttatggataccccctcatcacacatatcttttcgatttgcaacatatagtttcttcagattatcttgacagatttgtttcagaaaaggaatggaatcacgtggagattagatactcagtcaagcagagattcagtgaaaaagacaaacatgtggtgataccagtctctatagaaaatggaatctatgtgttcaaacagaggagcagcatggaggatattcaattcactgatccccacaaaaagagaagattagatgttgatccagagtag

SEQ ID NO: 10 - protein, 1064 aa, Phaseolus acutifolius
MAVPSFSSSFTYDVFLSFRGEDTRYGFTGNLYTALHDSGIYTFIDDEELPKGDEITTSLQKAIEGSRIFIIVFSHNYASSSFCLNELAYILPYAKRNGLLVLPLFYDVVPSHVRHHTGTFGEALDTHENKFKGTSEGFELNMEKLNRWKMALRGAANLSGYHFKHGEEYEYQFIKRIVKLVSNKINRAPLHVADYPVGLETRMLEVKLLLDVGSDDGVHMVGIHGFGGVGKTTLALAVYNSIADNFEGLCFLENVRENSNKHGLQHLQSLLLSQMVGENNINITSVKQGISMMQHRLRQKKILLILDDVDKHEQLQAIVGRADWFGPGSRVIITTRDKHLLSCQLVEKIYEVKKLKKKDALRLLSWKGFKTEEVHPSYVNVMGRVLAYASGHPLALEVIGSKLFRKSVKEWESAIHQYQKIPNNQILEMLKISFDSLEEVEKSVFLDIACCFKGYALSEVENILRAHYGDCMKYHIGVLVEKSLIKYRWNSVVTLHDLIEDMGKEIVRQKSPNKPGKRSRLWSPEDIIQVLEDNSGSGEIEIICLNYSLPDKEEIVEWNRKAFKKMKNLKTLIIKSGNFSEGPKYLPNSLRVMEWLKYPSQGLPPDFRSKELAICKLPASCFGSLELAELSKKFMNMTLLNFDECEGLTQIPDVSGLPNLVEISFKNCKSLITIHDSIGLLGKLNSLNAVGCSKLRSFPPLKLTSLENLELSYCYSLESFPEILGKMGKITELFLECCDIKELPFSFQNLTELQTLQLRYCPMLRLPSSIVMMPKLTKIIAWESEGWLFPKQVEGEEKVSSMVSSSVDCLLLPGCKLSDDFFPITWFANVKELDLSRNTFTVLPECISNCYFLCKLILDNCHSLREIRGIPPNIRHLSARYCKSFTSSGRSTLLNQKLHETGNTMFWFSGASFPEWFEHRSQGPSCSFWVGNKFPAIALCIVIGPTHLDHIEIVGPIMIINGIDCSLVDGLVNPYLWIPPHHTYLFDLQHIVSSDYLDRFVSEKEWNHVEIRYSVKQRFSEKDKHVVIPVSIENGIYVFKQRSSMEDIQFTDPHKKRRLDVDPE

SEQ ID NO: 11 - DNA, 3201 bp, Vigna angularis
  source 1..3201: mol_type = other DNA
atggctgtgccatcattctcctcttccttcacctatgacgtgttcctcagcttcagaggagaagatactcgttacagtttcaccggcaatctctacagagcccttcgtgacagaggaattcacaccttcatcgacgacgagaagcttcccaaaggagacgaaatcacctctgcacttgagaaggcaatcgaaggttccagaattttcatcatcgttttctctcgcaactacgcctcttcctccttttgcttgaacgagctcgcctacattcttccctacgctaatagaaatggtttgctggttttgccactcttctacgacgtcgttccttcccacgtgcgccaccacacgggtagcttcggagaagctttggatacacatgaaaacaggttcaaagctacgagtcagggttttgagcttaacatggagaagctcaacaaatggaagatggctctgcgtggaacagctaacttatctggctatcatttcaaacatggggaggaatatgaatacgagtttatcaagaggatagttgacttggtctccaacaagattaaccgtgctcctttacacgttgcggattatccagttggactagagactcgagtgctagaagtaaagttgcttctagatataggatctgatgatggtgtccacatggtagggatccatggactcggtggagttggtaaaaccacacttgctcttgcagtgtacaattccatcgctgaccattttgaaggcttgtgtttcctcgaaaatgtgagagagaattcaaacaaacatggcctacagcatcttcaaagaatccttctttctcaaatgattggagaaaataatgtcaacataactagtgcgagacaagggatttcaatgatggagcataggctacgacagaagaagattctcttgattctagatgatgttgacaaacatgaacagttacaagcaattgttggaagacctgattggtttggtcccggcagtagagtcatcatcacaactagggacaaacatttgttatcatgtcacttgatcgaaaaattatataaagtgaagaagttggaaaagaacaatgctcttcgactgcttagttggaaagctttcagaacagaagaagttgatacaagttatttgaatgtaatggatcgtgtactagcttatgcttctggccatccattggctttggaagtaatcggttcgaagttgtttagaaaaagtgtaaaggaatgggaatctgccatcaaacagtatgagaaaattcctaacaatcaaatccttgaggtgcttaaaataagttttgatgctttagaggaagtagagaagagtgtttttcttgacatttcttgttgcttcaaagcatatgcattgtcagaggtggaagatatacttcgtgctcattatggtgattgcatgaaatatcatattggggtgttggttgaaaagtctctgataaaatatggttataattctgtagttacaatgcatgacttgatagaagacatgggtaaagaaattgtccggcagaaatctccaaataagccagggaagcgcagtagattatggtcaccagaagagataattaaagttttggaagacaacttgggaagtggagaaattgaaatcatatgtttaaattcctccttacctgacaaagaagaaatagtggaatggaacagaaaggtcttcaaaaagatgaaaaacctcaaaacacttatcattaaaaatggtaatttttcggaaggtcctgaatatcttccgaatagtttaagagtactggaatggttgaaatatccttcacaagggctaccgccagattttcgttcaaagcaactttccttatgcaaattaccttcaagttgttttgggtcactcggattgactgagttttcaaagaagttcatgaatatgactcttctgaattttgacgaatgtgaaggtttaacacagatacctgatctatctgggctgccaaatttagaaagattttcattcaagaattgtaagagtttaattacaatccatgactccattggcttcctaggtaagcttaactccttgaatgctgttggttgcagcaagcttaggagttttccccccctcaaattgacttctctggaaaatcttgaactttcatattgttacagtcttgagagcttcccagaaatattaggaaaaatgggacagataacagaacttgtcttggaggactgtcacataaaggaattgccaatttcatttcaaaatctcaccgagcttcaaacattacagttgcgttcgtgtccaaggttaaggttacctagtagtatagtcatgatgccaaaactggccaacattattgcttgggaatctaaagggtggctatttccaaaacaggtcgagggggaagagaaaataggctcaatggtgtcttcaaacgtagattgtctttatctctcagggtgcaaactctcagatcattttttcccaataattcttgaatggtttgctaatgtaaaagatttaaacctatcaaggaataatttcacggttcttcctgaatgcatcgccaattgtcacttgttatgcaagcttactttagatgcctgtcatagtcttcgagagattagagggattccaccaaacatacgacagttatcagcaagaaattgtaaatccttcacttcttcttgcggaagaactttactgaatcagaaactgcatgaggctggaaacaccatgttttcgttttcaggagcaaggtttccagaatggttcgatcaccacagttggggaccatcttgttctttctgggttggcaaaaaattcccttccattgctctttgtattgctattggaccaactcatttggaacacgttgaaattgttggacctatcatgatcatcaacagcattgaatgttcattggatgaggaggagaatccttatttatatatgctccctcatcacacacatattttcgatttgcaacatatagttttttcagattatctagacagatttgtttcagaaaatgaatggaaccacgtggagattacatactcagtcgagcagagattcgaggaaaaagacaaacatgcggtgacccaaatctctatagaaaatggaatctatgtgttcaaacagaatagcagcatggaggatattcaattcactgatccccacaaaaagagaagattagatgttgatccagagttgtaa

SEQ ID NO: 12 - protein, 1066 aa, Vigna angularis
MAVPSFSSSFTYDVFLSFRGEDTRYSFTGNLYRALRDRGIHTFIDDEKLPKGDEITSALEKAIEGSRIFIIVFSRNYASSSFCLNELAYILPYANRNGLLVLPLFYDVVPSHVRHHTGSFGEALDTHENRFKATSQGFELNMEKLNKWKMALRGTANLSGYHFKHGEEYEYEFIKRIVDLVSNKINRAPLHVADYPVGLETRVLEVKLLLDIGSDDGVHMVGIHGLGGVGKTTLALAVYNSIADHFEGLCFLENVRENSNKHGLQHLQRILLSQMIGENNVNITSARQGISMMEHRLRQKKILLILDDVDKHEQLQAIVGRPDWFGPGSRVIITTRDKHLLSCHLIEKLYKVKKLEKNNALRLLSWKAFRTEEVDTSYLNVMDRVLAYASGHPLALEVIGSKLFRKSVKEWESAIKQYEKIPNNQILEVLKISFDALEEVEKSVFLDISCCFKAYALSEVEDILRAHYGDCMKYHIGVLVEKSLIKYGYNSVVTMHDLIEDMGKEIVRQKSPNKPGKRSRLWSPEEIIKVLEDNLGSGEIEIICLNSSLPDKEEIVEWNRKVFKKMKNLKTLIIKNGNFSEGPEYLPNSLRVLEWLKYPSQGLPPDFRSKQLSLCKLPSSCFGSLGLTEFSKKFMNMTLLNFDECEGLTQIPDLSGLPNLERFSFKNCKSLITIHDSIGFLGKLNSLNAVGCSKLRSFPPLKLTSLENLELSYCYSLESFPEILGKMGQITELVLEDCHIKELPISFQNLTELQTLQLRSCPRLRLPSSIVMMPKLANIIAWESKGWLFPKQVEGEEKIGSMVSSNVDCLYLSGCKLSDHFFPIILEWFANVKDLNLSRNNFTVLPECIANCHLLCKLTLDACHSLREIRGIPPNIRQLSARNCKSFTSSCGRTLLNQKLHEAGNTMFSFSGARFPEWFDHHSWGPSCSFWVGKKFPSIALCIAIGPTHLEHVEIVGPIMIINSIECSLDEEENPYLYMLPHHTHIFDLQHIVFSDYLDRFVSENEWNHVEITYSVEQRFEEKDKHAVTQISIENGIYVFKQNSSMEDIQFTDPHKKRRLDVDPEL

SEQ ID NO: 13 - DNA, 3198 bp, Lablab purpureus
  source 1..3198: mol_type = other DNA
atggctgagccatcattctcctcttccttcacctatgacgtcttcctcagcttcagaggagaagatactcgttacagcttcaccggcaatctctacagagcccttcgtgacagcggaatctgcaccttcatcgacgacgaggagctccgcaaaggagacgagatcacctctgcgctcgagaaggcaatcgaaggctccagaatcttcatcatcgtcctctctcacaactacgcctcttcctctttctgcttgaacgagctcgcctacatccttccctacgctaagagaaagggtttgctggttttgcctctcttctacgatgtcgttccttcccacgtgcgccaccacacaggtagctttggagaagcattggatactcatgagaagaggttcaaaggtacgagtgagggttttgagcttaacatggagaagctcaacgaatggaagatggctctacgtcatgtagctaacttctctggctatcatttcaaacatggggaggagtatgaatatgagtttatcaagaagatagttgaattggtctctaacaagattaaccgtgctcctttacatgttgcggattatccagttggactagagtcacgagtgctagaaataaagttgcttctagatgtaggatctgatgatggtgtccacatggttgggatctatggactcggtggagttggtaaaaccacacttgctgttgcagtttataattccatcgctgaccattttgaaggcttgtgtttccttgaaaatgtgagagagaattcaaacaaacacggcctacatcatcttcagagccttcttctttctcaaatggttggagaaaagaatatcaacatatctagtgtgaaacaaggaatttcaattatgcagcacaggctaaggcagaagaagattcttctgattctagatgatgttgacaagcttgaacagttacagtcaattgttggaagacctgattggtttggtcctggaagtagagtcatcatcacaactagggacaaacaattgttgtcatgtcacttggttgaaaaaacatatgaagtgaagaagttgaaaaagaaggatgcttttcgactgctaagttggaaaggttttagaacagaagaagttgatacaagttatgtgaatgtcatggatcgtgtactagcttatgcttctggccatccattggctttggaagtaataggttccaagttgtttagaaaaagtataaaagaatgggaatctgccatcaaccagtatgagaaaattcctaacaatcagatcctggagatacttaaaataagttttgattctttagaggaagtagagaagagtgtttttcttgatatttcatgttgcttcaaaggatatgcattgtcagaggtggaagatatacttcgtgctcattatggtgattgcatgaaatatcatattggagtgttggttgaaaaatctctcataaaatataggtggggttgtatagttacattgcatgacttgatagaagacatgggtaaagaaattgtccgacagaaatcaccacaaaagccagggaagcgcagtagattatggtcaccagaagatataattcaagttttggaagacaactcgggaagtggagaaattgaaatcatatgcttgaactcctccttacttgacaaagaagaaacaatagaatggaacagaaaggccttcaagaagatgaaaaacctcaaaacacttatcattaaaaatggtaatttttcggaaggtcctaaatatcttccaaatagtttaagagtactggaatggttgaaatatccttcacaagggttacccccagattttcgttcaaagaaacttgtcatttgcaaattaccctcaagttgttttgggacactcgaactggctgagttatcaaagaagttcatgaatatgactgttttgaattttgacgaatgtgaaggtttaacacagatacctgatgtatctgggctgccaaatttagaaaacatttcattcaagaattgtaagagtttaattacaattcatgactccattgggttcctagataagcttaactccttgaatgctgttggttgcagcaggcttaggagttttccacctctcaaattgacttctctggaaaatcttgaactttcatattgttacagtcttgagtgcttcccagaaatattaggaaaaatggaaaagatgacagaacttgtgttggaggactgtgacatcaaggaaattccattttcatttaaaaatctcactgagcttcaaacattagatttgcgtttctgtcgaatgttaaggttaccaactagcattgtcatgatgccaaaactgtctgagattatcgcttgggaagctgaaggatgcctatttccaaaacaggttgagggtgaagagaaagtaagctcaatggtgtcttcaaatgtagattctcttcgtctctcagggtgcaaactctcggatgattttttcccaacaactcttgcatggttttctaatgtaaaagatttgaacctctcaaggaataatttcaccgttcttcctgaatgcatcagcaattgtcattttttatataagcttactttagattactgtcatagtcttcgagagattagagggcttccgccaaacatacaacatttatctgcaataaattgtacatccttcacttcttcttgcagaagtactttactgaatcagaaactgcatgaggctggaaacaccatgttttcgttttcaggagcaatgtttccagaatggttcgagaaccacagtcggggaccatcttattctttctgggttgggaacaaattccctgccattgctctttgtattgctattggaccaactcatgtggaatatgttcaaattgttggacctatcatgatcatcaatggcattgaatgttcacttcctggggtcgatccctatttaaagatgctccctgatcacacatatcttttcgatctgcagcagatagtttttccggattatctagacagatttgtttcagaaaatgaatggaaccacgtggagattacatacgcagtcgagaagagattcagtaaaaaagacaaacatgtggtgatcccggtctctatagaaaatggaatctatgtgtcaaaacagaggagcagcatggagaatgttcgattcactgatccccggaaaaaaagaagattagatgttgattcggagttgtaa

SEQ ID NO: 14 - protein, 1065 aa, Lablab purpureus
MAEPSFSSSFTYDVFLSFRGEDTRYSFTGNLYRALRDSGICTFIDDEELRKGDEITSALEKAIEGSRIFIIVLSHNYASSSFCLNELAYILPYAKRKGLLVLPLFYDVVPSHVRHHTGSFGEALDTHEKRFKGTSEGFELNMEKLNEWKMALRHVANFSGYHFKHGEEYEYEFIKKIVELVSNKINRAPLHVADYPVGLESRVLEIKLLLDVGSDDGVHMVGIYGLGGVGKTTLAVAVYNSIADHFEGLCFLENVRENSNKHGLHHLQSLLLSQMVGEKNINISSVKQGISIMQHRLRQKKILLILDDVDKLEQLQSIVGRPDWFGPGSRVIITTRDKQLLSCHLVEKTYEVKKLKKKDAFRLLSWKGFRTEEVDTSYVNVMDRVLAYASGHPLALEVIGSKLFRKSIKEWESAINQYEKIPNNQILEILKISFDSLEEVEKSVFLDISCCFKGYALSEVEDILRAHYGDCMKYHIGVLVEKSLIKYRWGCIVTLHDLIEDMGKEIVRQKSPQKPGKRSRLWSPEDIIQVLEDNSGSGEIEIICLNSSLLDKEETIEWNRKAFKKMKNLKTLIIKNGNFSEGPKYLPNSLRVLEWLKYPSQGLPPDFRSKKLVICKLPSSCFGTLELAELSKKFMNMTVLNFDECEGLTQIPDVSGLPNLENISFKNCKSLITIHDSIGFLDKLNSLNAVGCSRLRSFPPLKLTSLENLELSYCYSLECFPEILGKMEKMTELVLEDCDIKEIPFSFKNLTELQTLDLRFCRMLRLPTSIVMMPKLSEIIAWEAEGCLFPKQVEGEEKVSSMVSSNVDSLRLSGCKLSDDFFPTTLAWFSNVKDLNLSRNNFTVLPECISNCHFLYKLTLDYCHSLREIRGLPPNIQHLSAINCTSFTSSCRSTLLNQKLHEAGNTMFSFSGAMFPEWFENHSRGPSYSFWVGNKFPAIALCIAIGPTHVEYVQIVGPIMIINGIECSLPGVDPYLKMLPDHTYLFDLQQIVFPDYLDRFVSENEWNHVEITYAVEKRFSKKDKHVVIPVSIENGIYVSKQRSSMENVRFTDPRKKRRLDVDSEL

SEQ ID NO: 15 - DNA, 3195 bp, Mucuna pruriens
  source 1..3195: mol_type = other DNA
atgggtttggcatcatattccttgtccttcagctacgatgtgttcctcagtttcagaggctcagacacgcgccatggtttcactggtaatctctacaaagccctgcgtgacaggggaattcacaccttcatcgacgacgaggacctccagagaggggaccaaatcacgttggcacttgagaaggcaattgaatgctctagaattttcatcattgtgctctctcacaactatgcttcttcttcgttttgcttgaacgaactcgcttacatcctcgactgcattaagactaaaggtttgttggttttgccaattttttatgacgtgagacaccacattggtagtttcggagaagcgttggctaatcatgaaaagaagttcaatgctaagagggatggcttcgaccttaatgttgagaagctgaataaatggaaaatgtctctacatcaagcagctaacttatctggctatcatttcaaacacggggatggatacgaacacgagtttattaagaggatagttgatttagtctctagcaaggttaaccgtgctcctttacatgttgcggattacccgattggacttgagtcacgagtgctacaagtaaagtcacttctggatgttggatctgatgatggtgtccacatggtagggatccatggactcggtggaattggtaaaaccacactggctgttgcagtttataattccattgctgaccactttgaaggtttatgttttcttgaaaacgtgagagaaaactcacacaaacatgggttacaacacgtccagagcatccttctttctgaattggttggtgaaaagaatatcaacttagcaagtgtgaaacaaggaatttcaatgatacagcataggctacgccaaaagaagattcttttgattcttgatgacgttgacaaacacgagcagttacaagcaattgttggaaaacctgattggttgggtcctggcagcagagtcatcatcacgactcgggacaaacaattgttggcatgtcatgaagttaaaagaacatacgaagtgaagaagttgaacaaaaacgattctcttcaactgcttagttggaaagcttttagaacagaaaaagttgatacaagttatgtggatgtcatgaatcgtgtagtaacttatgcttctggccttccattggctttggaagtaataggttccaacttgtttggaaaaagtatacaagaatgggaatctgctataaatcagtataaaaaaattcctaataatcaaatcctaaaggtgcttaaaataagttttgatgctttagaggaagaagagaagagtgtttttcttgacattgcttgttgcttcaaaggatatgaattgacagaggttgaagatatacttggtgctcattatggtgattgcatgaaatatcatattggggtgttggttaaaaaatctctcataaaacttagttggcatggttcagttacattgcataacttgatagaggacatgggtaaggaaattgtccgacagaaatcaccaaaagagccagggaagcgtagtagattatggtcatcggaagatataattcaagttttggaagagaactcgggaactagtcaaattgaaatcatatgtttggttttttcctttcatgacaaagaacaaatagtagaatggaatcgaaaggccttcaagaagatgaaaaacctcaaaacacttattattaaaaatggtaatttttccaaaggtcccaaacatcttccaaatagtttaagagtactggaatggtgtaaatatcctgcacaagggttaccacctgattttcgttcaaagaaactagccgtatgcaagttacctgaaagttgttttgggtcactcgagttggccgacttatcaaaggcaagtaagttcgtgaatatcaatgtcttgaattttgacgaatgtgaaggtttaacccaaatacctgacatatctgggctgtcaaatttagaaaaaatttcattccgaaatagtgagaatttaattacaatccatgagtccttagggttcctaggtaagcttaaattcttggatgcctttggttgcagcaagcttaggagttttccacccctcatgttgacttctctggaaaaacttgaactttcatattgttccagtcttgagagtttcccagaaatattaggaaaaatggaaaatataagtgaacttgtgttagaggcctctggaattaaagaattgccattttcatttcaaaatctcagtgggcttgaaacattacaattgcgtcattgtggaatgttaaggttaccaagtagcattgtcatgatgccaagattggctgagattattgcttgggaatggaaaggatggctattgccagaacaggttgaggatgaagaaaaagtaaactcaatggtggtgccttcaaatgtagattgtcttcgtctctccggctgcaacctgtccgataatcttttatcaataggtctcacatggtttgctaatgtaaaagatttagacctatcgaggaataatttcacagttcttcctgaatgcatcagttattgtcattttttatggaagcttcatttagattattgtcattttcttcgagagattagagggattccgccaaacatacaacatttctcagcaagagtttgtaaatccttgagttcgtcttctagaagcacattactgaatcagaaagtgcatgaggctggaaacaccatgttttggttgtcaggagcaatatttccagagtggttggagcagcacaggcagggaccatctaattgtttctggtttcgtaacaagttccctgccattgctctctgtattgctattggaccaactcattacaaatatgttgaaattgttggacctattgtgatcatcaatggcattgaatgtttacttgacacggaccatgatccctatttatggatgatgactgatcacacttatctttttgatctgcaaaagacaaaagttgcagatattctgaacaaactagctatagaaaatgaatggaagcatgtggagattacatacgaagttacgcagagattcattgcaaaagaaaagcaggtggagatcccagtctttattgaaagcggtatatatgtgttcaaagagaggaatagcatggaggatattcaatttgttgatccttataaaaagagaagattagatgatgaccaaagagcatag

SEQ ID NO: 16 - protein, 1064 aa, Mucuna pruriens
MGLASYSLSFSYDVFLSFRGSDTRHGFTGNLYKALRDRGIHTFIDDEDLQRGDQITLALEKAIECSRIFIIVLSHNYASSSFCLNELAYILDCIKTKGLLVLPIFYDVRHHIGSFGEALANHEKKFNAKRDGFDLNVEKLNKWKMSLHQAANLSGYHFKHGDGYEHEFIKRIVDLVSSKVNRAPLHVADYPIGLESRVLQVKSLLDVGSDDGVHMVGIHGLGGIGKTTLAVAVYNSIADHFEGLCFLENVRENSHKHGLQHVQSILLSELVGEKNINLASVKQGISMIQHRLRQKKILLILDDVDKHEQLQAIVGKPDWLGPGSRVIITTRDKQLLACHEVKRTYEVKKLNKNDSLQLLSWKAFRTEKVDTSYVDVMNRVVTYASGLPLALEVIGSNLFGKSIQEWESAINQYKKIPNNQILKVLKISFDALEEEEKSVFLDIACCFKGYELTEVEDILGAHYGDCMKYHIGVLVKKSLIKLSWHGSVTLHNLIEDMGKEIVRQKSPKEPGKRSRLWSSEDIIQVLEENSGTSQIEIICLVFSFHDKEQIVEWNRKAFKKMKNLKTLIIKNGNFSKGPKHLPNSLRVLEWCKYPAQGLPPDFRSKKLAVCKLPESCFGSLELADLSKASKFVNINVLNFDECEGLTQIPDISGLSNLEKISFRNSENLITIHESLGFLGKLKFLDAFGCSKLRSFPPLMLTSLEKLELSYCSSLESFPEILGKMENISELVLEASGIKELPFSFQNLSGLETLQLRHCGMLRLPSSIVMMPRLAEIIAWEWKGWLLPEQVEDEEKVNSMVVPSNVDCLRLSGCNLSDNLLSIGLTWFANVKDLDLSRNNFTVLPECISYCHFLWKLHLDYCHFLREIRGIPPNIQHFSARVCKSLSSSSRSTLLNQKVHEAGNTMFWLSGAIFPEWLEQHRQGPSNCFWFRNKFPAIALCIAIGPTHYKYVEIVGPIVIINGIECLLDTDHDPYLWMMTDHTYLFDLQKTKVADILNKLAIENEWKHVEITYEVTQRFIAKEKQVEIPVFIESGIYVFKERNSMEDIQFVDPYKKRRLDDDQRA

SEQ ID NO: 17 - DNA, 3201 bp, Cajanus cajan
  source 1..3201: mol_type = other DNA
atggctgtggcatcatcttccttgatctatgattatgaatatgatgtgttcctgagcttcagaggagaagatacccgttacggtttcaccggaaatctctacaacgccctccgtgacaacggaattcacacattcatcgacgacgaccaactccaaaaaggggacgagatcacatcagcacttgagaaggccatcgaacactcgagaattttcatcatcgtcctctctctcaactacgcttcttcctctttttgcttaaacgagctcacttacatccttcactgcactaagagaaaatctttgttggttttgcctctcttttataacatcgatccttctgaccttcgtcaccacagaggtagcttcggagaagcgttgaccaatcatgaaaacaatttcaaagctaagaaggaggggttggagcataacatggacaagctccacaaatggaagatggctcttcatcaagcagccagcttgtctggctatcatttcaaacaaggggatggatatgaatacgagtttattaaaaagatagttaaattggtatctagcaagattaaccgtgctcctttgcatgttgcggattacacggttggacttgagtctcgagtgctggaagtaaagttgcttctaaatgttgggtctgatgactgtgtccacatggttggaatccatggtattggtggaattggtaaaaccacacttgctgttgaagtttataattccattgctgaccattttgaaggtttatgctttcttgaaaacgtcagagaaaattcaaataaacatggtttacaacatctccagagcatccttctatctgaaatggttggagaaaagaatatcaacttagcaagtgttagacaaggaatttcaatgatacaacataggttacggcaaaagaaggttcttttgattgtagatgatgttgataagcacgagcagttacaggctattgttggagaacctaattggtttggtcccggcagcagagtcatcatcactactcgagacaaacaactgctggcatgtcaccaggttaaaacaaactatgaagttgaaaagttgaataagagcgatgctctacaactgctcagttggaaagcttttaaaactgacaaagttgatacaagttatgtaaatgtcatgaatcgtgtagtaacttatgcttctggccttccattggctttggaagtaataggttccaacttattcggaaaaactgtaggagaatgggaatctgctataaatcaatatgaaaaaattcctaataatcaaatctttaagatacttaaaaaaagttttgatgctttagaagaacaagagaagagcgtttttcttgacattgcttgttgcttcaaaggatttcgattgaaagaggtcgaagatatactttgtgctcactatggggattgcatgaaatatcatattagggtgttggttgaaaaatctctcataaaacagaactggtatggtgcagttacattgcatgacttgatagaagacatgggtaaagaaattgtccgacagaaatcaccaaaagagccagggaagcgcagtagattatggtctccggatgatataattcaagttttggaagatgactcgggtactggtaaaattgaaatcatatgtttggatttctccctacttgacaaagaagaaatagtagaatggaatcgaaaggctttcaagaagatgaaaaatctcaaaacacttattattaaaaatggtcatttttccaaaggtcccaaacatcttccgaatagtttaagagtactggaatggttgaaatatccttcaaaagggttaccaactgatttttgttcaaagaaactctccatatgcaagttacctaaaagctgttttgggtcactggagttggctgacttatcaaagaagttcacgaatatgactgttttgaattttgaggaatgccaaggattaacacaaataccagatgtatctgggctgccaaatttagaaaatatttcattccgaaatagtgacaatttagctacaatccatgactccattgggttccttggtaagcttaaaattttgaatgcttttggttgcaagaaacttaggagttttccaccgctcaagttggaatctcttgaaaaacttgaactttcatattgttccagtctagagagtttcccagaaatattaggaaaaatggaaaacataacagaacttgtgttggaggcttctgccataaagaattttccagtttcatttcaaaatctcactgggcttcaagaattactgttgcgtcactgtggagtgttaacgttaccaagtagcattgtcatgatgccaaaattggttcagatcattgcttgggaatggaaagggtggctattcccaaaacaggttgagggtgaagaaaaagttcgttcaatgatgtcttcaaacgtaaattgtcttcgtctctcaggctgtaacctgtccgatgattttttttcaataggtctttcatggtttgctaatgtaaaagaattagacctatcaagaaataatttcaaagttcttcctgaatgcatcagtgattgtcaattcctactggagcttaacttagattattgttattgtcttcaagaaattagagggattccgccaagcatagaacttttctctgcaagagaatgtaaatccttgacctcttctggtagaagcacattagtgaatcagaagctgcatgaggttggaaacaccttgtttaggttttcaggagcaaggtttccagagtggttcgaccaccaaagccggggatcatctaattctttttggtttcgccacaagttccctgccatttgtctttgtattgctattggaccaactcattacgaatattttaaaattgttggacctaccttgaccatcaatggcattgaatgtttacctgacgatgaggattttgactatttttggatggagcctgatcacacacatcttttcgatctgcaaaagatgcatctcagagataatctagacaaatatgttgtagaaaatgaatggaaccatgtggagattacttaccatccgacaatcggggaaagacagaaggttgtggagatcccagtctttactgaaactggtatctatgtgtacaaacagaggagtagcatggtggatattcaatttactgatccttataaaaggagaagattggatgatgactcaggatcataa

SEQ ID NO: 18 - protein, 1066 aa, Cajanus cajan
MAVASSSLIYDYEYDVFLSFRGEDTRYGFTGNLYNALRDNGIHTFIDDDQLQKGDEITSALEKAIEHSRIFIIVLSLNYASSSFCLNELTYILHCTKRKSLLVLPLFYNIDPSDLRHHRGSFGEALTNHENNFKAKKEGLEHNMDKLHKWKMALHQAASLSGYHFKQGDGYEYEFIKKIVKLVSSKINRAPLHVADYTVGLESRVLEVKLLLNVGSDDCVHMVGIHGIGGIGKTTLAVEVYNSIADHFEGLCFLENVRENSNKHGLQHLQSILLSEMVGEKNINLASVRQGISMIQHRLRQKKVLLIVDDVDKHEQLQAIVGEPNWFGPGSRVIITTRDKQLLACHQVKTNYEVEKLNKSDALQLLSWKAFKTDKVDTSYVNVMNRVVTYASGLPLALEVIGSNLFGKTVGEWESAINQYEKIPNNQIFKILKKSFDALEEQEKSVFLDIACCFKGFRLKEVEDILCAHYGDCMKYHIRVLVEKSLIKQNWYGAVTLHDLIEDMGKEIVRQKSPKEPGKRSRLWSPDDIIQVLEDDSGTGKIEIICLDFSLLDKEEIVEWNRKAFKKMKNLKTLIIKNGHFSKGPKHLPNSLRVLEWLKYPSKGLPTDFCSKKLSICKLPKSCFGSLELADLSKKFTNMTVLNFEECQGLTQIPDVSGLPNLENISFRNSDNLATIHDSIGFLGKLKILNAFGCKKLRSFPPLKLESLEKLELSYCSSLESFPEILGKMENITELVLEASAIKNFPVSFQNLTGLQELLLRHCGVLTLPSSIVMMPKLVQIIAWEWKGWLFPKQVEGEEKVRSMMSSNVNCLRLSGCNLSDDFFSIGLSWFANVKELDLSRNNFKVLPECISDCQFLLELNLDYCYCLQEIRGIPPSIELFSARECKSLTSSGRSTLVNQKLHEVGNTLFRFSGARFPEWFDHQSRGSSNSFWFRHKFPAICLCIAIGPTHYEYFKIVGPTLTINGIECLPDDEDFDYFWMEPDHTHLFDLQKMHLRDNLDKYVVENEWNHVEITYHPTIGERQKVVEIPVFTETGIYVYKQRSSMVDIQFTDPYKRRRLDDDSGS

SEQ ID NO: 19 - DNA, 3177 bp, Abrus precatorius
  source 1..3177: mol_type = other DNA
atggctgttagatcatgttcttcttccttcacctatgcattcacctatgatgttttccttagctttagaggctcagatacacgtcatggttttgttggcaatctatttaaagctcttcaagacaagggaattcacaccttcattgatgatgaggggcttcaaagaggagaggaaatatcaccagcacttgtcaaggcaattgaagagtcaagaattgccatcgctgtgctctctaataattatgcatattcttcattttgcttagatgaacttgcccacatccttgagtgtgttaagagaaaagatacattggttttgccccttttttatgatgttgatccctctcatgtgagataccaaagaggtagttatggagaagcattggcaagtcatggagaaaggttcaagcataacatggagaagttgcagaaatggaagatggctttgcatcatgtagctaacttgtctggctaccatttcaaacacggagatggttatgaatacgagtttattgggaggattgttgagttggtttccaacaagattaatcgtgctcctttacatgttgcggattatcctgttggactagagtcacaagtgctggaagtaaggaaacttttggatgttggatctgatgatggtgttctaatgatagggattcatggaattggtgggataggaaaaacaacacttgctctggcagtttacaatttgattgctgaccattttgatggtttgtgttttctagaaaatgtgagagaaaattcagacaaacatgggttacaacatctccaaagcatcctactttcagaaatacttggagagacgaaaattaagttagcgagtgtcaaacaaggaatttcaatactacaacacaggctccagcgacagaaggttctcttgattctagatgatgtcgacaagcacgagcaattaaatgccattgttggaaaacctgattggtttggtcctggcagtagagtcatcatcacgacccgggacaaacatctactagcatgtcatgaagttaaaagtacacatgaggtgaagaaattgaataagaacgatgctcttcaattacttagttggaaagcatttaaatcagaaaaagttgatccaagttatgtggatgtcatgaattgtgcagtagcttatgcttctggtcatccattggctttggaagtaatcggttccaacttgtttggtaaaagtatagaacaatggaaatctgctatcaatcaatataaaagaattccaaattgtcatatcctaaagatacttaaagtaagctttgatgctttagaggaagaagagaagagtgtttttcttgacattgcctgttgctttaaaggatataacttagaagaagttgaagatatacttcatgctcattatggtgactccatgaaatatcatattgggttgctggttgaaaaatctctcataaagcttacttgggctggtggggtgacattgcatgacttgatagaggacatgggtaaagagattgtccgacagaaatcaccgaaagatcctgggaagcgcagtagattatggttaccggaagatataattcatgttttcgaacacgactcgggaactggtgaaattgaaatcatacgtctggcttcctccttacttgacaaagaagaaataattaaatggaatagaaaggccttcaagaagatgaaaaacctcaaaacacttataattaaaaatggtcatttttccaaaggtcccaaacatcttccgaatagtttaagagtactggaatgggcgaaatatccttcacaagggtttccagctaatttttgttcaaagaaactgagcatatgcaagttacctaaaagttgtttctcactagagttggctgacttatcagagaagttcatgaacatgagtgttttgaattttgacgagtgtgaaggtttaacacaaataccagatgtatcggggctccaaaatttagaaaaattttcattcaaaaattgtgagaatttaattacaattcatgattccattggattcctacataagcttaaattcttgaatgctataggttgcagaaagctcaggagttttccacccctcaaactgacctctcttgagaaacttgaactttcatattgttccaatcttgagtgttttccagaaatattagggaaaatggaaaatataacagaacttgtgttggaggcctctgccataaaagaattgccattttcatttcaaaatctcactgggcttcaaatattacagttgcgtctaggaggaatgattaggttacctagcagcattgtcatgatgccaaaattgactgagattattgcttgggattggaaagggttgctatggccaagacaggttgagggtgaagaaaaagtaagctcaatggtgtcttcaaatgtagattgtctttgtctctcaggctgcaacctgtctgatcaatttttaccagtagctctctcatggtttgttaatgtaaaagatttagacctatcaaggaacaagttcacagttcttcctgaatgcatcagtgaatgccattttttatggaagcttattttggattattgcaattgtcttcgagaaattagagggatgccaccaaacatagaacatttctctgcaagaaactgtaaatccttgacttcttgtagaagcacattactgaatcagaaactgcatgaagctggaaacaccatgttttggttgtcaggaacatggtttccagagtggttcgagcaccacagcaagggactgtctaattctttctggtttcgtgataagtttcctgccatagctctttgtactgctattggaccaacgcgtgaacagattacaattgttggacctattgtgatcatcaatggcattgaatgttcggttgatgatgaggatgattcctatttatggatggagactgatcatacatatctttttgatctgcaaaagataaatttcgcagataatttggacaaagaacttgtagaaaatgaatggaaccatgtggagattacatactcagtcatatccaatgaaaaggaaaaacatgtggagattccagtctttatagaaagtggaatctatatattcaaacagaggagtagaatggaggatattcgattcactgatccttataaaaggagaaaattagatgatggtttggaatcataa

SEQ ID NO: 20 - protein, 1057 aa, Abrus precatorius
MAVRSCSSSFTYAFTYDVFLSFRGSDTRHGFVGNLFKALQDKGIHTFIDDEGLQRGEEISPALVKAIEESRIAIAVLSNNYAYSSFCLDELAHILECVKRKDTLVLPLFYDVDPSHVRYQRGSYGEALASHGERFKHNMEKLQKWKMALHHVANLSGYHFKHGDGYEYEFIGRIVELVSNKINRAPLHVADYPVGLESQVLEVRKLLDVGSDDGVLMIGIHGIGGIGKTTLALAVYNLIADHFDGLCFLENVRENSDKHGLQHLQSILLSEILGETKIKLASVKQGISILQHRLQRQKVLLILDDVDKHEQLNAIVGKPDWFGPGSRVIITTRDKHLLACHEVKSTHEVKKLNKNDAQLLSWKAFKSEKVDPSYVDVMNCAVAYASGHPLALEVIGSNLFGKSIEQWKSAINQYKRIPNCHILKILKVSFDALEEEEKSVFLDIACCFKGYNLEEVEDILHAHYGDSMKYHIGLLVEKSLIKLTWAGGVTLHDLIEDMGKEIVRQKSPKDPGKRSRLWLPEDIIHVFEHDSGTGEIEIIRLASSLLDKEEIIKWNRKAFKKMKNLKTLIIKNGHFSKGPKHLPNSLRVLEWAKYPSQGFPANFCSKKLSICKLPKSCFSLELADLSEKFMNMSVLNFDECEGLTQIPDVSGLQNLEKFSFKNCENLITIHDSIGFLHKLKFLNAIGCRKLRSFPPLKLTSLEKLELSYCSNLECFPEILGKMENITELVLEASAIKELPFSFQNLTGLQILQLRLGGMIRLPSSIVMMPKLTEIIAWDWKGLLWPRQVEGEEKVSSMVSSNVDCLCLSGCNLSDQFLPVALSWFVNVKDLDLSRNKFTVLPECISECHFLWKLILDYCNCLREIRGMPPNIEHFSARNCKSLTSCRSTLLNQKLHEAGNTMFWLSGTWFPEWFEHHSKGLSNSFWFRDKFPAIALCTAIGPTREQITIVGPIVIINGIECSVDDEDDSYLWMETDHTYLFDLQKINFADNLDKELVENEWNHVEITYSVISNEKEKHVEIPVFIESGIYIFKQRSRMEDIRFTDPYKRRKLDDGLES

SEQ ID NO: 21 - DNA, 3198 bp, Vigna unguiculata
  source 1..3198: mol_type = other DNA
atggctgtgccatcattctccttttccttcacctatgacgttttcctgagcttcagaggagaagacactcgttacggtttcaccggaaatctttacagagcccttcgtgacagaggaattcacaccttcatcgacgacgaggagcttcgcaaaggagacgaaatcacctctgcacttgagaaggcaatcgaaggttctagaattttcatcatcgttttctctctcaactacgcctcttcctctttttgcttgaacgagctcgcctacatccttccccacgctaagagaaatgctttgctggttttgccactcttctacgacgtcgttccttcccacgtgcgacaccacacgggtagctttggagaagcattggatgctcatgaaaagaggttcagaggtatgagtcagggttttgagcttaacattgagaagctcaacaaatggaagatggctctgcgtcgagcagctaacttgtctggctatcatttcaaacatggggaggaatatgaataccagtttatcgagaagatagttaaattggtctctaacaagattaaccgtgttcctttacacgttgcggattatcaggtaggactagaaaagcgagtgctggaagtaaagatgcttgtagatgtaggatctgatgattgtgtccacatggtaggaatctatggactcggtggagttggtaaaaccacacttgctcttgcactttacaattccatcgctgaccattttgaaggtttgtgtttccttgaaaatgtgagagagaattcaaacaaacaaggcttacatcatcttcagagaatccttctttctgaaatggttggagaaaataatatcaacataggtagtgtgagacaaggaatttcaatgatgcagcataggctacggcagaagaagattcttttgattctagatgatgttgacaagcacgaacagttacaggcaattgttggaagccctgattggtttggtcccggcagtagagtcatcatcacaactagggacaaacaattgttgtcatgtcacttggttgaaaaattatatgaagtgaagaagttggaaaagaacgatgctcttcgattgctcagttggaaaggttttagaaccgaagaagttgatacaagttactccaatgtcatggatagtgtactagcttatgcttctggccatccattggctttggaagtaatcggttcgaagttgttcagaaaaagtgtagaagaatgggaatctgccatcaaacagtatgagaaaattcctatcagtcaaatcctggaggtgcttaaaataagttttgatgctttagaggaagtagagaagagtgtttttcttgacattgcttgttgcttcaaaggatatgcattgtcagatatagaagatattcttcgtgcccaatatggtcatagcatgaaatatcatattggggtgttggttgaaaaatctctcataaaatacaaatggaaatttggagttacaatgcatgacttgatagaagacatgggtaaagaaattgtccgacagcaatcaccaaaaaggccagggaagcgcagtagattatggtcaccagaagatataattcaagttttggaagacaactcgggaagtggagaaattgaaatcatatgtttgaattcttcattacctgacaaagaagaaataatagaatggaacagaaaggccttcaagaagatgaaaaacctcaaaacacttatcattaaaaaaggtaatttttcggaaggtcctaaatatcttccaaatagtttaagagtactggaatggttgaaatatccttcacaagggctaccgccagattttcgttcaaaggaacttgccatatgcaaattacctacaagttgttttgggtcactcgaattggctgagttatcaaagaaattcatgaatatgactcttttggattttgaagaatgtcaaggtttgacacagatacctgatgtatctgggatgccaaatttagaaaaaatttcattcaagaattgtaagagtttaaatacaatccatgactccattgggttcctaggtaagctgaactccttgaatgctgttggctgcagcaagcttaggagttttccccccctcaaattgacttctctgaaaaatcttgaactttcatattgttacggtctcgagagcttcccagaagtattaggaaaaatgggaaagataacaaaacttgtcttggaggactgtaacataaaagaattgccattttcatttcaaaatctcactgagcttcagacattacagttgcgtttctgtccaatgttacggttaccaagtagtatagtcatgatgccaaaactggccgaaattattgtttgggaatctaaaggatggctatttccaaaacaagttgaggttgaagagaaagtaagttcaatggtgtcttcaaatgtagattgtcttcttctcccacggtgcaaactctccgatgattttctcccaatagctcttgcatggtttgctaatgtaaaagagttagacctgtcatggaacaatttcacggttcttccggaatgtattagcaattgtcattttttatctaagcttactttagatggctgtcatagtcttcgagagatcagagggattccaccaaacattcaacttttactagcagtagattgtaaatccttcacttcttcttgcagaagcactttactgaatcagaaactgcatgaggctggaaacaccatgtttcggttgtcaggagcaagctttccagaatggttcgatcaccacaatcagggaccatcgtgttctttctgggttggcaacaaattcccttcaattgctctttgtattgctattggaccagctcatttggaacatgttacaattgatagacctatcataaacatcaatggcgtcaaatgttcgcttcatggggaggaaaaaccttatttaaatatgctccctcatcacacatatctttttgatttgcaacatatagttttttcagattatctagacagatttgttacagaaaatgaatggaaccacgtggagattacatactcagtcaagcagagattcaatgaaaaagacaaacatgcggtgaccccagtctctgtagaaaatggaatctatgtgttgaaacagaggagcagcatggaggatattcaattcactgatccccacaaaaagagaagattagatgttgatccagagtag

SEQ ID NO: 22 - protein, 1065 aa, Vigna unguiculata
MAVPSFSFSFTYDVFLSFRGEDTRYGFTGNLYRALRDRGIHTFIDDEELRKGDEITSALEKAIEGSRIFIIVFSLNYASSSFCLNELAYILPHAKRNALLVLPLFYDVVPSHVRHHTGSFGEALDAHEKRFRGMSQGFELNIEKLNKWKMALRRAANLSGYHFKHGEEYEYQFIEKIVKLVSNKINRVPLHVADYQVGLEKRVLEVKMLVDVGSDDCVHMVGIYGLGGVGKTTLALALYNSIADHFEGLCFLENVRENSNKQGLHHLQRILLSEMVGENNINIGSVRQGISMMQHRLRQKKILLILDDVDKHEQLQAIVGSPDWFGPGSRVIITTRDKQLLSCHLVEKLYEVKKLEKNDALRLLSWKGFRTEEVDTSYSNVMDSVLAYASGHPLALEVIGSKLFRKSVEEWESAIKQYEKIPISQILEVLKISFDALEEVEKSVFLDIACCFKGYALSDIEDILRAQYGHSMKYHIGVLVEKSLIKYKWKFGVTMHDLIEDMGKEIVRQQSPKRPGKRSRLWSPEDIIQVLEDNSGSGEIEIICLNSSLPDKEEIIEWNRKAFKKMKNLKTLIIKKGNFSEGPKYLPNSLRVLEWLKYPSQGLPPDFRSKELAICKLPTSCFGSLELAELSKKFMNMTLLDFEECQGLTQIPDVSGMPNLEKISFKNCKSLNTIHDSIGFLGKLNSLNAVGCSKLRSFPPLKLTSLKNLELSYCYGLESFPEVLGKMGKITKLVLEDCNIKELPFSFQNLTELQTLQLRFCPMLRLPSSIVMMPKLAEIIVWESKGWLFPKQVEVEEKVSSMVSSNVDCLLLPRCKLSDDFLPIALAWFANVKELDLSWNNFTVLPECISNCHFLSKLTLDGCHSLREIRGIPPNIQLLLAVDCKSFTSSCRSTLLNQKLHEAGNTMFRLSGASFPEWFDHHNQGPSCSFWVGNKFPSIALCIAIGPAHLEHVTIDRPIININGVKCSLHGEEKPYLNMLPHHTYLFDLQHIVFSDYLDRFVTENEWNHVEITYSVKQRFNEKDKHAVTPVSVENGIYVLKQRSSMEDIQFTDPHKKRRLDVDPE

SEQ ID NO: 23 - DNA, 663 bp, Phakopsora pachyrhizi
  source 1..663: mol_type = other DNA
atgcgatcaacggtgttggttggttggataaccagtggctttgtagcactggtcttacacgccaccagtgcaacccaacagaagaaaggaccagtgattgtttcaaaggtatactttgatattcaacaaggttcaaagagtttgggtaggatcgagataggtcttttcaaaggtacaccaaagactagtgagaacttcagatcactctcagttggtgagaagagatcaaaggatggaatcgatctatcttacaaaggttcaaagtttcatagagttataaaaaactttatgattcagggtggtgactttacaaacggtgatggtacaggtgggatttcaatctacggatcaaaatttccagatgagaactttaaacacaaacatactggagctggaatagtttctatggctaattctggtcctaatacaaatggatctcaattctttatctgcactgctgttacgtcttggttagatggaagacacgttgtttttggaaaagttataaaaggcttagacgttgttaccaaaattgaacaatcacctactaatggtaaagataatcctctcgaagatatcatcatttcagattcgggtgagatcaagatagaagaagatttggattcagatggaaagaagattccactgagagtagaattatga

SEQ ID NO: 24 - protein, 220 aa, Phakopsora pachyrhizi
MRSTVLVGWITSGFVALVLHATSATQQKKGPVIVSKVYFDIQQGSKSLGRIEIGLFKGTPKTSENFRSLSVGEKRSKDGIDLSYKGSKFHRVIKNFMIQGGDFTNGDGTGGISIYGSKFPDENFKHKHTGAGIVSMANSGPNTNGSQFFICTAVTSWLDGRHVVFGKVIKGLDVVTKIEQSPTNGKDNPLEDIIISDSGEIKIEEDLDSDGKKIPLRVEL

SEQ ID NO: 25 - DNA, 663 bp, Phakopsora pachyrhizi
  source 1..663: mol_type = other DNA
atgcactcgaaagtgttgatatcgtgggtcatgagtgcctttgtggcaatcgtcctacatgcagctaattcgatccaggacaatgaggagccaccaatcgttgcaaaggtgtattttgatatgaaacaaggctcaaagaatcttggtaggattgagatcggtttattcaaaaatacgcccaagactagtgaaaacttcagagcacttgcagttggtgataaaaaatcaaaagatgggattcctctggcctacaagggctctaagtttcacagagtcattaaccaatttatgattcaaggtggagattttactaaaggcgatggaactggcggactatctatctacggaccaaagtttcaagacgaaaactttataaacaaacatactggacctggaactgtttctatggcaaactctggtcctgatacgaacggctctcagttttttatctgcactgctcttacttcttggttagatggtagacacgtagtttttggaaaagttcttagaggaatggatgttgtttttcgaattgaaactttacctactgacaatggagacaggcccaaggaagatatcgttatagccgactctggtgaactcaagatcgatgatgaacttgatgcagacggaaacaaggtggctcctcgtgtagaattataa

SEQ ID NO: 26 - protein, 220 aa, Phakopsora pachyrhizi
MHSKVLISWVMSAFVAIVLHAANSIQDNEEPPIVAKVYFDMKQGSKNLGRIEIGLFKNTPKTSENFRALAVGDKKSKDGIPLAYKGSKFHRVINQFMIQGGDFTKGDGTGGLSIYGPKFQDENFINKHTGPGTVSMANSGPDTNGSQFFICTALTSWLDGRHVVFGKVLRGMDVVFRIETLPTDNGDRPKEDIVIADSGELKIDDELDADGNKVAPRVEL

SEQ ID NO: 27 - DNA, 8574 bp, synthetic construct
  misc_feature 1..8574: note = Transient expression vector with Vigna unguiculata FIT1
  source 1..8574: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatggctgtgccatcattctccttttccttcacctatgacgttttcctgagcttcagaggagaagacactcgttacggtttcaccggaaatctttacagagcccttcgtgacagaggaattcacaccttcatcgacgacgaggagcttcgcaaaggagacgaaatcacctctgcacttgagaaggcaatcgaaggttctagaattttcatcatcgttttctctctcaactacgcctcttcctctttttgcttgaacgagctcgcctacatccttccccacgctaagagaaatgctttgctggttttgccactcttctacgacgtcgttccttcccacgtgcgacaccacacgggtagctttggagaagcattggatgctcatgaaaagaggttcagaggtatgagtcagggttttgagcttaacattgagaagctcaacaaatggaagatggctctgcgtcgagcagctaacttgtctggctatcatttcaaacatggggaggaatatgaataccagtttatcgagaggatagttgaattggtatctaagaagattaaccgtgctcctttacacgttggggattatccagttggactagaggcgcgggtgctagaagtaaagttgcttctagaagtaggatctgatgatgttgtccacttggtagggatccatggactcggtggaatcggtaaaaccacacttgctcttgcactctacaattccatcgctgaccattttgaaggtttgtgtttcctcgaaaatgtgagagagaattcaaacaaacacggcctacatcatcttcaaagaatccttctttctcaagtgcttggagaaaataatatcaacataactagtgtgagacaaggaatttcaatgatgcagcataggctacggcagaagaagattctcttgattctagatgatgttgacaagcacgaacagttacaggcaattgttggaagacctgattggtttggtcccggcagtagagtcatcatcacaaccagggacaaacagttgttgtcgtgtcacttggttgaaaaattatataaagtgaagaagttggaaaagaacaatgctcttcgactgcttagttggaaaggttttagaacagaagaagttgatacaagttatttgaacgtcatggatcgtgtactagcttatgcttctggacatccattggctttggaagtaataggttcgaagttgtttagtaaaagtgtaaaagaatgggaatctgccatcaaacagtacgagaaaattcctagcaatcaaatccttgaggtacttaaagtaagttttgatgctttagaggaagtagagaagagtgtttttcttgacattgcttgttgtttcaaaggatatgcattgtcagagatagaagatattcttcgtgctcattatggtgattgcatgaaatatcatattggagtgttagttgaaaaatctctcatgaaatatggctataattctgtagttacgttgcatgacttgatagaagacatggggaaagaaattgtccgagagaaatcaccaaaaaatccagggaagcgcagtagattatggtcaccagaagatataattcaagttttggaagacaattcgggaagtggagaaattgaaatcatatgtttgaattcctccttacctgacaaagaagaaatagtaggatggaacagaaaggccttcaaaaagatgaaaaacctcaaaacacttatcattaaaaaaggtaaattttcggaaggtcctaaatatcttccaaatagtttaagagtacttgaatggttgaaatatccttcacaagggctaccaccagattttcgttcaaagaaacttgccatatgcaaattaccttcaagtagttttgggtcactcgaattggctgagttttcaaagaagttcatgaatatgactcttctgaattttgatgaatgtgaaggtttaacacatatacctgatgtgtctgggctgccaaatttagaaaaagtttcattcaagaattgtaagagtttagttacaatccatgactccttcgggttcctaggtaagcttaactccttgagtgctgttggttgcagcaagcttaggagttttcctcccctcaaattgacttctctggaaaatcttgaactttcatattgtcacagtctcgagagcttcccagaaatattagaaaaaatgggaaagataacagaacttgtcttggaggattgtcacataaaagaattaccattttcatttcataatctcaccgagcttcaaacattacagttgcgttggtgtccaatattaaggttaccaagtagtattgtcatgatgccaaaactggcccagattattgcttgggaatctaaaggatggctatttccaaaacaggttgatggggaagaaaaaggaagctcaatggtgtcttcaaatgtagattgtcttgttctctcagggtgcaaactctcagatgactttttcccagtaattcctgaatggttttctaatgtaaaagatttagacctgtcaaggaataatttcacggttcttcctgaatgcatcagcaattgtcattctttatgcaagcttactttagatagctgtcatagtcttcaagagattcgagggattccacctaacatacgacatttatcagcaagaaattgtaaatccttcacttcctcttgcagaagcactttactgaatcagaaactgcatgaggctgggaacaccatgttttggttgtcaggagcaaagtttccagaatggttcgatcaccacggtcggggaccatcttgttctttctgggttggcaacaaattcccttcaattgctctttgtattgctattggacaaactcatatcgaacaagttgaaatcgttggacctatcatgatcatcaacggcattgaatgttcatttgatgaggaggaggatccttatttatatatgctccctcatcacacacatattttcgatttgcaacatatagttttttcagattatctagacagatatgtttcagaaaatgaatggaaccatgtggagatcacatactcagtggagcagagattcaataaaaaagacaaacatgcggtgaccccaatctctatagaaaatggaatctatgtgttgaaacagaggagcagcatggaggatattcaattcactgatccccacaaaaagagaagattagatgttgtctaaccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg

SEQ ID NO: 28 - DNA, 8577 bp, synthetic construct
  misc_feature 1..8577: note = Transient expression vector with Phaseolus lunatus FIT1
  source 1..8577: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatgtctgtgtcatcattctcctcttccttcacctatgacgtcttcctcagcttcagaggagaagacactcgttacggtttcaccggaaatctctattcagcccttcgtgacggcggaatttgcaccttcatcgatgacgaggagctccccaaaggagacgaaatcaccactgcacttgagaaggctatcgaaggttccagaattttcatcatcgttttctctcacaactacgcatcttcttccttttgcttgaatgagctcgcctacattcttccctacgctaagagaaatggtttgctggttttgccactcttctacgacgtcgttccttcccacgtgcgaaaccacacgggtacctttggagaagcgttagatactcatgaaaacaggttcaaagctacgagtgagggttttgagcttaacatggagaagctcaacagatggaagatggctctgcgtggagctgctaacttatctggctatcatttcaaacatggggaggaatatgaataccagtttatcaagaggatagtggaattggtatctaacaagattaaccgtgctcctttacatgttgcggattatccagttggactagagacacgagtgctagaagtaaaattgcttctagatgtaggatctgatgatggtgtccacatggtagggatccatggattcggtggagttggtaaaaccacacttgctcttgcagtttacaattccatagctgacaattttgaaggcttgtgtttccttgaaaatgtcagagagaattcaaacaaacacggcctacaacatcttcagagtctccttctttctcaaatggttggagaaaataatatcaacataactagtgtgaaacaaggaatttcaatgatgcagcatagactacggcagaagaagattcttttgattcttgatgatgttgacaagcatgaacagttacaagcaattgttggaagagctgattggtttggtcccggcagtagagtgatcatcacaactagggacaaacatttgttgtcatgtcagttggttgaaaaaatatatgaagtgaagaagttgaaaaagaaagatgctcttcgactgcttagttggaaaggttttaaaacagaagaagttcatccaagttatgtgaatgtcatggatcgtgtactggcttatgcttctggccatccattggctttggaagtaataggttcgaagttgtttagaaaaagtgtaaaagaatgggaatctgccatccaccagtatgagaaaattcctaacaatcaaattctggagatgcttaaaataagttttgattctttagaggaagtagagaagagtgtttttcttgacattgcttgttgtttcaaaggatatgcgttgtcagaggtggaaaatatacttcgtgctcattatggtgattgcatgaaatatcatattggggtgttggttgaaaaatctctcataaaatatggatggaattctgtagttacattgcatgacttgatagaagacatgggtaaagaaattgtccgacagaaatcaccaaataagccagggaagcgcagtagattatggtcaccagaagatataattcaagttttggaagacaactcgggaagtggagaaattgaaatcatatgtttgaattactctttacctgacaaagcagaaatagtagaatggaacagaaaggccttcaagaagatgaaaaacctcaaaacacttatcattaaaagtggtaaattttcggaaggtcctaagtatcttccaaacagtttaagagtaatggaatggttaaaatatccttcacaagggctaccgccagattttcgttcaaaggaacttgccatatgcaaattacctgcaagttgttttgggtcactcgaattggccgagttatcaaagaagttcaagaatatgacccttttgaattttgacgaatgtgaaggtttaacacagatacctgatgtatctgggctgccaaatttagtagaaatttcattcaagaattgtaagagtttaattacaatccatgactccattgggttcctaggtaagcttatctccttgaatgctgttggttgcagcaagcttaggagtttcccccccctcaaattgacttctctggaaaatcttgaactttcatattgttacagtcttgagagcttcccagaaatattgggaaaaatgggaaagataacagaacttttgttggagtgctgtgacataaaagaattgccattttcatttcaaaatctcactgagcttcaaacattacagttgcgttactgtccaatgttgaggttgccaagtagtattgtcatgatgccaaaactgaccgagattattgcttgggaatctaaaggatggctatttccaaaacaggttgagggtgaagagaaagtaagctcaatggtgtcttcaaatgtagattgtcttcttctcccagggtgcaaactctcggatgattttttcccaataacatggtttgctaatgtaaaagagttagacctgtcaaggaatactttcacggttcttcctgaatgcatcagcaattgtcattttttatgtaagcttattttagataactgtcagagtcttcgagagattagagggattccgccaaacgtacgacatttatcagcaagatattgtaaatccttcacttcttcttgcagaagcactttactgaatcagaaactgcatgaggcgggaaacaccatgttttggttttcaggagcaagttttccagaatggttcgagcaccacagtcggggaccatcttgttctttctgggttgacaagaaattccctgccattgctctttgtattgttattggaccaactcatttggaacatactgagattgttggacctatcataatcatcaatggcgttgattgttcacttgttgatgagttggataatccttatttacggatactccctcatcacacatatcttttcgatttgcaacatatagtttcttcagattatctagacagatttgtttcagaaaaggaatggaatcacgtggagattagatactcagtcgagcagagattcagtgaagaagacaaacatgtggtgataccagtctccgtagaaaatggaatctatgtgttcaaacagaggagcagcatggaggatattcaattcactgatccccacaaaaagagaagattagatgttgatccagagtagccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg

SEQ ID NO: 29 - DNA, 8583 bp, synthetic construct
  misc_feature 1..8583: note = Transient expression vector with Vigna radiata FIT1
  source 1..8583: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatggctgtggcatcattctcttcttccttcacctatgacgtgttcctcagcttcagaggagaagatactcgttacagtttcaccggcaatctctacagagcccttcgtgacagaggaattcacaccttcatcgacgacgagaagcttcccaaaggagacgaaatcacctctgcacttgagaaggcaatagaaggttccagaattttcatcatcgttttctctcgcaactacgcctcttcctccttttgcttgaacgagctcgcctacattcttccctacgctaatagaaatggtttgctggttttgccactcttctacgacgtcgttccttcccacgtgcgccaccacatgggtagctatggagaagctttggatactcatgaaaacaggttcaaagctacgagtcagggttttgagcttaacatggagaagctcaacaaatggaagatggctctgcgtggaacagctaacttatctggctatcatttcaaacatggggaggaatatgaatacgagtttatcaagaggatagttgacttggtttccaacaagattaaccgtgctcctttacacgttgcggattatccagttggactagagactcgagtgctagaagtaaagttgcttctggatataggatctgatgatggtgtccacatggtagggatccatggactcggtggagttggtaaaaccacacttgctcttgcagtttacaattccatcgctgaccattttgaaggcttgtgtttcctcgaaaatgtgagagagaattcaaacaaacacggcctacagcatcttcaaagaatccttctttctcaaatggttggagaaaataatgtcaacataactagtgtgagacaaggaatttcaatgatgcagcataggctacgacagaagaagattctcttgattttagatgacgttgacaaacatgaacagttacaagctattgttggaagacctgattggtttggtcccggcagtagagtcatcatcacaactagggacaaacatttgttgtcatgtcacttgatcgaaaaattatataaagtgaagaagttgaaaaagaacaatgctcatcgactgcttagttggaaagcttttagaacagaagaagttgatacaagttatttgaatgtaatggatcgtgtactagcttatgcttctggccatccattggctttggaagtaatcggttcgaagttgtttagaaaaagtgtaaaggaatgggaatctgccatcaaacagtatgagaaaattcctaacaatcaaatcctggaggtgcttaaaataagttttgatgctttagaggaagtagagaagagtgtttttcttgacatttcttgttgctttaaaggatatgcattgtcagaggtggaagatatacttcgtgctcattatggtgattgcatgaaatatcatattggggtgttggttgaaaagtctctcataaaatatggttggagttgtgtggttacaatgcatgacttgatagaagacatgggtaaagaaattgtccggcagaaatctccaaataggccagggaagcgcagtagattatggtcaccagaagagataattaaagttttggaagacaacttggggagtggagaaattgaaatcatatgtttaaattcctccttacctgacaaagaagaaatagtagaatggaacagaaaggccttcaagaagatgaaaaacctcaaaacacttatcattaaaaatggtaatttttcggaaggtcctgaatatcttccaaatagtttaagagtactggaatggttgaaatatccttcacatgggctaccgccagattttcgttcaaagaaactttccttatgcaaattaccttcaagttgttttgggtcactcgaattggctgagttttcaaagaagttcatgaatatgactcttctgaattttgacgaatgtgaaggtttaacacagatacctgatctatctgggatgccaaatttagaaagattttcattcaagaattgtaagagtttaattacaattcatgactccattggcttcctaggtaagcttaactccttgaatgctgttggttgcagcaagcttaggagttttccccccctcaaattgacttctctggaaaatcttgaactttcatattgttacagtcttgagagcttcccagaaatattaggaaaaatgggacagataacagaacttgtcttggaggactgtcacataaaagaattgccaatttcatttcaaaatctcaccgagcttcgaacattacagttgcgttcgtgtccaacgttaaggttacctagtagtattgtcatgatgccaaaactggccaacattattgcttgggaatctaaagggtggctatttccaaaacaggtcgagagggaagagaaaataggctcaatggtgtcttcaaacgtagattgtcttgttctctcagggtgcaaactctcggatgactttttcccaataattcttgaatggtttgctaatgtaaaagatttaaacctatcaaggaataacttcacggttcttcctgagtgcatcgccaattgtcacttgttatgcaagcttactttagatgcctgtcatagtcttcgagagattagagggattccaccaaacatacgacagttactagcaagaaattgtaaatccttcacttcttcttgcagaagaactttactgaatcagaaactgcatgaggctggaaacaccatgttttcgttttcaggagcaaggtttccagaatggttcgatcaccacagtcggggagcatcttgttctttctgggttggcaaaaaattcccttccattgctctttgtattgctattggaccaactcatttggaacaccttgaaattgttggacctatcatgatcatcaactgcattgaatgttcatttgattgggaggagaatccttatttatatatgctccctcatcacacatatattttcgatttgcaaaatatagtttttccagattatctagacagatttgtttcagaaaatgaatggaaccacgtggagattacatactcagtcaagcagagattcaagggaaaagacaaacatgaggtgaccccaatctctatagaaaatggaatctatgtgttcaaacagaagagcagcatggaggatattcaattcactgatccccacaaaaagagaagattagatgttgatccagagttgtaaccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg

SEQ ID NO: 30 - DNA, 8580 bp, synthetic construct
  misc_feature 1..8580: note = Transient expression vector with Vigna unguiculata FIT1b
  source 1..8580: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatggcagtgccaagcttttccttcagttttacttatgatgtctttttaagctttagaggagaggacactcgttacggttttacgggaaacctttaccgagccctccgtgacagaggtatacataccttcatcgatgacgaggaattaagaaaaggagacgaaataaccagtgcattagagaaagccatcgagggatcacgtattttcatcatagtatttagtcttaattacgccagtagctctttctgtttgaatgagcttgcatatattcttccgcacgctaaaaggaacgctcttctggtgctcccccttttttacgatgtggttccaagtcacgttcgtcaccacaccggatcattcggtgaggctttagatgcccatgagaaacgttttcgaggcatgagtcagggttttgaattaaacatcgagaaactcaataaatggaaaatggccttacgaagggcagcaaaccttagcggatatcatttcaagcatggcgaagagtatgaataccaatttatagaaaaaattgttaaactcgtttccaataagataaacagagttccgctgcacgttgctgactatcaagtaggactcgaaaagagagtcttagaagtgaagatgttagttgatgtgggatcagatgactgcgtgcacatggtgggtatctatggtttaggaggagtggggaaaactacactcgcactggctctgtataatagtattgcagaccactttgaggggctctgctttttggagaacgtcagagagaattcaaacaaacagggcctccatcatctgcagcgtattctcctgtcagaaatggtgggagagaacaacataaatatagggagtgtgcgtcagggtatttcaatgatgcaacatcgtttaagacagaaaaaaattcttttaattttggatgatgtagataaacacgagcagcttcaagctatagttgggagtcctgattggttcgggccaggttcccgagttatcattactactcgtgataagcagcttttgtcttgccacttagtggaaaagctttacgaggtcaaaaaacttgaaaaaaacgacgctcttcgactcctgtcttggaaaggatttaggactgaggaagttgacacatcttattctaacgtcatggactccgttttagcatacgcttccgggcaccccttagctttggaagttatagggagcaaactgttccgaaaaagtgtagaggagtgggaatccgctatcaaacaatacgagaaaataccgatctcccagatccttgaagtattgaaaatttcctttgacgccttggaagaggtggagaaaagcgtgtttctcgacatagcctgttgtttcaagggttatgccttgtctgatattgaggatatattacgtgcacagtacggacacagtatgaaatatcacattggtgttttagtagagaagagtctgattaagtacaaatggaaattcggtgttaccatgcacgacttgatagaggatatgggaaaggagatcgtccgacaacagagcccaaaaaggcccggaaaacgaagtaggctctggtcccccgaagatataattcaggtattggaggataacagcggttctggagaaatcgagattatttgtctgaatagtagcctgcctgataaagaggaaattatagagtggaacagaaaagctttcaagaaaatgaaaaatttaaaaacattgataataaaaaaagggaatttcagcgaggggccaaaatacttgcctaatagtctgagggttttggaatggttaaagtaccccagccaaggattgccacccgactttcgttccaaagaattggctatttgtaaactgcctacctcatgcttcggcagtcttgaactggccgaacttagtaagaaattcatgaacatgactcttctggattttgaagagtgccaagggcttacgcagataccggatgtgtctggcatgccgaatttagaaaagatcagttttaaaaattgcaaaagtttgaacactatacatgatagtataggcttcttggggaaattaaatagtctgaacgcagtagggtgctccaaactgcgtagcttccccccgctgaagttaacaagtctgaaaaatcttgagcttagctattgttatggtttagaatctttccctgaggtactgggcaaaatgggaaaaatcacaaaacttgtcctggaggattgcaacatcaaagaattaccgttctcttttcaaaacttaacagagctccaaacgcttcagttacgtttctgccccatgctcaggttaccgagtagtattgtaatgatgcccaaattagctgagattatcgtgtgggagtcaaagggatggctgttcccgaagcaggtagaagtcgaagagaaagtctcatcaatggtatcatccaatgtagattgtttacttttacccagatgtaagctctctgacgactttctgccgatcgcccttgcctggtttgccaacgtgaaggagttggatctcagttggaacaacttcaccgttctcccggaatgcatatcaaattgccatttcctgtcaaagttgacattagatggttgccacagcctcagggagatacgaggaattccaccgaatatccaactcctgcttgccgttgactgcaagtccttcacaagcagctgtcgttctacattgttaaaccagaagctgcatgaagccgggaacacaatgttccgtttgagcggagcatcatttcctgaatggtttgatcaccacaaccagggcccgtcttgtagtttctgggtcggaaacaaattcccaagcatagccctttgtatagcaattgggcctgcccacctggaacacgtcactattgaccgtccgattataaatatcaacggcgtcaagtgtagtctccacggggaagaaaagccttatcttaatatgctcccgcatcatacctacctgtttgatctgcagcacatcgtctttagtgactatcttgaccgattcgtcacggagaatgagtggaatcacgtcgagattacatactctgttaagcaaagattcaatgaaaaagacaaacatgcagtgacgcccgttagcgtcgagaacggtatttatgtgctcaaacagagatcatcaatggaagacattcagtttactgaccctcacaaaaagagacgactcgatgtagatccagagtaaccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg

SEQ ID NO: 31 - DNA, 8583 bp, synthetic construct
  misc_feature 1..8583: note = Transient expression vector with Vigna angularis FIT1
  source 1..8583: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatggcagttccaagtttttcatctagcttcacatatgacgtcttcttgtccttccgtggggaggacacaaggtattcattcaccggcaacttgtacagagcactgagggatcgaggaatccatacttttatcgacgatgaaaagctgccgaagggcgacgagatcacatctgcccttgaaaaggcaatcgagggcagtagaattttcattattgtattttctcgaaactacgcctccagctcattctgcctgaacgagttggcatacatcttaccttacgctaataggaacggtcttttggtacttccactgttttatgatgtggtaccgagccatgttagacaccatacgggctcattcggagaggctttagacactcacgaaaacaggttcaaggccacatctcaaggattcgaattaaatatggagaagttaaataagtggaagatggcacttcgaggtactgctaacctgtcaggatatcatttcaagcacggcgaagagtatgagtatgagtttataaaaaggatagttgacttggtttctaacaaaataaacagggccccactccatgtggcagattatcctgtaggacttgagacgagggtactcgaggtcaaattgttattagatatagggtctgatgatggcgttcatatggttgggatccatggactgggaggtgtgggcaaaacaactcttgcactcgcagtttacaatagcattgctgaccattttgaaggcctgtgttttctggaaaacgtaagagagaattcaaacaagcacgggctgcagcacctgcagcgtatcctcttatcacaaatgataggcgagaacaatgtgaacattacatctgccaggcaaggtatcagcatgatggagcatagattaagacagaagaaaatcctgttgatcctggacgacgtagataagcatgagcaattacaagcaatcgtaggacgtcccgactggttcgggccgggatccagagtgattataacaacacgagacaaacaccttcttagttgtcacctgatcgaaaagttgtacaaggttaagaaactggagaaaaacaacgcacttaggttgctctcctggaaagctttccgtacagaggaggtagatacttcataccttaacgtgatggatcgagtcttggcctatgcatccggacatcctttggctctcgaagtcattgggagcaagctcttccgtaaaagtgtcaaggagtgggagtccgccattaaacaatatgagaagattcccaacaatcaaattctcgaagtcttaaaaatatcctttgacgctctggaagaagttgagaagagcgtctttctcgatatttcttgttgtttcaaagcctacgcactcagtgaagtggaggatatactcagagcccattatggggactgcatgaagtaccacataggcgtactcgtcgagaaatctttgattaaatacgggtacaattccgtggtaacgatgcatgaccttatcgaggacatgggtaaggagatcgtccgtcaaaaaagcccgaacaaaccaggaaagcgttccagactctggtcacccgaagagattattaaagttttggaggataatcttggctcaggcgagatcgaaattatatgtctcaacagtagcttacccgataaagaagagatagtggagtggaatagaaaggtcttcaaaaagatgaaaaacctgaaaactctcataattaaaaatggaaatttctcagaaggacccgaatatttgccaaactcactccgtgtcttagagtggctcaagtaccccagtcaagggcttccccccgattttagatccaagcaactgtccctctgcaagctgccctcctcttgcttcggatctttgggacttacagagttttctaagaagtttatgaatatgactttactcaattttgacgagtgcgaggggctgacccaaatacccgatttaagtggattaccgaatttggaacgatttagttttaaaaattgtaaatccctgataactattcacgactctatcggcttcttaggtaaactcaattcacttaacgccgtcgggtgtagcaagcttcgttcttttcccccgttgaaattgacttcacttgagaatttagagttgagctattgctactcattggaatctttccccgaaatcctggggaaaatggggcagatcaccgagctggtcttggaggactgtcatatcaaagaattgcctatttccttccaaaatctgacagagttgcagacactgcaattgagatcctgtcccagattgagactcccttcctcaattgtgatgatgcccaagttagctaatatcattgcctgggaaagcaagggttggttgtttccaaaacaggtggagggtgaagagaagataggctccatggttagttccaacgtggactgtctgtacctgtccggttgtaaattgtctgatcatttcttcccaataatcctcgaatggtttgccaacgtaaaggatttgaatcttagcaggaacaactttacggtgctccccgagtgtatcgctaactgccatctgctttgtaaattaacgcttgatgcctgtcactcactgcgtgagatccgtgggatcccgcccaatattcgacaacttagcgcaagaaactgtaagagcttcacatcttcatgcggccgaacattgctgaatcaaaagctgcatgaagcaggtaatacaatgttttccttctctggcgcaagatttcctgagtggtttgatcaccatagttgggggccttcatgctcattttgggtgggtaagaaatttccaagcatagccctgtgtatagctattggtccaacgcaccttgagcatgtagagatagtagggccaattatgattatcaattcaatagagtgttcattggatgaggaggaaaacccctacttgtatatgctcccgcatcacactcatatattcgaccttcagcatattgtattttccgattatttagataggttcgtatccgagaatgaatggaatcacgtggagattacttattccgtcgagcaacgatttgaagagaaggataaacacgctgtaactcaaatctccatcgagaatgggatatacgtctttaagcaaaactcctctatggaggatatacaattcacggaccctcataaaaaaaggagactcgacgtcgatccggaactgtaaccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg

SEQ ID NO: 32 - DNA, 8577 bp, synthetic construct
  misc_feature 1..8577: note = Transient expression vector with Phaseolus vulgaris FIT1
  source 1..8577: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatggctgtgccgagcttctcatccagcttcacgtatgatgttttccttagtttcagaggggaggacacgcgttatggattcaccgggaatttatatacggctttgcacgattccggcatctgcacttttatagacgacgaggagttgccaaagggcgacgagataacaacttccctccagaaggcaatagaggcaagtaggattttcataatagtatttagtcacaattatgccagcagttcattctgtttaaacgagctcgcttatatactcccctatgccaagaggaatggcttattggtgctgcctttattctatgatgttgtaccctcacatgtaagacatcatactggaactttcggggaagcactggatactcacgagaataagttcaaaggaacctctgaaggcttcgaattaaacatggaaaaactcaatcgttggaaaatggctcttcgtggggcagctaatttgagtggataccactttaaacacggcgaggaatatgagtaccaatttatcaaaaggattgtcaagctcgtgagcaacaagattaacagagcaacacttcatgttgcagactaccctgtgggcttggaaacccgtatgctcgaagtaaaacttcttctggacgtaggttccgacgacggcgtacacatggtgggaatacatgggttcgggggcgtcgggaagaccacacttgctctggctgtgtacaattctattgctgataattttgaaggcctgtgctttttagaaaacgtcagggagaactccaacaaacatggattgcagcatttacagtccctgctgttgtctcaaatggtgggtgaaaacaacatcaatataaccagtgtcaaacagggcatatcaatgatgcaacatcgactccgacagaaaaaaattctgctgattctggatgacgtagataagcatgagcaacttcaggcaatagtaggacgagccgattggttcggacccggatctcgagtaattataactacgagggacaaacatttattgagctgccaactcgtagagaaaatttacgaagtaaagaagcttaaaaaaaaagatgccttgaggctcctttcatggaaggggtttaaaactgaggaagtgcatccgtcttatgtgaacgtaatggggagggtcctggcctacgccagcggccacccactcgctcttgaagtcattggatcaaaattatttcgtaagtccgtcaaggaatgggagtccgcaatacaacaatatgaaaaaattccgaacaatcaaatcttagaaattcttaaaatttctttcgattctttagaggaggtggaaaagagtgtatttcttgacattgcctgctgctttaagggatatgcactgagtgaagttgagaatatcttgagggcacactacggggactgcatgaagtatcatataggagtactggttgagaagagtttgatcaaataccgttggaactctgttgtgaccttgcacgaccttatcgaggacatgggtaaggaaatagtcaggcaaaaatctcctaataagcccggcaaaagatcccgtttatggtcacctgaagacatcatccaggtcctggaagataatagtggatcaggtgaaatagaaatcatctgcctgaattactcacttcccgggaaagaagaaattgtcgagtggaacaggaaggctttcaagaaaatgaaaaatctcaagacactcataattaaatctggaaatttttcagaaggtcccaaataccttccgaattcactccgtgtgatggagtggctgaaatatccatctcaaggcctccccccagatttcaggagtaaggaattaacgatctgtaagttacccgcatcctgcttcggcagcctcgagttagctgagttatctaagaaatttatgaatatgacgttattaaatttcgacgaatgtgagggcctcacacaaatccctgacgtctctggactgccgaacttagtagagatctctttcaagaactgcaagagcctgatcactattcacgattctatcggattcttagggaaactcaatagcttgaatgctgtaggctgctctaagctcaggagcttcccacctttgaaattgacatctcttgaaaaccttgagttgagttattgttatagcctggagagcttcccggaaatcttaggaaagatgggtaagattaccgagctgtttttagagtgctgcgacataaaagagcttcccttctcttttcagaatctcacagaattacagactctgcagctccgatactgcccgatgctgcgattaccctctagtatcgtgatgatgccgaaattgacagagattattgcttgggaaagtgagggatggttgtttcccaaacaggtcgaaggagaagaaaaggtgtctagtatggtgagttcaaacgtagactgcctcttactgcccggctgcaagctcagcgacgatttttttccgattacctggttcgctaacgtaaaggaacttgatctctctagaaacacttttaccgtcctgccagaatgtatttctaactgttattttttgtgtaaattgattttggacaactgtcactctctccgtgaaatacgtgggatcccgcctaacatacgacacttgtccgccaggtattgcaaatcattcactagctcctgtaggagcacgctcttgaaccagaagttgcacgagacaggcaatactatgttttggttttcaggcgctagttttcccgagtggtttgagcataggagtcaaggtcccagttgtagtttctgggtgggaaataaatttcctgccatcgcactctgtatagtgatcgggccaacacatcttgaccatatcgagattgtgggaccaatcatgatcattaacggcatcgattgtagcctcgatgatgggctggtcaatccctacctttggattccacctcaccatacgtacctgttcgacttacagcacatcgtttctagtgactatcttgatcgatttgtctctgagaaagaatggaaccatgtcgaaatcagatattcagtgaaacagcgtttttcagagaaggataaacatgtagtcatccctgtgagcattgaaaacggaatctacgtttttaagcaaaggagctccatggaagacatacagtttacggatccacacaagaaacgaagattagacgtagatccagaataaccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg

SEQ ID NO: 33 - DNA, 8580 bp, synthetic construct
  misc_feature 1..8580: note = Transient expression vector with Lablab purpureus FIT1
  source 1..8580: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatggcagagccctctttcagttcatctttcacgtatgacgtgtttctctcctttaggggcgaggacacgcgatactcttttacaggcaacctctacagagctttacgagattctgggatatgcacctttatcgacgatgaagagttgaggaagggcgacgagataaccagtgccctcgaaaaagctatagaaggttcacgaatattcatcatcgtcctcagtcataactacgcaagctcctccttttgtcttaatgaactggcatatattttgccatatgccaagaggaaagggctcctcgtgctccctctcttttacgacgttgtcccgagccatgtacgacatcatactggcagcttcggagaagcactggatactcatgagaagcgatttaaagggacatcagagggttttgaactcaatatggagaagttgaatgaatggaagatggctcttagacacgttgccaatttttcaggttaccatttcaaacacggtgaagagtacgaatatgagttcattaaaaaaatagtggagttggtgtccaacaaaatcaaccgtgctcctttacacgtagctgactaccctgtagggttggaatcccgagtactggaaatcaagctgctcttagatgtcggcagcgatgatggagtgcatatggtgggaatctatggcctcggcggggtggggaaaacgacgttggccgtagcagtctataatagtatagctgaccattttgaaggtttatgcttcctcgagaatgtaagggaaaactcaaacaaacacggccttcaccatctccaatcactcttactctctcaaatggtcggggagaaaaatatcaatattagttctgttaagcagggcatctccataatgcaacatcgacttaggcaaaagaagattctgctcatccttgatgatgttgataagttggaacaattgcagtcaattgtaggacgacctgactggtttggcccaggctctcgtgtgataataacaacgagggataaacagcttctcagctgtcatcttgtggaaaagacttatgaagtaaagaaattgaagaagaaggacgccttccgattgttaagctggaaaggttttagaactgaggaagtagacacttcctacgtaaacgtgatggaccgtgttttggcttacgccagtgggcatccgttggctctggaagttattggatccaagctttttagaaaatccatcaaggaatgggagtcagctatcaaccaatacgaaaaaatccccaataaccaaatacttgaaatactcaaaatatcatttgatagcctggaagaagtggagaaatccgtctttcttgatatatcctgttgcttcaaaggctatgcccttagtgaagtggaggacatattacgtgcccattacggggattgcatgaagtaccacatcggggtcttagtagagaagtcattgattaaataccgatggggttgcatcgtgactctccacgatttgatagaagacatggggaaggagatagtgcgacagaaatctccccagaagccaggaaagaggtcacgtctgtggagtccggaggatatcattcaagttcttgaggacaattctggcagtggtgagatcgagattatttgccttaattcctctttattagataaagaagaaacaattgagtggaacagaaaggctttcaagaagatgaaaaacttgaaaacgttaataataaagaacggaaacttctcagagggaccgaaatacttgcccaatagcctgcgagtgttggaatggctcaagtacccaagtcaaggtctgccccccgacttcagatctaaaaaacttgtgatttgtaaattgccttcctcttgtttcggcactctggaacttgcagagttatctaaaaagtttatgaacatgactgttctcaattttgatgaatgtgagggtcttacgcagatcccagacgtgagtggcttacccaacttggagaatatcagtttcaaaaactgtaagtcccttattacgattcacgactctatcgggtttcttgataagcttaattccctcaacgccgtgggttgctcacgactgagaagcttccccccactgaagctgacatcattggagaacctcgagttatcttactgctatagcctggaatgtttccctgagattctgggcaagatggaaaagatgacggaactggtcttggaggattgtgacataaaggagattccgttctcttttaaaaacctgactgaattgcagactctggaccttcgtttttgtaggatgcttcgattgcctacttctattgtcatgatgccaaaattgtccgaaattatcgcctgggaggctgagggatgcttgttccccaagcaagtcgagggcgaggaaaaggttagttctatggtcagttcaaatgtagactctctcagactcagcggatgtaagctctcagatgattttttcccaaccactctcgcttggttctccaatgtgaaggacttgaacctctctagaaataactttactgtattgccagaatgtatcagtaattgccacttcttgtataaactgacactcgactactgtcattccctccgtgaaataaggggcctccctcctaacattcagcacttgtccgcaattaattgtacctctttcacctcatcatgccgttccacattgctgaatcagaagctccacgaggctggtaacacaatgttcagctttagtggtgcaatgtttccggaatggttcgaaaatcactctcgaggcccgtcttactccttctgggtaggaaacaaattcccggcaatcgcattatgcatagctatagggcctactcatgtggagtatgtgcagatcgtaggcccaatcatgattataaacgggattgagtgctctttaccaggtgtagatccttatcttaagatgctgcccgaccacacatatttgttcgacctgcaacaaattgtgttcccagattaccttgacagatttgtttctgagaacgaatggaatcatgtcgaaataacatatgccgtggaaaaacgtttctcaaagaaagacaaacatgtcgttatacctgtctctattgaaaacggcatatatgtcagcaaacaaaggtcttccatggagaacgtaagattcacggatcctcgtaagaagagaagactcgacgtggacagcgaattgtaaccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg

SEQ ID NO: 34 - DNA, 8559 bp, synthetic construct
  misc_feature 1..8559: note = Transient expression vector with Abrus precatorius FIT1
  source 1..8559: mol_type = other DNA
atctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgctagccaattggggcccaacgttctcgaggggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggatctagaaggccttggatccacccgggagaattcgtcgactttgcggccgcatcgatactgcaggagctcggtaccgagcatggcagtgcgtagttgctcttcttctttcacatacgcctttacttacgatgtttttctgagctttaggggatccgatacacgtcacggattcgtcgggaatctgtttaaagccttgcaggataagggaatccatacgttcattgatgatgagggactccaacgaggtgaagagataagtcctgcattggtaaaggccatagaggaaagccgaatcgccatcgcagtccttagcaataactatgcttatagtagtttctgcttagatgagcttgcccacatattagagtgtgtcaagcgtaaagatacgttggtcttacctcttttctatgatgttgacccgagccatgtgcgatatcaacgaggctcttacggcgaagcacttgcttcccacggcgaacgttttaagcataacatggaaaaactgcagaagtggaaaatggccctgcatcatgttgctaatctctcagggtatcactttaaacatggggatggctacgagtatgagttcataggacgaattgtcgagcttgtatctaacaagatcaatcgtgctccattgcatgtagccgattatccagtgggtttagagtcacaggtacttgaggttaggaaacttttggacgtcggctcagatgacggggtgttgatgattggtattcatggaatcggtggtatcggtaagactactcttgccttggctgtctacaatctgattgctgatcatttcgatggattatgtttccttgagaacgtcagagaaaactccgacaaacatgggcttcagcacttacagagtatactcctttccgagattcttggtgaaacgaaaatcaaactcgcaagtgtgaagcagggaatctccatactgcagcaccgtttgcaaaggcagaaggtgctgctcatcttagacgacgtggacaagcatgagcaattaaatgcaatcgtgggaaaaccggattggttcgggcctgggtcaagggtaattatcacaacaagagacaaacaccttctcgcttgtcacgaagtgaagagcactcatgaagtcaaaaaactgaataagaacgacgccttgcagctcctttcctggaaagctttcaaatcagagaaagtggacccaagctatgtcgacgtcatgaactgcgctgttgcatacgcatcagggcaccctctggccttggaggtaattggaagtaatttattcggtaagagcattgaacagtggaaatctgctattaatcagtacaaaaggatccctaactgtcacatcttaaaaatcttaaaagtatcatttgatgcactggaagaggaggaaaagagtgtattcttggacattgcatgctgtttcaagggctacaatttggaagaagtcgaggacatcctgcacgcacattacggggactcaatgaagtaccatattggcctgttagtggagaagtcactgataaagctgacctgggccggtggcgttacgcttcacgacttaatagaagacatgggtaaagaaatagtgagacagaagtccccaaaagaccctggaaagcgtagccgattatggctgcctgaggacatcattcacgtcttcgaacatgacagtggcactggcgagattgagataatacgtcttgcctctagtctgttagataaggaggagattataaagtggaatcgaaaggcttttaagaaaatgaagaaccttaaaacactgataatcaagaacgggcatttttccaaaggtccaaagcatttgccaaatagcttaagggttttggagtgggcaaaataccccagtcagggatttccagctaatttttgttccaagaagctgagcatctgtaagctcccgaaaagttgtttttccttagaacttgcagatctcagtgagaaattcatgaatatgtccgtacttaatttcgatgagtgcgagggattgacgcaaatcccggacgtgtcaggattacagaaccttgaaaaattttcatttaaaaattgtgaaaatcttatcaccatccatgacagcatcggtttcctgcacaaactcaagttcctgaatgcaatcggctgccgtaaacttaggagcttcccacctttgaagctgacctcacttgaaaagcttgagttgtcttattgctcaaatcttgaatgttttcccgagatactgggcaaaatggagaacataaccgaacttgttctcgaggctagcgcaattaaggagttgcccttttcctttcagaacctgacaggcctccagatcctccaactgagactgggtggaatgattagattgccgtcatcaatcgttatgatgcctaagctgacagaaataatagcatgggattggaagggtttgctttggccgcgtcaagtcgagggggaagagaaggtgtcctccatggtgtctagcaatgtcgactgcttatgtctgtctggatgtaatctttctgaccagttcctccccgtggcccttagttggtttgttaatgtgaaagatttggacctctctcgaaataaattcacagttctcccggagtgtatttccgagtgccatttcctctggaagcttatcctcgactattgtaactgtcttagagagattcgtggaatgccaccaaatattgaacatttcagcgctagaaattgcaagtcacttactagttgtaggagtacccttctgaaccaaaagctccacgaggctggaaacacaatgttctggttgagtggaacgtggtttcccgaatggttcgagcatcacagcaagggcttgagcaattctttctggttccgagacaagtttccggccatagctttgtgcacagccatagggcccactagggaacagataaccatagtcggcccgatagtcattattaatggaattgaatgcagtgttgatgatgaggacgatagttatttgtggatggaaaccgaccacacttacctttttgatttgcaaaagatcaattttgcagacaatttagataaggaattagtcgagaatgagtggaaccatgtggaaataacgtatagcgtaatttctaacgagaaggagaagcacgttgagatcccagtcttcattgagtcaggcatttatatatttaaacaaagatcccgaatggaggatatccgatttaccgacccttacaagagaaggaagctcgacgatggtttagagagttaaccaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgg